(19) United States
(12) Patent Application Publication
(10) Pub. No.: US 2025/0314647 A1
(43) Pub. Date: Oct. 9, 2025

(54) SACCHARIDE COUPLED MICROSPHERE, METHODS OF PREPARATION AND APPLICATIONS THEREOF

(71) Applicant: SERUM INSTITUTE OF INDIA PRIVATE LIMITED, Pune (IN)

(72) Inventors: Laxmikant Vyankatrao KADAM, Pune (IN); Manish Mahesh GAUTAM, Pune (IN); Sunil Jagdish Prasad GAIROLA, Pune (IN); Amol Subhash MURKUTE, Pune (IN); Aniket Sunil BHUJBAL, Pune (IN)

(73) Assignee: SERUM INSTITUTE OF INDIA PRIVATE LIMITED, Pune (IN)

(21) Appl. No.: 19/169,195

(22) Filed: Apr. 3, 2025

(30) Foreign Application Priority Data
Apr. 4, 2024 (IN) 202421027992

(51) Int. Cl. G01N 33/543; G01N 33/68

(52) U.S. Cl. CPC G01N 33/54313; G01N 33/6854; G01N 2333/22; G01N 2333/285; G01N 2333/315; G01N 2400/10

(57) ABSTRACT

The present invention relates to an improved non-covalent (electrostatic) coupling of polysaccharides to microspheres to form couples where structure of saccharides remains unaffected/intact and retains epitope confirmation. The stable saccharide coupled microsphere obtained by the method is used for determining the immunogenicity of the immunogenic composition including antigen content determination, identity assay, free saccharide estimation and estimating antibody concentration (IgG) in sera sample. The method is rapid, simple, repeatable, cost effective, scalable, non-toxic to couple microsphere to saccharides. The method results in efficient coupling by selecting correct saccharide size, concentration and pH

### TECHNICAL FIELD

The present invention relates to the field of saccharide-protein conjugate vaccine(s) and particularly to saccharide coupled microspheres used to evaluate the saccharide-protein conjugates in vaccines/immunogenic compositions.

### BACKGROUND OF THE DISCLOSURE

The background information herein below relates to the present disclosure but is not necessarily prior art.

Saccharides (hereinafter interchangeably referred to as PSs, saccharides, polysaccharides, capsular saccharides, oligosaccharides) are carbohydrates and are abundantly found in variety of organisms. Saccharides are seen on capsule, cell walls and other cell surfaces of the organisms, microorganisms, bacteria, yeast, fungi, and viruses. Capsular Saccharides have epitope motifs usually not found in mammals and are used to mediate immunogenicity. Such saccharides are therefore useful for the preparation of vaccines against bacterial diseases such as meningitis, pneumonia, and typhoid fever.

Combination and polyvalent vaccines not only provide protection against several different pathogens at the same time but can also increase vaccine protection against pathogens that have closely related pathogenic strains or serotypes. In particular, the use of polyvalent conjugate vaccines for Streptococcus pneumoniae is important due to the organism's many serotypes, each with a distinct polysaccharide capsule.

However, testing the vaccine immune response to each serotype can be extremely time-consuming and laborious if each component must be assessed in an individual serological immunoassay. Therefore, multiplexed serological testing methods have been developed for determining the efficacy of combination and polyvalent vaccines.

Multiplexed immunoassays reduce the number of assays needed to confirm immune responses and cross-reactivity, use less serum, and can be performed faster. Testing in multiplex reduces the variables that must be controlled when performing individual tests and can thus be more reliable and ultimately are more cost-effective than single-plex methods.

Immunoassays as well, rely on detection of antibodies corresponding to saccharide antigens to diagnose infectious diseases and to assess safety and efficacy of saccharide-based vaccines. Diagnostic assays/immunoassays that detect antibodies corresponding to saccharide antigens (such as antigen content determination assays, identity assays, potency assays, immunofluorescent assays, Western blotting, enzyme-linked immunosorbent assays (ELISAs), radioimmunoassay (RIAs), and the like), use solid phase matrix to which saccharides are bound/immobilised or employ a liquid phase with suspended polysaccharides. For better results and detection purposes, the saccharides are coupled with microspheres.

Bead-based suspension array technologies are often used for development of multiplexed serological assays to simultaneously assess immune responses to multiple antigens and have been used in vaccine trials, testing in clinical laboratories, epidemiological studies, and in basic immunological research.

In particular, the xMAP® microsphere technology from Luminex has been used extensively for multiplexed serological assays to detect immune responses to a variety of antigens, including pathogens, autoimmune markers, as well as human leukocyte antigen (HLA) and alloantigens, which is important for donor and recipient testing in transplantation (Das and Dunbar, 2020).

The immobilization of biomolecules or any other such entities can be achieved by coupling by (a) ionic interactions; (b) adsorption; (c) complexation (such as “metal-coordination” mediated coupling); and (d) covalent bond formation between active/stable reactive groups on the surface and specific functional groups on the entity to be immobilized. For example, particles (such as micro- and nano-spheres; nanotubes; metal particles including one or more metals with any size, shape, or composition; semiconductor particles; molecularly imprinted polymers (MIPS); magnetic particles; other dyed materials and the like) and microtiter plates are common solid matrices in many immobilization systems.

Preparing and maintaining the active, functionalized surface of the solids are important to assure immobilization of biological material for development of a sufficiently sensitive assay. Current procedures for immobilization of biomolecules on solid surfaces generally involve reactions of activated carboxyl, amino-, hydroxyl- or thiol-groups on the solid surfaces with the biomolecules. After activation of, or introduction of a functionalized spacer to, these groups, the activated groups provide sites on the solid surface for direct attachment of the biomolecules.

While immobilisation (on solid supports) or for formation of the saccharide coupled microspheres, the saccharides are bound by either non-covalent bonds or by covalent bonds. The selection of immobilization by either non-covalent bond or covalent bond depends on several factors like nature and function of biomolecule, compatibility as well as operating and storage conditions. Non-covalent chemical bonding/chemistry includes attachment by van der Waals forces, hydrophobic interdigitation, physical adsorption, ionic bonding, affinity binding and the like. Covalent binding includes binding through sharing of valence electrons between an atom on the solid surface and an atom on the saccharides by forming strong chemical bonds.

Non-covalent immobilization of saccharides onto solid surfaces (coating) is generally time, reagent, and labour consuming because the optimal coating conditions vary among Saccharides from different bacteria strains as well as between serotypes of the same bacteria. It also results in low loading capacity, high leaching and is sensitive to environmental changes.

Variability in conditions for non-covalent methods impacts accuracy and reproducibility of quantitative determinations as well as makes immobilization difficult for two or more different saccharides on same surface. Saccharides also tend to aggregate and render stability of the bonded surface or couples unpredictable and for a shorter shelf life. Aggregation is overcome by adding detergents, however such addition cause variation in further assays and determinations.

Since non-covalent methods of coupling generally relate to physical adsorption of saccharides to bead surfaces, the bonding is fragile in nature. Additionally, saccharides with neutral charge are not able to couple with beads using physical adsorption methods.

Covalent couplings overcome some of the problems associated with classical non-covalent coupling methods. However, covalent chemistries include oxidization and other chemical modifications of saccharides for coupling polysaccharides.

Modification in saccharide structure possibly shields the epitopes and can lead to loss of sensitivity or challenges in assessing the true antigenicity/immunogenicity. Modifications in antigen structures via covalent reactions can cause limitations and challenges in functions of analytics. Covalent modification of saccharide also introduces new structures which can lead to additional challenges of cross reactivity. Modification of the structure of the saccharide leads to challenges in multiplex assays having higher valencies.

Currently used functional groups for providing direct attachment sites, have a number of disadvantages. For example, most of these functional groups (such as N-hydroxysuccinimide (NHS) esters, isothiocyanates, and the like) are prone to hydrolysis in an aqueous environment and become non-reactive (i.e., chemically inactive) in a matter of less than an hour. Therefore, the use of such functional groups for attaching the biomolecules to the surface of solids may undesirably exhibit issues such as time-dependent variations in the quantity, repeatability, and uniformity of the attachment process.

Fray et al., Bioconjugate Chem., 1999, 10, 562-571 have reported a strategy in which particles are pre-activated with hydrolysis-resistant aldehyde functional groups, but low reaction yields of less than 8% have been observed with these microspheres. U.S. Pat. No. 6,146,833 to Milton describes a reaction between an acyl fluoride activated polymer-surface and an amino derivatized biomolecule at room temperature. The use of fluorophenyl resins in the solid phase synthesis of amides, peptides, hydroxamic acids, amines, urethanes, carbonates, sulfonamides, and alpha-substituted carbonyl compounds has been described in International Publication No. WO 99/67228 to Clerc et al.

1-Ethyl-3-(3-dimethylaminopropyl)carbodiimide/EDC/EDAC/EDCI/water soluble carbodiimide mediated coupling is currently the major mode of covalent immobilization of biomolecules to solid surfaces as described by Hermanson, G. T., in Bioconjugate Techniques, Academic Press, NY, 1996; Frey, A. et al., Bioconjugate Chem., 1999, 10, 562-571; Gilles, M. A. et al., Anal. Biochem., 1990, 184, 244-248; Chan V. W. F. et al., Biochem. Biophys. Res. Communications, 1988, 151(2), 709-716; and Valuev, I. L. et al., Biomaterials, 1998, 19, 41-43. The most frequently used method to immobilize biomolecules (such as oligonucleotides, proteins, and carbohydrates) onto fluorescent microspheres is by activating carboxy groups present on the surface of the microspheres. The activation requires excess N-ethyl-N′-(3-dimethylaminopropyl) carbodiimide (EDC) and a coupling pH of 4 to 6. The reaction between the carbodiimide and carboxyl functional groups forms an activated O-acylurea derivative reaction intermediate. A subsequent nucleophilic attack of the reaction intermediate by the primary nitrogen of the amino-groups of the biomolecule being attached to the microspheres releases the substituted urea and produces an amide linkage between the reaction intermediate and the biomolecule.

There are, however, a number of disadvantages to such activation of the carboxy groups. For example, the reaction intermediate has an extremely short half-life and rapidly undergoes hydrolysis or rearranges to produce the N-acylurea adduct. In addition, the optimum pH for the formation of O-acylurea is about 4-5. However, the primary amino group of the nucleophile is predominantly protonated at a pH of about 4-5 and is thus mostly unreactive. These limitations of the reaction intermediate can severely restrict coupling yields of biomolecules to microspheres.

Covalent coupling reagents such as N-hydroxysulfosuccinimide (NHS)/sulfo-NHS or carbodiimide based reactions oxidize the polysaccharides/protein structure at specific locations which alters the native state of polysaccharides. Coupling reagents are observed to be toxic and require lengthy and difficult laboratory practices, especially to comply with good manufacturing practices. “9.2 Premises and equipment” in Annex 2, TRS No 999 of “WHO good manufacturing practices for biological products” requires documented quality risk management of additional product in manufacturing facility, including potency and toxicological evaluation on cross-contamination risks. In some instances, standard EDC/sulfo-NHS coupling procedures may be somewhat problematic. For example, EDC and sulfo-NHS are hygroscopic solids that react with moisture in the air, and special precautions must be used to keep the surface modifier in the bottle fresh. Working solutions of the surface modifiers must be made immediately before use. The urea side products from EDC activation are sometimes hard to remove from the bead suspension and can interfere with subsequent coupling reactions or assays.

Some covalent coupling methods include manual amine coupling method that use 2 step carbodiimide reaction that chemically modifies the polysaccharide. Such methods require usage of cross-linking agents like cyanuric chloride and requires freshly prepared reagents. More over the reagents and chemicals used are considered toxic/hygroscopic.

Further, microsphere/bead coupling involving use of toxic chemistries also limits the use of technologies for automating the process of coupling (for example use of robotic liquid handling systems) for increasing throughput of laboratories.

Covalent reaction methods are reported to be variable and lab to lab reproducibility of results is difficult.

IN276304 (Serum Institute of India Private Limited) discloses a method for simultaneously detecting the presence of multiple anti-polysaccharide antibodies in a single test sample. The invention discloses use of four different chemistries (Poly-L-Lysine, EDC-ADH, DMTMM-NH2 and DMTMM-COOH) for coupling of each of pneumococcal polysaccharides to the carboxylated microspheres. Thus, it discloses use of conventional chemical coupling methods for Streptococcus polysaccharides.

IN491469 (Serum Institute of India Private Limited) discloses modified Sandwich ELISA for determining the antigen content and percent adsorption. The modified sandwich ELISA uses optimized parameters to quantify conjugated polysaccharide in the presence of 9 other conjugated antigens in a 10 valent vaccine.

Modified Amine Coupling of Pneumococcal polysaccharides to Beads (using EDC/NHS) (particularly for 6B, 9V and 19A polysaccharide) for preparing pneumococcal polysaccharide coupled beads is known.

Pickering et al., 2002; Lal et al., 2005; Whitelegg et al., 2012 describe a PLL (poly-L-lysine) conjugation reaction. Biagini et al., 2003 described conventional chemistry method based on sodium periodate oxidation/ADH. Schlottmann et al., 2006 provide for DMTMM (4-(4,6-Dimethoxy-1,3,5-triazin-2-yl)-4-methylmorpholinium chloride) based method. Covalent coupling reactions are indicated to be associated with limitations. The reactions oxidize the saccharide structure which alters the native state of polysaccharide. The coupling of DMTMM to microspheres performs well, but the microspheres coupled to DMTMM are more hydrophobic than microspheres activated with the commonly used surface modifier, sulfo-N-hydroxysuccinimide (sulfo-NHS). In other words, the DMTMM modified microspheres exhibited a propensity to stick to each other rather than dispersing in an aqueous solution. In contrast, microspheres with sulfo-NHS groups attached thereto retain a water-loving (i.e., hydrophilic) group (the sulfo) on the surface thereof when the sulfo-NHS is reacted with the original carboxyl group on the microspheres. The microspheres, therefore, stay well dispersed in water and aqueous solutions and solvents. In contrast, DMTMM is soluble in water because of the quaternary ammonium salt moiety that it contains. After reaction with a carboxyl group on the surface of a microsphere, this positive charge is lost due to its solubility in water. In this manner, hydrophilic carboxyl groups on the surface of the microsphere are replaced with hydrophobic aromatic rings thereby reducing the hydrophilicity of the microspheres.

WHO mentions (for e.g. WHO TRS 977 in case of Pneumococcal Vaccines) that for polysaccharide protein conjugate vaccines, antigen content determination is crucial as a good manufacturing practice.

Thus, it would be advantageous to develop a method for altering the surface characteristics of a microsphere without one or more of the disadvantages described above. There is an unmet need for a method/process to couple microsphere to saccharides that is rapid, simple, repeatable, cost effective, scalable, nontoxic and provides stable saccharide coupled microspheres with structure of saccharides remaining intact/unaffected.

### OBJECTS OF THE DISCLOSURE

Some of the objects of the present disclosure, which at least one embodiment herein satisfies, are as follows:

An object of the present disclosure is to provide a rapid, simple, repeatable, cost effective, scalable, non-toxic method/process to couple microsphere to saccharides to obtain stable saccharide couple microsphere that overcomes the drawbacks associated with previously reported conventional/chemical coupling methods.

Another object of the present disclosure is to provide an improved method of coupling that does not modify the structure of saccharides and is applicable for wide range of bacterial polysaccharides.

Yet another object of the present disclosure is to provide a method of non-covalent (electrostatic) coupling of polysaccharides to microspheres to form couples where structure of saccharides remains unaffected/intact and retains epitope confirmation.

Still another object of the present disclosure is to provide a method of coupling microsphere with streptococcal saccharides.

Yet another object of the present disclosure is to provide a method of coupling microsphere with meningococcal saccharides.

Still another object of the present disclosure is to provide a method of coupling microsphere with Haemophilus saccharides.

Yet another object of the present disclosure is to provide a method of coupling microsphere with Salmonella saccharides.

Still another object of the present disclosure is to provide assays based on the saccharide coupled microspheres to determine potency, identity, immune response associated with the saccharides.

Other objects and advantages of the present disclosure will be more apparent from the following description, which is not intended to limit the scope of the present disclosure.

### SUMMARY OF THE DISCLOSURE

The present invention provides a rapid, simple, cost effective, scalable method of coupling microspheres with saccharides to obtain the saccharide coupled microspheres which are used to determine antigen content, identity, free polysaccharide estimation, and antibody concentration.

The present invention is directed to a method of non-covalent (electrostatic) coupling polysaccharides to microspheres to form couples under specific reaction conditions (specific size and concentration of polysaccharides, specific chemical composition) where the structure of polysaccharides remains unaffected. Beads surfaces are activated using bead reagent, wherein the bead reagent includes metal ions. The activated beads are used for the coupling of saccharides using coupling buffer for specific incubation temperature and time. The prepared bead mixture is utilized in pneumococcal, Hib vaccine development assays such as antigen content determination, identity assay, free Ps estimation in drug product, estimation of antibody concentration (IgG) in clinical (human) sera samples, estimation of IgG titer in animal sera sample and the like.

Accordingly, in one aspect, the present invention is directed to a method of coupling a saccharide to a microsphere, the method comprising:
a. providing the microsphere;
b. providing the saccharide;
c. diluting the saccharide with a buffer at pH in range of 3.0 to 9.0;
d. mixing the microsphere with the saccharide to form the saccharide coupled microsphere;

wherein the mixing further includes incubation at temperature in range of 20° C. to 40° C. for incubation time in range of 30 mins to 180 mins.

In accordance with the embodiments of the present invention,
the mixing is performed with the coupling ratio of the microsphere to the saccharide in range of 50 to 12500,
the microsphere is activated with a bead reagent before mixing with the saccharide, wherein the bead reagent includes metal ions, and
optionally, the saccharide coupled microsphere is formed by non-covalent electrostatic coupling.

In an embodiment of the present invention, the buffer includes phosphate buffered saline with tween (PBST) buffer, (4-(2-hydroxyethyl)-1-piperazineethane sulfonic acid (HEPES) buffer, tris-aminomethane (Tris) buffer, 2-(N-morpholino)ethane sulfonic acid (MES) buffer, and 3-(N-morpholino) propane sulfonic acid (MOPS) buffer.

In an embodiment of the present invention, the saccharide is bacterial saccharide including Group A Streptococcus, Group B Streptococcus, Streptococcus pneumoniae/pneumococcus, Haemophilus bacteria, Haemophilus influenzae bacteria, Haemophilus influenzae type b bacteria (Hib), Salmonella, Typhoidal salmonella, Non-typhoidal salmonella, Salmonella typhi, Salmonella typhimurium, Salmonella paratyphi, Streptococcus pyogenes, Streptococcus agalactiae, Shigella, Shigella dysenteriae, Shigella flexneri, Shigella sonnei, Escherichia coli, or Neisseria meningitidis/meningococcus saccharide.

In an embodiment of the present invention, the saccharide is Streptococcus pneumoniae saccharide,
wherein the Streptococcus pneumoniae saccharide is selected from serotypes 1, 2, 3, 4, 5, 6, 6A, 6B, 6C, 6D, 7, 7B, 7C, 7F, 8, 9, 9A, 9L, 9N, 9V, 10, 10A, 10B, 10C, 10F, 10X, 11, 11A, 11B, 11C, 11F, 12, 12A, 12B, 12F, 13, 14, 15, 15A, 15B, 15BC, 15C, 15F, 16, 16F, 17, 17A, 17F, 18, 18A, 18B, 18C, 18F, 19, 19A, 19B, 19F, 20, 20A, 20B, 20F, 21, 22, 22A, 22F, 23, 23A, 23B, 23F, 24, 24F, 25, 25F, 26, 27, 28, 28A, 28F, 29, 30, 31, 32, 33, 33A, 33B, 33C, 33D, 33F, 34, 35, 35A, 35B, 35C, 35D, 35F, 36, 37, 38, 39, 40, 41, 42, 43, 44, 45, 46, 47, and/or 48, and
wherein the Streptococcus pneumoniae saccharide has a molecular size in range of 50 kDa to 3000 kDa, is diluted with PBST buffer at a pH in range of 3.0 to 9.0 and has concentration of 0.05 mg/mL to 50.0 mg/mL.

In an embodiment of the present invention, the Streptococcus pneumoniae saccharide is mixed with the microsphere and incubated at temperature of 23° C. to 39° C., for incubation time of 60 mins to 120 mins.

In an embodiment of the present invention, the saccharide is Haemophilus influenzae type b bacteria (Hib) saccharide,
wherein the Haemophilus influenzae type b bacteria (Hib) saccharide has a molecular size in range of 0.05 kDa to 5 kDa, is diluted with PBST buffer at a pH in range of 4 to 6 and has concentration of 1.0 μg/mL to 50.0 μg/mL.

In an embodiment of the present invention, the Haemophilus influenzae type b bacteria (Hib) saccharide is mixed with the microsphere and incubated at temperature of 20° C. to 30° C., for incubation time of 60 mins to 120 mins.

In an embodiment of the present invention, the saccharide is Neisseria meningitidis saccharide serotypes selected from meningococcal serotypes A (type I and III), B (type II), B6, B16, C (type II-alpha), D (type IV), Z′/E, E29, H, I, K, K454, L, M, W135, X, Y, Z, and wherein the Neisseria meningitidis polysaccharide has a molecular size in range of 75 kDa to 3000 kDa, is diluted with PBST buffer at a pH in range of 4 to 7 and has concentration of 0.01 mg/mL to 10.0 mg/mL.

In an embodiment of the present invention, the Neisseria meningitidis polysaccharide is mixed with the microsphere and incubated at temperature of 20 to 40° C., for incubation time of 60 mins to 120 mins.

Accordingly, in one aspect, the present invention is directed to a method of coupling a saccharide to a microsphere, the method comprising:
a. providing the microsphere;
b. providing the saccharide;
c. diluting the saccharide with the buffer at pH in range of 3.0 to 9.0; wherein and the saccharide is in range of 0.1 mg/ml to 50.0 mg/ml;
d. mixing the microsphere with the saccharide to form the saccharide coupled microsphere;
wherein the saccharide has a size in the range of 50.0 kDa to 3000 kDa; and
wherein the mixing includes incubation at temperature in range of 20° C. to 40° C. for incubation time in range of 30 mins to 180 mins.

In another aspect, the present invention is directed to the saccharide coupled microsphere obtained by the method as disclosed herein.

In an embodiment of the present invention, the saccharide coupled microsphere has Mean Florescence intensity/MFI value in the range of 200 to 20000.

In an embodiment of the present invention, the saccharide coupled microsphere is a Streptococcus pneumoniae saccharide coupled microsphere.

In an embodiment of the present invention, the saccharide coupled microsphere is a Haemophilus influenzae type b bacteria (Hib) saccharide coupled microsphere.

In an embodiment of the present invention, the saccharide coupled microsphere is a Neisseria meningitidis saccharide coupled microsphere.

In another aspect, the present invention is directed to a method of evaluating immunogenicity of immunogenic composition, the method comprising:
a. providing a test sample corresponding to the saccharide in the immunogenic composition;
b. providing the saccharide coupled microsphere corresponding to the saccharide in the immunogenic composition; and
evaluating the immunogenicity of the immunogenic composition by antigen content determination, or estimating antibody concentration (IgG), or identity assay, free polysaccharide estimation using the test sample and the saccharide coupled microsphere,
wherein the saccharide coupled microsphere is obtained by the method of the present disclosure.

In another aspect, the present invention is directed to an antigen content determination method using the saccharide coupled microsphere obtained by the method as disclosed herein.

In another aspect, the present invention is directed to an identity assay method using the saccharide coupled microsphere obtained by the method as disclosed herein.

In another aspect, the present invention is directed to a free saccharide estimation method using the saccharide coupled microsphere obtained by the method as disclosed herein.

In another aspect, the present invention is directed to a method of estimating antibody concentration (IgG) in sera sample using the saccharide coupled microsphere obtained by the method as disclosed herein (human/animal).

In another aspect, the present invention is directed to the saccharide coupled microsphere to determine antibody titre of an immunogenic composition/a vaccine.

In another aspect, the present invention is directed to apparatus comprising the saccharide coupled microsphere.

### BRIEF DESCRIPTION OF THE ACCOMPANYING DRAWING

The present disclosure will now be described with the help of the accompanying drawing, in which:

FIGS. 1a and 1b illustrates an embodiment with Streptococcus pneumoniae polysaccharide coupled microsphere with details of MFI value against Type 1 serotype samples;

FIGS. 2a and 2b illustrates an embodiment with Streptococcus pneumoniae coupled microsphere with details of MFI value against Type 2 serotype samples;

FIGS. 3a and 3b illustrates an embodiment with Streptococcus pneumoniae coupled microsphere with details of MFI value against Type 4 serotype samples;

FIGS. 4a and 4b illustrates an embodiment with Streptococcus pneumoniae coupled microsphere with details of MFI value against Type 7F serotype samples;

FIGS. 5a and 5b illustrates an embodiment with Streptococcus pneumoniae polysaccharide coupled microsphere with details of MFI value against Type 8 serotype samples;

FIGS. 6a and 6b illustrates an embodiment with Streptococcus pneumoniae polysaccharide coupled microsphere with details of MFI value against Type 10A serotype samples;

FIGS. 7a and 7b illustrates an embodiment with Streptococcus pneumoniae polysaccharide coupled microsphere with details of MFI value against Type 24F serotype samples;

FIG. 8a to 8q illustrate embodiments with details of MFI value against saccharide coupled microspheres with saccharides of pneumococcal serotypes 3, 5, 6A, 6B, 9V, 10A, 11A, 12F, 14, 15B, 18C, 19A, 19F, 22F, 23F, 24F, 33F, respectively;

FIG. 9 illustrate an embodiment with details of MFI value against saccharide coupled microspheres with saccharides of Haemophilus influenzae type b; and

FIG. 10a to 10e illustrate embodiments with details of MFI value against saccharide coupled microspheres with saccharides of Neisseria meningitis serotypes (A, C, W, X, Y respectively).

### DETAILED DESCRIPTION OF THE DISCLOSURE

Before the present compositions and formulations of the invention are described, it is to be understood that this invention is not limited to particular compositions and formulations described, since such compositions and formulation may, of course, vary. It is also to be understood that the terminology used herein is not intended to be limiting, since the scope of the present invention will be limited only by the appended claims.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”. More specifically, the term “comprise” as used herein means that the claim encompasses all the listed elements or method steps, but may also include additional, unnamed elements or method steps. For example, a method comprising steps a), b) and c) encompasses, in its narrowest sense, a method which consists of steps a), b) and c). The phrase “consisting of” means that the composition (or device, or method) has the recited elements (or steps) and no more. In contrast, the term “comprises” can encompass also a method including further steps, e.g., steps d) and e), in addition to steps a), b) and c).

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(A)”, “(B)” and “(C)” or “(a)”, “(b)”, “(c)”, “(d)”, “i”, “ii” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may do. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art.

Furthermore, the ranges defined throughout the specification include the end values as well, i.e. a range of 1 to 10, between 1 to 10 imply that both 1 and 10 are included in the range. For the avoidance of doubt, the applicant shall be entitled to any equivalents according to applicable law.

As used herein, the term “about” when qualifying a value of a stated item, number, percentage, or term refers to a range of plus or minus 10 percent, 9 percent, 8 percent, 7 percent, 6 percent, 5 percent, 4 percent, 3 percent, 2 percent or 1 percent of the value of the stated item, number, percentage, or term. Preferred is a range of plus or minus 10 percent.

In case numerical ranges are used herein such as “in a concentration between 1 and 5 micromolar”, the range includes not only 1 and 5 micromolar, but also any numerical value in between 1 and 5 micromolar, for example, 2, 3 and 4 micromolar. The term “in vitro” as used herein denotes outside, or external to, the animal or human body. The term “in vitro” as used herein should be understood to include “ex vivo”. The term “ex vivo” typically refers to tissues or cells removed from an animal or human body and maintained or propagated outside the body, e.g., in a culture vessel. The term “in vivo” as used herein denotes inside, or internal to, the animal or human body.

The term “vaccine” is optionally substitutable with the term “immunogenic composition” and vice versa.

The term “microsphere” is optionally substitutable with the term “bead” and vice versa.

The terms microsphere-polysaccharide conjugation and microsphere-polysaccharide coupling are interchangeably used throughout the specification.

### Definitions

The term “saccharide” throughout this specification may indicate polysaccharide, saccharide or oligosaccharide, or combinations thereof. The capsular saccharide antigen may be a full-length polysaccharide or it may be extended to bacterial ‘sized-saccharides’ and ‘oligosaccharides’ (which naturally have a low number of repeat units, or which are polysaccharides reduced in size for manageability, but are still capable of inducing a protective immune response in a host).

The term “MFI” is associated with Mean Fluorescence Intensity.

Other “biomolecules” which can be conjugated to polysaccharide (interchangeably referred to as PSs) include enzymes, enzyme substrates, enzyme inhibitors, hormones, antibiotics, antibodies, antigens, peptides, polypeptides, proteins, other polysaccharides, nucleic acids, nucleosides, nucleotides, polynucleotides, and the like.

The terms “covalent” and “valence” refer to a chemical bond between two atoms in a molecule created by the sharing of electrons, usually in pairs, by the bonded atoms and may involve single bonds or multiple bonds. The term “covalent” does not include hydrophobic/hydrophilic interactions, hydrogen-bonding, and van der Waals interactions.

The term “non-covalent” refers to interactions between two or more molecules and/or by two or more parts of the same molecule which are not “covalent” in nature. Such “non-covalent” interactions include electrostatic interactions such as, hydrogen bonds, hydrophobic/hydrophilic interactions, salt bridges, and van der Waals interactions.

The term “coating” as used herein refers to non-covalent immobilization of polysaccharides on solid surfaces, e.g., through adsorption. The nature of passive adsorption predominantly involves multiple hydrophobic interactions between solid phase and the polysaccharide.

The terms “immunogen” and “immunogenic” refer to substances capable of producing or generating an immune response in an organism directed specifically against the polysaccharide. The terms “antigenic” and “antigenicity” refer to the capability of a polysaccharide/saccharide to be specifically bound by an antibody to the polysaccharide.

The term “immunospecific” means that the antibodies corresponding to the polysaccharide/saccharide antigens exhibit a substantially greater affinity for the PSs attached to solid supports and biomolecules compared to the affinity for other antigens. It is also generally desirable that the affinity of antibodies corresponding to the polysaccharide/saccharide antigens toward PSs attached to solid supports and biomolecules is similar to that toward the corresponding unattached PSs.

### DISCLOSURE

### Method of Coupling

In one aspect, the present invention is directed to a method of coupling a saccharide to a microsphere to obtain a saccharide coupled microsphere, the method comprising:
a. providing the microsphere;
b. providing the saccharide;
c. diluting the saccharide with a buffer at pH in range of 3.0 to 9.0;
d. mixing the microsphere with the saccharide to form the saccharide coupled microsphere;
wherein the mixing further includes incubation at temperature in range of 20° C. to 40° C. for incubation time in range of 30 mins to 180 mins.

In one aspect, the present invention is directed to a method of coupling a saccharide to a microsphere, the method comprising:
a. providing the microsphere;
b. providing the saccharide;
c. diluting the saccharide with the buffer at pH in range of 3.0 to 9.0; wherein and the saccharide is in range of 0.1 mg/ml to 50.0 mg/ml;
d. mixing the microsphere with the saccharide to form the saccharide coupled microsphere;
wherein the saccharide has a size in the range of 50.0 kDa to 3000 kDa; and
wherein the mixing further includes incubation at temperature in range of 20° C. to 40° C. for incubation time in range of 30 mins to 180 mins.

The present invention pertains to the method for coupling saccharides to microsphere such that the non-conventional saccharide to bead coupling uses metal activated beads and the coupling is non-covalent electrostatic coupling.

The present invention pertains to the method for coupling saccharides to microsphere using non-covalent coupling of saccharides to microspheres to form the saccharide coupled microsphere under specific reaction conditions wherein the structure of polysaccharides remains intact/unaffected and retains epitope confirmation.

The present invention pertains to the method for coupling saccharides which is based on principles of metal coordination chemistry and electrostatic mode of interaction-based coupling of antigens wherein bead surface is first activated using metal solutions of Nickel to introduce suitable metal ions on the bead surface.

The saccharide coupled microsphere obtained by the method of present invention finds application developing immunochemical assays for
a) determining Antigen content,
b) identity assays,
c) determining antibody/IgG or serology applications,
d) estimating stability of saccharides or estimating free saccharide content,
e) other serological analysis.

The method has optimized a specific combination of concentration of polysaccharide, size of polysaccharide, reaction time, temperature, pH of buffer/PBST to allow development of electron donor sites which allow electrostatic attachment of Ps on to activated beads structure that too with polysaccharide structure remaining intact.

In an embodiment, the present invention pertains to the method of coupling saccharides to microspheres, the method comprising:
a. providing at least one microsphere;
b. providing at least one saccharide; and
c. mixing the at least one microsphere with the at least one saccharide to form a saccharide coupled microsphere;
wherein the saccharide has a size in the range of 0.01 kDa to 3000 kDa,
optionally wherein the at least one microsphere includes at least one activated microsphere and
optionally wherein the coupling of saccharides to microsphere is non-covalent electrostatic coupling.

In an embodiment, the method includes providing at least one or more microsphere.

In an embodiment, the method includes providing at least one or more saccharide.

In an embodiment, the method includes mixing the at least one or more microspheres with the at least one or more saccharides to form a saccharide coupled microsphere.

In an embodiment of the present disclosure, the method comprises
mixing is performed with the coupling ratio of the microsphere to the saccharide is in range of 50 to 12500,
the microsphere is activated with a bead reagent before mixing with the saccharide, wherein the bead reagent includes metal ions, and
optionally, the saccharide coupled microsphere is formed by non-covalent electrostatic coupling.

### Providing Microsphere

In an embodiment, the method of coupling saccharides to microspheres includes step of providing at least one microsphere.

### Microspheres

Microspheres, microparticles, microcapsules, polymer particles and beads, referred to herein collectively as “microspheres”, are solid or semi-solid particles. In an embodiment, the microsphere has a diameter of less than one millimetre, more preferably less than 100 microns, which can be formed of a variety of materials, including synthetic polymers, proteins, and polysaccharides. Microspheres are used for separations, diagnostics, and drug delivery. Microspheres for separations techniques are formed of polymers of synthetic or protein origin, such as polyacrylamide, hydroxyapatite or agarose. Polymeric microspheres are used to separate molecules including proteins based on molecular weight and/or ionic charge or by interaction with molecules chemically coupled to the microparticles. In the diagnostic area, microspheres are frequently used to immobilize an enzyme, substrate for an enzyme, or labelled antibody, which is then interacted with a molecule to be detected, either directly or indirectly. In the controlled drug delivery area, molecules are encapsulated within microparticles or incorporated into a monolithic matrix for subsequent release.

Microspheres have been commercially available as a tool for biochemists for many years. For example, antibodies conjugated to beads create relatively large particles specific for particular ligands. The large antibody-coated particles are routinely used to crosslink receptors on the surface of a cell for cellular activation, are bound to a solid phase for immunoaffinity purification, and may be used to deliver a therapeutic agent that is slowly released over time, using tissue or tumour-specific antibodies conjugated to the particles to target the agent to the desired site.

A number of different techniques are routinely used to make these microspheres from synthetic polymers, natural polymers, proteins and polysaccharides, including phase separation, solvent evaporation, emulsification, and spray drying. Examples of suitable polymers for the formation of microspheres include polystyrenes, polyesters, polyethers, polyolefins, polyalkylene oxides, polyamides, polyacrylates, polymethacrylates, polyurethanes, celluloses, polyisoprenes, silica, and polysaccharides, particularly cross-linked polysaccharides, such agarose, which is available as Sepharose, dextran, available as Sephadex and Sephacryl, cellulose, starch, and the like. Exemplary polymers used are addition polymers, such as polystyrene, polyvinyl alcohol, homopolymers and copolymers of derivatives of acrylate and methacrylate, particularly esters and amides having free hydroxyl functionalities. However, the availability and cost of these other polymeric particles make the use of polystyrene particles preferred. Other considerations which favour polystyrene are uniformity in the size and shape of the particles which are to be conjugated. The size of the polymer particles ranges from about 0.1 to about 100.0 μm. The preferred particle size is in the range of about 0.5 to 20.0 μm.

Other polymers used for the formation of microspheres include (a) homopolymers and copolymers of lactic acid and glycolic acid (PLGA) as described in U.S. Pat. No. 5,213,812 to Ruiz; U.S. Pat. No. 5,417,986 to Reid et al.; U.S. Pat. No. 4,530,840 to Tice et al.; U.S. Pat. No. 4,897,268 to Tice et al.; U.S. Pat. No. 5,075,109 to Tice et al.; U.S. Pat. No. 5,102,872 to Singh et al.; U.S. Pat. No. 5,384,133 to Boyes et al.; U.S. Pat. No. 5,360,610 to Tice et al.; and European Patent Application Publication Number 248,531 to Southern Research Institute; (b) block copolymers such as tetronic 908 and poloxamer 407 as described in U.S. Pat. No. 4,904,479 to Ilium; and (c) polyphosphazenes as described in U.S. Pat. No. 5,149,543 to Cohen et al.

Microspheres may be of a latex type. The term “latex,” as used herein, pertains to a stable colloidal dispersion of a polymeric substance in an aqueous medium. “Latex” is intended to mean an emulsion consisting substantially of latex mixed with water as a medium, but may also include additional ingredients such as bulking agents, fixing agents, adhesives, dyes and plasticizers, such latex compounds requiring heating to remove moisture and ensure effective adhesion. Also considered within the scope of the present invention are embodiments wherein the dispersion medium comprises an organic solvent. The dispersed particles preferably have an average particle size of about 0.1-100 μm, more preferably about 0.5-20 μm. The particle size distribution of the dispersed particles is not particularly limited, and the particles may have either wide particle size distribution or monodispersed particle size distribution. The polymer latex used in the present invention may be latex of the so-called core/shell type other than ordinary polymer latex having a uniform structure. In this case, use of different glass transition temperatures of core and shell may be preferred.

The naturally occurring or synthetic latex polymers are preferably derived from one or more unsaturated monomers which are capable of polymerizing in an aqueous environment. Particularly preferred are the use of any of the following monomers: (meth)acrylic based acids and esters, acrylonitrile, styrene, divinylbenzene, vinyl esters including but not limited to vinyl acetate, acrylamide, methacrylamide, vinylidene chloride, butadiene and vinyl chloride. The polymers that are produced may take the form of homopolymers (i.e., only one type of monomer selected) or copolymers (i.e., mixtures of two or more types of monomer are selected; this specifically includes terpolymers and polymers derived from four or more monomers). In one form, the copolymer could be a random, a block, or an alternating copolymer. Crosslinking is useful in many polymers for imparting structural integrity and rigidity to the microparticle.

Latex microspheres can be based on a range of synthetic polymers, such as polystyrene, polyvinyltoluene, polystyrene-acrylic acid, polyacrolein, and poly(meth)acrylate esters and their copolymers. The monomers used are normally water-insoluble, and are emulsified in aqueous surfactant so that monomer droplets and/or micelles are formed, which are then induced to polymerize by the addition of initiator to the emulsion. Substantially spherical monodisperse polymer particles are produced. By controlling the conditions, a variety of size ranges can be provided.

Microspheres for use in conjugates and methods of coupling are commercially available. Microspheres include xMAP™ from Luminex Corporation (Austin, Tex.). xMAP™ microspheres are 5.6 μm in diameter and composed of polystyrene, divinylbenzene and methacrylic acid, which provides surface carboxylate functionality for covalent attachment of Saccharides and biomolecules. The microspheres include microsphere dyed with red- and/or infrared-emitting fluorochromes. By proportioning the concentrations of each fluorochrome, spectrally addressable microsphere sets are obtained. The microsphere sets are mixable, and are analysed using the Luminex100™ instrument (Luminex). Each set are identified and classified by a distinct fluorescence signature pattern.

When particles are used as the solid phase, one means of separating bound from unbound species is to use particles that are made of or that include a magnetically responsive material. Such a material is one that responds to a magnetic field. Magnetically responsive materials include paramagnetic materials, ferromagnetic materials, ferrimagnetic materials, and metamagnetic materials. In an embodiment, the metals include Nickel, Iron, Copper, Magnesium, Chromium, Zinc, cobalt, as well as metal oxides such as Fe₃O₄, BaFeI2O19, CoO, NiO, Mn₂O₃, Cr₂O₃, CoFe₂O₄, and CoMnP. The magnetically responsive material may constitute the entire particle. In an embodiment, the magnetically responsive material is one component of the particle, the remainder being a polymeric material to which the magnetically responsive material is affixed.

In an embodiment, the microsphere surface is activated using metal solutions of Nickel, wherein the suitable metal ions are introduced on the bead surface.

In an embodiment, the metals are used in the metal induced reaction.

The magnetically responsive materials are used and quantity of material is not critical and can vary over a wide range. The quantity can affect the density of the particle, however, and both the quantity and the particle size can affect the ease of maintaining the particle in suspension.

In an alternate embodiment, the concentration of magnetically responsive material is low enough to facilitate estimations/testing/assays. The magnetically responsive material in a particle is in range from 0.1% to 75.0% by weight of the particle as a whole, or in range of 2.0% to 50.0%, or in range of 3.0% to 25.0%, or in range of 5.0% to 15.0%. In another embodiment, the magnetically responsive material can be dispersed throughout the polymer, applied as a coating on the polymer surface or as one of two or more coatings on the surface, or incorporated or affixed in any other manner that secures the material in the polymer matrix.

In an embodiment, the microsphere for conjugation to a saccharide is a polymer selected from the group consisting of a polystyrene, a polyester, a polyether, a polyolefin, a polyalkylene oxide, a polyamide, a polyacrylate, a polymethacrylate and a polyurethane, or a mixture thereof. In a preferred embodiment, the microsphere is a polystyrene.

In an alternate embodiment, the microsphere contains carboxyl groups and includes both magnetic microspheres and non-magnetic microspheres.

In yet another alternate embodiment, the microsphere is coupled to a linker molecule prior to reacting the microsphere with the activated polysaccharide. In another embodiment, the linker compound is selected from the group consisting of α,ω-diaminoalkane, adipic acid dihydrazide and α,ω-alkanedihydrazide.

In an embodiment the microsphere includes specific concentration of internal dyes that correlate to a specific bead region. Internal dyes differ between regions, the outer coating of carboxyl groups is same across all bead regions.

In a preferred embodiment, the bead includes optically detectable beads, fluorescence beads. xMAP beads. The beads include magnetic beads and non-magnetic beads.

In a preferred embodiment, the microsphere is a magnetic microsphere.

The magnetic microspheres have mean microsphere diameter in range of 1 μm to 100 μm, or in range of 1 μm to 80 μm, or in range of 1 μm to 60 μm or in range of 1 μm to 40 μm or in range of 1 μm to 20 μm. In a more preferred embodiment the microsphere has mean microsphere diameter in range of 2 μm to 15 μm.

In another more preferred embodiment, the microsphere has mean microsphere diameter in range of 2 μm to 10 μm. In a more preferred embodiment, the microsphere is a magnetic bead microsphere that are fluorescently dyed magnetic microspheres. The magnetic bead microsphere act as both identifier and solid surface to build assay.

In another preferred embodiment, the microsphere is a non-magnetic microsphere. In a more preferred embodiment, the microsphere is a polystyrene microsphere.

The non-magnetic microspheres have a mean microsphere diameter are in range of 1 μm to 100 μm, or in range of 1 μm to 80 μm, or in range of 1 μm to 60 μm or in range of 1 μm to 40 μm or in range of 1 μm to 20 μm. In a more preferred embodiment the microsphere has mean microsphere diameter in range of 2 μm to 15 μm.

In another more preferred embodiment, the microsphere has mean microsphere diameter in range of 2 μm to 10 μm. In a more preferred embodiment, the microsphere is a non-magnetic microsphere that is internally labelled with fluorescent dyes and contains surface carboxyl groups.

In an embodiment, the microspheres are carboxylated microparticles (“beads”) that are color-coded into 100 spectrally distinct sets, or “regions.” Each of these bead regions are distinguishable by an xMAP® instrument that performs interrogation of up to 100 different analytes simultaneously from a single sample.

In a preferred embodiment, the at least one microsphere provided is an activated microsphere.

In an embodiment, the blank beads/microspheres are provided. The bead/microsphere surface is activated using metals. In a more preferred embodiment, the at least one microsphere includes an activated magnetic microsphere.

In another embodiment, the bead/microsphere surface is activated using buffers.

### Bead Reagent

In an embodiment, the microspheres/beads are activated using bead reagent (hereinafter interchangeably referred to as the activation buffer).

In an embodiment, the method of coupling the microspheres with the saccharide includes providing at least one microsphere, wherein the at least one microsphere is activated.

The bead reagent activates bead surfaces forming a co-ordination bond with metal ions. The bead reagent facilitates metal ions to bind the microsphere. The role of bead reagent is to activate the blank beads under an incubation period. The activated beads are used for the coupling of saccharides.

In an embodiment the bead reagent includes solutions with at least one divalent cation, trivalent cation, or tetravalent cations including metals or their metal oxides. In another embodiment, the bead reagents include at least one divalent cation. In another embodiment, the bead reagents include gold, silver, nickel, iron, copper, magnesium, chromium, zinc, cobalt, Cd²+, Co²+, Ni²+, Pb²+, Zn²+, Ca²+, Cr²+, as well as metal oxides such as Fe₃O₄, BaFeI2O19, CoO, NiO, Mn₂O₃, Cr₂O₃, CoFe₂O₄, and CoMnP. In a preferred embodiment, the metal ions include nickel ions, chromium ions, and gold ions.

In a more preferred embodiment, the metal ions in the bead reagent are chromium ions.

In another more preferred embodiment, the at least one microsphere is activated using the buffer reagent/the activation reagent. The buffer reagent includes Chromium perchlorate, 2-(N-morpholino) ethane sulphonic acid, sodium chloride, polypropylene/polyethlylene glycol copolymer, isothiazolinones, and water.

In an embodiment, the bead reagent includes Anteotech regent A-CMPARA1. The microsphere is activated with the bead reagent.

In an embodiment, the at least one microsphere is activated using the bead reagent or the activation buffer for time in range of 30 mins to 90 mins.

In a preferred embodiment, the at least one microsphere is activated using the bead reagent for time in range of 30 mins to 80 mins, or in range of 30 mins to 70 mins.

In another preferred embodiment, the at least one microsphere is activated using the bead reagent for time in range of 40 mins to 70 mins, in range of 50 mins to 70 mins.

In a more preferred embodiment, the at least one microsphere is activated using the bead reagent for 60 mins.

In a preferred embodiment, the at least one microsphere provided is an activated microsphere activated using the bead reagent or the activation reagent. In a more preferred embodiment, the at least one microsphere includes an activated magnetic microsphere activated using the bead reagent or the activation reagent.

### Providing Saccharides

In an embodiment, the method includes providing at least one saccharide. Saccharide herein after is interchangeably referred to as oligosaccharide, polysaccharides, short chained saccharides, capsular saccharides.

In an embodiment, the present invention is directed to the method of coupling saccharides to microspheres, wherein the method includes step of providing at least one saccharide.

In another embodiment, this invention provides a method for coupling a saccharide to a microsphere or a biomolecule.

The saccharide includes immunogenic and/or antigenic polysaccharides. The method for coupling a saccharide to a microsphere or a biomolecule does not change the immunogenicity and/or the antigenicity of the polysaccharide.

In another embodiment, the saccharide includes capsular polysaccharides, polysaccharides/saccharides derived from lipopolysaccharides (LPS) and lipooligosaccharides (LOS) of Gram-negative bacteria cell-wall, such as the O-specific side chain, and also fungal cell-wall polysaccharides.

Polysaccharides are composed of repeat units. For use in conjugates of the invention, in certain embodiments a saccharide comprises at least about 4 repeat units preferably up to about 3,000. Thus, the number-average degree of polymerization (the average number of glycose rings contained in one molecule) of the saccharide is at least about 4, with no particular upper limit, though it is preferably at most about 3,000. Especially for use as in an immunoassay, the number-average degree of polymerization of a saccharide is between 4 to 1,000, and particularly between 4 to 700, and more particularly between 50 to 200.

In an embodiment, the saccharide has no repeat units.

In another embodiment the saccharide has repeat units. A repeat unit is characteristic of a given saccharide and thus the composition and molecular weight of the repeat unit greatly vary from one polysaccharide/saccharide to another. For example, while the repeat unit of most capsular polysaccharides/saccharides contains hydroxyl, carboxyl, and/or phosphoryl groups, some polysaccharides/saccharides also contain amino groups (e.g. Streptococcus pneumoniae serotype 1), whereas others do not (e.g. Streptococcus pneumoniae serotype 14) and some contain N-acetyls (e.g. Streptococcus pneumoniae serotype 14), whereas others do not (e.g. Streptococcus pneumoniae serotype 6B). Also, as a matter of example, the molecular weight of capsular polysaccharides of Streptococcus pneumoniae serotypes 3 and 4 is 360 and 847, respectively. Thus, there is no general correspondence between the number of repeat units and the molecular weight of the polysaccharide that may be globally applied, irrespective of the polysaccharide composition. In an embodiment, the molecular weight saccharide is in the average range of 1,000 to 5,500,000 Daltons. The molecular weight of a saccharide is expressed as a mean value, since a polysaccharide/saccharide is constituted by a population of molecules of heterogeneous size.

Polysaccharides may be either chemically synthesized, purified from a natural source according to conventional methods, or natural PSs can be further chemically modified. For example, in the case of bacterial or fungal polysaccharides, these latter may be extracted from the microorganisms and treated to remove the toxic moieties, if necessary. A particularly useful method is described by Gotschlich et al., J. Exp. Med., 129: 1349 (1969).

Polysaccharides may be used as synthesized or purified. They may be also depolymerized prior to use. Indeed, native capsular polysaccharides usually have a molecular weight greater than 10,000 Daltons. When it is preferred to use capsular polysaccharides of lower molecular weight, e.g. 10,000 to 20,000 Daltons on average, polysaccharides as purified may be submitted to fragmentation. To this end, conventional methods are available. For example, WO 93/07178 describes a fragmentation method using an oxidation-reduction depolymerization reaction.

The term “polysaccharide” as used herein is meant to include compounds made up of many hundreds or even thousands of monosaccharide units per molecule. These units are held together by glycosidic linkages. Their molecular weights are normally greater than about 5,000 and can range up to millions of Daltons. They are normally naturally-occurring, such as, for example, starch, glycogen, cellulose, gum arabic, agar, and chitin. The polysaccharide should have one or more reactive functional groups, such as hydroxyl, carboxyl, amino, phosphoryl, etc. The polysaccharide may be straight or branched chain.

The hydroxyl, carboxyl, phosphoryl, or amino groups of the polysaccharide that are involved in the linkage may be native functional groups. Alternatively, they may have been introduced artificially by chemical modification. Amino groups may be generated by controlled acidic or basic hydrolysis of native N-acyl groups such as N-acetyl groups. Hydrazide groups may be introduced by coupling the polymer with a linker, such as, e.g., adipic acid dihydrazide using conventional EDC-mediated coupling chemistry or other suitable means.

Polysaccharides that can be covalently linked according to methods described herein include starch-like and cellulosic material, but the present method is especially suitable for conjugating microbial polysaccharides that are haptens or immunogens. It is noted that the term “polysaccharides” as used herein comprises sugar-containing polymers and oligomers, whether they only contain glycosidic linkages or also phosphodiester or other linkages. They may also contain non-sugar moieties such as acid groups, phosphoryl groups, amino groups, hydroxyls and amino acids, and are optionally depolymerized.

Bacterial polysaccharides are described in details in Lennart Kenne and Bengt Lindberg, “Bacterial polysaccharides” in The polysaccharides, Vol. 2, Ed. G. O. Aspinall, 1983, Academic Press, pp. 287-363.

In another embodiment, the saccharide is a bacterial polysaccharide. In another embodiment, the bacterial polysaccharide is isolated from bacteria selected from the group consisting of Streptococcus spp., Streptococcus pyogenes, Streptococcus agalactiae, Streptococcus pneumoniae (pneumococcus, (1, 2, 3, 4, 5, 6, 6A, 6B, 6C, 6D, 6E, 6G, 6H, 7A, 7B, 7C, 7F, 8, 9A, 9L, 9F, 9N, 9V, 10F, 10B, 10C, 10A, 11A, 11F, 11B, 11C, 11D, 11E, 12A, 12B, 12F, 13, 14, 15A, 15C, 15B, 15F, 16A, 16F, 17A, 17F, 18, 18C, 18F, 18A, 18B, 19A, 19B, 19C, 19F, 20, 20A, 20B, 21, 22A, 22F, 23A, 23B, 23F, 24A, 24B, 24F, 25F, 25A, 27, 28F, 28A, 29, 31, 32A, 32F, 33A, 33C, 33D, 33E, 33F, 33B, 34, 35A, 35B, 35C, 35F, 36, 37, 38, 39, 40, 41F, 41A, 42, 43, 44, 45, 46, 47F, 47A, and 48), Streptococcus viridans, Salmonella spp., Salmonella typhi, Salmonella Paratyphi, Salmonella enteritidis, Salmonella Typhimurium, Neisseria meningitidis/meningococcus (serotypes such as A, B, B16, B6, C, D, E29, H, I, K, K454 L, M, W135, X, Y, and Z etc), Neisseria gonorrhoeae, Shigella spp., Haemophilus bacteria, Haemophilus influenzae bacteria (type a, b, c, or d), Haemophilus influenzae type b bacteria (Hib), Helicobacter pylori, Nontyphoidal salmonella, Chlamydia pneumoniae, Chlamydia trachomatis, Ureaplasma urealyticum, Mycoplasma pneumoniae, Staphylococcus spp., Staphylococcus aureus, Enterococcus faecalis, Bacillus anthracis, Vibrio cholera, Shigella spp (Shigella sonnei, Shigella flexneri, Shigella dysenteriae; Shigella boydii) Klebsiella pneumoniae, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospira spp., Hemophilus ducreyi, Corynebacterium diphtheria, Bordetella pertussis, Bordetella parapertussis, Bordetella bronchiseptica, Hemophilus influenzae, Escherichia coli, Erlichia spp., and Rickettsia spp and from fungi such as Candida albicans, Candida kefyr, Cryptococcus neoformans, Hansenula anomala, and Hansenula arabitolgens.

In an embodiment, the saccharide is bacterial saccharide including Group A Streptococcus, Group B Streptococcus, Streptococcus pneumoniae/pneumococcus, Haemophilus bacteria, Haemophilus influenzae bacteria, Haemophilus influenzae type b bacteria (Hib), Salmonella, Typhoidal salmonella, Non-typhoidal salmonella, Salmonella typhi, Salmonella typhimurium, Salmonella paratyphi, Streptococcus pyogenes, Streptococcus agalactiae, Shigella, Shigella dysenteriae, Shigella flexneri, Shigella sonnei, Escherichia coli, or Neisseria meningitidis/meningococcus saccharide.

The polysaccharide/saccharide is associated with bacteria, Streptococcus, Streptococcus pneumoniae/pneumococcus, Haemophilus bacteria, Haemophilus influenzae bacteria, Haemophilus influenzae type b bacteria (Hib), and Neisseria meningitidis/meningococcus.

In an embodiment, the saccharide is native or is modified. The modified saccharide includes size reduced saccharides. In an embodiment, the saccharide from stock is diluted with the buffer at a particular pH range corresponding to the saccharide and condition required for conjugation to the microsphere to obtain optimal MFI's to positive samples.

In an embodiment, the saccharide is a native saccharide.

In another embodiment, the saccharide is a size reduced saccharide. In another embodiment, the saccharide has a size in the range of 0.01 kDa to 3000 kDa.

In a preferred embodiment, the saccharide has a size in the range of 0.05 kDa to 2800 kDa, or 0.10 kDa to 2800 kDa, or 0.5 kDa to 2800 kDa, or 1.0 kDa to 2800 kDa, or 2.0 kDa to 2800 kDa, or 5.0 kDa to 2800 kDa, or 10.0 kDa to 2800 kDa, or 15.0 kDa to 2800 kDa, or 20.0 kDa to 2800 kDa, or 25.0 kDa to 2800 kDa, or 30.0 kDa to 2800 kDa, or 35.0 kDa to 2800 kDa, or 40.0 kDa to 2800 kDa, or 45.0 kDa to 2800 kDa, or 50.0 kDa to 2800 kDa.

In another preferred embodiment, the saccharide has a size in the range of 50.0 kDa to 2800 kDa, or 50.0 kDa to 2700 kDa, or 50.0 kDa to 2600 kDa, or 50.0 kDa to 2500 kDa, or 50.0 kDa to 2400 kDa, or 50.0 kDa to 2300 kDa, or 50.0 kDa to 2200 kDa, or 50.0 kDa to 2100 kDa, or 50.0 kDa to 2000 kDa, or 50.0 kDa to 1900 kDa, or 50.0 kDa to 1800 kDa, or 50.0 kDa to 1900 kDa, or 50.0 kDa to 1800 kDa, or 50.0 kDa to 1700 kDa, or 50.0 kDa to 1600 kDa, or 50.0 kDa to 1500 kDa, or 50.0 kDa to 1400 kDa, or 50.0 kDa to 1400 kDa, or 50.0 kDa to 1300 kDa, or 50.0 kDa to 1250 kDa.

In a more preferred embodiment, the saccharide has a size in the range of 50.0 kDa to 1200 kDa.

In a further more preferred embodiment, the saccharide has a size in the range of 50.0 kDa to 1150 kDa.

In a further more preferred alternate embodiment, the saccharide has a size in the range of 50.0 kDa to 1100 kDa.

In a further more preferred alternate embodiment, the saccharide has a size in the range of 50.0 kDa to 1000 kDa.

In a further more preferred alternate embodiment, the saccharide has a size in the range of 50.0 kDa to 800 kDa.

In a further more preferred alternate embodiment, the saccharide has a size in the range of 50.0 kDa to 600 kDa.

### Dilution of Saccharide

In an embodiment, the stock of native saccharide is diluted with a buffer prior to coupling.

### Buffer

In an embodiment, the saccharide is diluted with the buffer. The buffer acts as microsphere-polysaccharide coupling component at a pH and ion strength to enable efficient immobilization of polysaccharide to activated magnetic beads/microspheres.

In another embodiment, the present invention is directed to the method of coupling saccharides to microspheres, wherein the buffer includes phosphate-buffered saline with Tween 20 (PBST) buffer, (4-(2-hydroxyethyl)-1-piperazineethanesulfonic acid (HEPES) buffer, Tris-aminomethane (Tris) Buffer, 2-(N-morpholino)ethanesulfonic acid (MES) buffer, 3-(N-morpholino)propanesulfonic acid (MOPS) buffer, etc.

In another embodiment, the buffer used is phosphate buffer.

In a preferred embodiment, the buffer used is PBST buffer.

pH

In an embodiment, the present invention is directed to the method of coupling saccharides to microspheres, where the saccharide is diluted with a buffer at pH in range of 3.0 to 9.0.

In a preferred embodiment, the mixing is performed at pH in the range of 3.0 to 8.0, or in range of 3.0 to 7.5.

### Concentration of Saccharide

In another embodiment, the present invention is directed to the method of coupling saccharides to microspheres, wherein concentration of saccharide is in range of 0.1 mg/mL to 50.0 mg/mL.

In another embodiment, the present invention is directed to the method of coupling saccharides to microspheres, wherein concentration of saccharide is in range of 0.1 mg/mL to 40.0 mg/mL, or in range of 0.1 mg/mL to 30.0 mg/mL, or in range of 0.1 mg/mL to 20.0 mg/mL, or in range of 0.1 mg/mL to 10.0 mg/mL.

In an embodiment, the native saccharide (herein after referred interchangeably as PnPS) is provided in stock concentration range of 5 to 10 mg/ml. Size reduced saccharide is provided in stock concentration range of 11.0 to 15.0 mg/ml.

### Mixing

In an embodiment, the method includes mixing the at least one microsphere with the at least one saccharide to form a saccharide coupled microsphere.

In an embodiment, the method of coupling utilises metal induced reaction sites on surface of the bead/microsphere. The reaction sites create multivalent binding with target molecule/polysaccharide/ligand through chelation to the electron donating groups of the ligand/target molecule/saccharide to be coupled.

In another embodiment, the method includes mixing the at least one microsphere with the at least one saccharide at a concentration and size.

In an embodiment, the method includes mixing the least one microsphere with the at least one saccharide, wherein the concentration of the polysaccharide is in range of 0.1 to 10.0 mg/mL, or 0.1 to 8.0 mg/mL or 0.1 to 6.0 mg/mL or 0.1 to 4.0 mg/mL.

In an embodiment, the method includes mixing the least one microsphere with the at least one saccharide, wherein the polysaccharide has a size in range of 0.01 kDa to 3000 kDa.

In a preferred embodiment, the method includes mixing the least one microsphere with the at least one saccharide, the polysaccharide has a size in the range of 0.05 kDa to 2800 kDa, or 0.10 kDa to 2800 kDa, or 0.5 kDa to 2800 kDa, or 1.0 kDa to 2800 kDa, or 2.0 kDa to 2800 kDa, or 5.0 kDa to 2800 kDa, or 10.0 kDa to 2800 kDa, or 15.0 kDa to 2800 kDa, or 20.0 kDa to 2800 kDa, or 25.0 kDa to 2800 kDa, or 30.0 kDa to 2800 kDa, or 35.0 kDa to 2800 kDa, or 40.0 kDa to 2800 kDa, or 45.0 kDa to 2800 kDa, or 50.0 kDa to 2800 kDa.

In another embodiment, the method includes mixing the at least one microsphere with the at least one saccharide in a buffer.

In another embodiment, the method includes mixing the at least one microsphere with the at least one saccharide at a pH in range of 3 to 9.

pH is one of the conjugation condition and pH of 4.5 can work for specific conditions related to saccharide-microsphere couples of Streptococcus pneumoniae Serotype Polysaccharides 2, 6A, 8, 23F, and the like.

In another embodiment, the method includes mixing the at least one microsphere and the at least one saccharide with a chemical composition.

In another embodiment, the step of mixing further includes incubation. In a more preferred embodiment, the at least one microsphere is activated using the bead reagent for 60 mins.

### Mixing: Coupling Ratio

In an embodiment, the microspheres and the saccharides are mixed with each other in a specific coupling ratio.

The specific ratios and assay conditions associated with the saccharide coupled microspheres is provided below.

The coupling ratio is dependent on the requirement of saccharide concentration and blank microsphere/beads per 100 μL. The ratio is calculated as follows:

Coupling ratio=No. of microspheres (Beads) (00 μl)/Concentration of Saccharides (PS) required for coupling (100 μl).

The significance of maximum ratio indicates requirement of less concentration of saccharide for coupling. The calculated broadest coupling ratio for bacterial saccharides is in range of 50 to 12500.

In an embodiment, the mixing is performed with the coupling ratio of the microsphere to the saccharide is in range of 50 to 12500.

In an embodiment, the mixing is performed with the coupling ratio of the microsphere to the pneumococcal saccharides is in range of 50 to 500. In a preferred embodiment, the mixing is performed with the coupling ratio of the microsphere to the pneumococcal saccharides is in range of 50 to 250.

In an embodiment, the mixing is performed with the coupling ratio of the microsphere to the Hib saccharide is in range of 5000 to 12500. In a preferred embodiment, the mixing is performed with the coupling ratio of the microsphere to the Hib saccharide is in range of 8000 to 12500

In an embodiment, the mixing is performed with the coupling ratio of the microsphere to the Neisseria meningitis saccharide is in range of 50 to 12500.

### Mixing: Incubation:

In an embodiment, the mixing further includes incubation.

In a preferred embodiment, the step of mixing further includes incubation at a temperature of 20° C. to 40° C.

In a preferred embodiment, the step of mixing further includes incubation at a temperature of 23° C. to 39° C.

In another embodiment, the present invention is directed to the method of coupling saccharides to microspheres, wherein the mixing is done for incubation temperature in range of 23° C. to 39° C.

In another embodiment, the present invention is directed to the method of coupling saccharides to microspheres, wherein the mixing is done for incubation time of 30 mins to 180 mins.

In an embodiment, the step of mixing further includes incubation time in range of 30 mins to 180 mins. In a preferred embodiment, the incubation time is in range of 30 mins to 150 mins, or in range of 30 mins to 120 mins, or in range of 30 mins to 90 mins. In another preferred embodiment, the incubation time is in 30 mins to 70 mins.

In an embodiment, the method includes mixing the at least one microsphere with the at least one saccharide with the buffer and at the pH in range of 3.0 to 9.0. In an embodiment, the pH of mixing the saccharide with the microsphere is same as the dilution pH of the saccharide.

In an embodiment, the method includes mixing the at least one microsphere with the at least one saccharide with the buffer, at the pH in range of 3 to 9, and incubation.

In an embodiment, the method includes mixing the at least one microsphere with the at least one saccharide with the buffer, at the pH in range of 3 to 9, and incubation at a temperature of 23° C. to 39° C.

In an embodiment, the method includes mixing the at least one microsphere with the at least one saccharide with the buffer, at the pH in range of 3 to 9, and incubation at a temperature of 23° C. to 39° C. for 30 mins to 180 mins.

In a preferred embodiment, method includes mixing the saccharide and microsphere to form saccharide coupled microsphere wherein the saccharide is provided at concentration of 0.1 mg/mL to 10 mg/mL, at a size in range of 0.01 kDa to 3000 kDa, at a pH in range of 3.0 to 9.0, in the buffer for incubation time of 30 mins to 180 mins and at incubation temperature in range of 23° C. to 39° C. to allow development of sites which allow electrostatic attachment of saccharide on to activated beads/microsphere.

### Chemical Composition

In another embodiment, the method includes mixing the at least one microsphere and the at least one saccharide having a chemical composition.

The chemical composition includes at least one component from nitrogen, phosphorus, uronic acids, o-acetyl, methyl-pentoses, hexosamines, or a combination thereof. In a preferred embodiment, the method includes mixing the at least one microsphere and the at least one saccharide having the chemical composition, wherein the chemical composition includes nitrogen and phosphorus.

In another preferred embodiment, the method includes mixing the at least one microsphere and the at least one saccharide having the chemical composition, wherein the chemical composition includes nitrogen content in range of 0.01 mg/ml to 10 mg/ml. The nitrogen content is present as 0-6(%).

In another preferred embodiment, the method includes mixing the at least one microsphere and the at least one saccharide having the chemical composition, wherein the chemical composition includes phosphorus in range of 0.01 mg/ml to 10 mg/ml. The phosphorus is present in form of 0-7(%).

In another preferred embodiment, the method includes mixing the at least one microsphere and the at least one saccharide having the chemical composition, wherein the chemical composition includes nitrogen and phosphorus in the ratio of 1:100 to 100:1.

In another preferred embodiment, the method includes mixing the at least one microsphere and the at least one saccharide having the chemical composition, wherein the chemical composition includes nitrogen, phosphorus and at least one component selected from methyl pentose, hexosamines, uronic acids, O-acetyl.

In another preferred embodiment, the method includes mixing the at least one microsphere and the at least one saccharide having the chemical composition, wherein the chemical composition includes nitrogen, phosphorus and methyl pentose.

In another preferred embodiment, the method includes mixing the at least one microsphere and the at least one saccharide having the chemical composition, wherein the chemical composition includes nitrogen, phosphorus and hexosamines.

In another preferred embodiment, the method includes mixing the at least one microsphere and the at least one saccharide having the chemical composition, wherein the chemical composition includes nitrogen, phosphorus and O-acetyl.

In another preferred embodiment, the method includes mixing the at least one microsphere and the at least one saccharide having the chemical composition, wherein the chemical composition includes nitrogen, phosphorus and uronic acid.

In an embodiment, the method of coupling saccharides to microspheres, the method comprising:
a. providing the microsphere;
b. providing the saccharide;
c. diluting the saccharide with the buffer at pH in range of 3.0 to 9.0; wherein and the saccharide is in range of 0.1 mg/ml to 50.0 mg/ml
d. mixing the microsphere with the saccharide to form the saccharide coupled microsphere;
wherein the saccharide has a size in the range of 50.0 kDa to 3000 kDa; and
wherein the mixing further includes incubation at incubation temperature in range of 20° C. to 40° C. for incubation time in range of 30 mins to 180 mins.

In another embodiment, the method of coupling saccharides to microspheres, the method comprising:
a. providing the microsphere;
b. providing the saccharide;
c. diluting the saccharide with the PBST buffer at pH in range of 3.0 to 6.0; wherein and the saccharide is in range of 0.1 mg/ml to 10.0 mg/ml
d. mixing the microsphere with the saccharide to form the saccharide coupled microsphere;
wherein the saccharide has a size in the range of 50.0 kDa to 3000 kDa; and
wherein the mixing further includes incubation at incubation temperature in range of 23° C. to 39° C. for incubation time in range of 60 to 120 mins.

In another embodiment, the method of coupling saccharides to microspheres, the method comprising:
a. providing the microsphere;
b. providing the saccharide;
c. diluting the saccharide with the PBST buffer at pH in range of 3.0 to 6.0; wherein and the saccharide is in range of 0.1 mg/ml to 10.0 mg/ml
d. mixing the microsphere with the saccharide to form the saccharide coupled microsphere;
wherein the saccharide is a size reduced saccharide and has a size in the range of 50.0 kDa to 3000 kDa; and
wherein the mixing further includes incubation at incubation temperature in range of 23° C. to 39° C. for incubation time in range of 60 to 120 mins.

In an embodiment, the method of coupling the saccharide to the microsphere, the method comprising:
a. providing the microsphere;
b. providing the saccharide;
c. diluting the saccharide with the buffer at pH in range of 3.0 to 9.0;
d. mixing the microsphere with the saccharide to form the saccharide coupled microsphere;
wherein the mixing further includes incubation at temperature in range of 20° C. to 40° C. for incubation time in range of 30 mins to 180 mins,
wherein Streptococcus pneumoniae polysaccharide is selected from serotypes 1, 2, 3, 4, 5, 6, 6A, 6B, 6C, 6D, 7, 7B, 7C, 7F, 8, 9, 9A, 9L, 9N, 9V, 10, 10A, 10B, 10C, 10F, 10X, 11, 11A, 11B, 11C, 11F, 12, 12A, 12B, 12F, 13, 14, 15, 15A, 15B, 15BC, 15C, 15F, 16, 16F, 17, 17A, 17F, 18, 18A, 18B, 18C, 18F, 19, 19A, 19B, 19F, 20, 20A, 20B, 20F, 21, 22, 22A, 22F, 23, 23A, 23B, 23F, 24, 24F, 25, 25F, 26, 27, 28, 28A, 28F, 29, 30, 31, 32, 33, 33A, 33B, 33C, 33D, 33F, 34, 35, 35A, 35B, 35C, 35D, 35F, 36, 37, 38, 39, 40, 41, 42, 43, 44, 45, 46, 47, and/or 48, and
wherein the Streptococcus pneumoniae polysaccharide has a molecular size in range of 50 kDa to 3000 kDa, is diluted with PBST buffer at a pH in range of 3.0 to 9.0 and has concentration of 0.05 mg/mL to 50.0 mg/mL,
wherein the Streptococcus pneumoniae polysaccharide is mixed with the microsphere and incubated at temperature of 23° C. to 39° C., for incubation time of 60 mins to 120 mins, wherein
the mixing is performed with the coupling ratio of the microsphere to the saccharide is in range of 50 to 500, and/or
the microsphere is activated with a bead reagent before mixing with the saccharide, wherein the bead reagent includes metal ions,
optionally, the saccharide coupled microsphere is formed by non-covalent electrostatic coupling, and/or
optionally, the saccharide remains intact, retains epitope confirmation and the saccharide coupled microsphere is stable.

In another embodiment, the method of coupling the saccharide to the microsphere, the method comprising:
a. providing the microsphere;
b. providing the saccharide;
c. diluting the saccharide with the buffer at pH in range of 3.0 to 9.0;
d. mixing the microsphere with the saccharide to form the saccharide coupled microsphere;
wherein the mixing further includes incubation at temperature in range of 20° C. to 40° C. for incubation time in range of 30 mins to 180 mins,
wherein the saccharide is Haemophilus influenzae type b bacteria (Hib) polysaccharide, wherein the Haemophilus influenzae type b bacteria (Hib) polysaccharide has a molecular size in range of 0.1 kDa to 5 kDa, is diluted with PBST buffer at a pH in range of 4 to 6 and has concentration of 1.0 μg/mL to 50.0 μg/mL,
wherein the Haemophilus influenzae type b bacteria (Hib) polysaccharide is mixed with the microsphere and incubated at temperature of 20° C. to 30° C., for incubation time of 60 mins to 120 mins,
wherein
the mixing is performed with the coupling ratio of the microsphere to the saccharide is in range of 5000 to 12500, and/or
the microsphere is activated with a bead reagent before mixing with the saccharide, wherein the bead reagent includes metal ions, and/or
optionally, the saccharide coupled microsphere is formed by non-covalent electrostatic coupling, and/or
optionally, the saccharide remains intact, retains epitope confirmation and the saccharide coupled microsphere is stable.

In another embodiment, the method of coupling the saccharide to the microsphere, the method comprising:
a. providing the microsphere;
b. providing the saccharide;
c. diluting the saccharide with the buffer at pH in range of 3.0 to 9.0;
d. mixing the microsphere with the saccharide to form the saccharide coupled microsphere;
wherein the further mixing includes incubation at temperature in range of 20° C. to 40° C. for incubation time in range of 30 mins to 180 mins,
wherein the polysaccharide is Neisseria meningitidis polysaccharide serotypes selected from meningococcal serotypes A (type I and III), B (type II), B6, B16, C (type II-alpha), D (type IV), Z′/E, E29, H, I, K, K454, L, M, W135, X, Y, Z,
wherein the Neisseria meningitidis polysaccharide has a molecular size in range of 75 kDa to 3000 kDa, is diluted with PBST buffer at a pH in range of 4 to 7 and has concentration of 0.01 mg/mL to 10.0 mg/mL,
wherein the Neisseria meningitidis polysaccharide is mixed with the microsphere and incubated at temperature of 20 to 39° C., for incubation time of 60 mins to 120 mins,
wherein
the mixing is performed with the coupling ratio of the microsphere to the saccharide is in range of 50 to 12500, and/or
the microsphere is activated with a bead reagent before mixing with the saccharide, wherein the bead reagent includes metal ions,
optionally, the saccharide coupled microsphere is formed by non-covalent electrostatic coupling, and/or
optionally, the saccharide remains intact, retains epitope confirmation and the saccharide coupled microsphere is stable.

### Saccharide Coupled Microsphere

In another aspect, the present invention is directed to the saccharide coupled microsphere obtained by the method as disclosed in herein.

In an embodiment, the saccharide coupled microsphere is obtained by the method as disclosed herein, wherein the saccharide coupled microsphere is associated with optimal/percentage range of antibody activity/etc value corresponding to optimal Mean Fluorescence Intensity/MFI values. MFI corresponds to average fluorescence intensity of microspheres after the microsphere have been stained with a fluorescently labelled antibody. Fluorescently labelled microspheres are coupled with the saccharides that act as antigens. When an antibody binds to a microsphere, such binding increases fluorescence intensity of the saccharide coupled microsphere. MFI values is thus obtained for the binding between the microspheres/beads (fluorescent microsphere/beads coupled with the saccharides) and the antibodies as well as the MFI value obtained determines strength of coupling of the saccharide and the microsphere.

In an embodiment of the present disclosure, the saccharide coupled microsphere has Mean Florescence intensity/MFI value in the range of 200 to 20000.

Optimal MFI values are calculated with blank MF less than 100. Optimal MFI value is associated with MFI of 1^{st} standard being 2 to 5 times more than blank, preferably 3 times more than blank. Bead control MFI value is more than at least 5 to 20% of last standard, preferably at least by 10% of last standard. In antigen content assay, for first standard optimal MFI range 200-500 and eight standard optimal MFI range is from 4000 to 20,000.

Optimum antibody dilution range is from 1:5000 to 1:50,000 and optimal antibody concentration ranges from 20 ng to 200 ng required for 4000 beads per well per 50 μL solution.

For 4000 beads per 50 μL of solution, 0.5% to 5% of antibody activity is required for optimal MFI value.

In antigen content assay, for first standard optimal MFI value ranges from 200 to 500 and eight standard optimal MFI ranges from 4000 to 20,000.

The saccharide coupled microsphere obtained by disclosed method is associated with preservation of epitopes even after coupling. Preservation of the epitopes and other parameters of the saccharide coupled microsphere are determined by back fit reference to standards, duplicate % CV, assay blank, and percentage gradations between the MFIs.

The saccharide coupled microsphere obtained by the method as disclosed herein is associated with improved MFI value. The saccharide coupled microsphere in comparison to beads obtained by conventional methods (for e.g. amine coupling) is associated with better/improved MFI's data. The improved MFI data indicates that the saccharide coupled microsphere have higher sensitivities and higher dynamic range.

In an embodiment of the present disclosure, the saccharide coupled microsphere is a Streptococcus pneumoniae saccharide coupled microsphere.

In an embodiment of the present disclosure, the saccharide coupled microsphere is a Haemophilus influenzae type b bacteria (Hib) saccharide coupled microsphere. In an embodiment of the present disclosure, the saccharide coupled microsphere is a Neisseria meningitidis saccharide coupled microsphere.

The saccharide coupled microsphere obtained by the method as disclosed herein is associated with improved stability of beads at different temperature conditions, improved specificity of assay using inhibition experiments, consistent linearity, accuracy, precision, LOQ parameters.

During qualification stage the system suitability parameters are monitored such as Back fit of the reference standards, duplicate % CV, assay blank and % gradation between the MFI's. When all the parameters are complying to the corresponding specification indicate that the epitopes are intact, as the assessment method is based on the ‘antigen-antibody reaction’. If epitopes are damaged antibody binding to antigen will be inappropriate and impact on the standard curve formation and duplicate % CV of the assay.

### Streptococcal Polysaccharides & Coupling

In another embodiment, the present invention is directed to the method of coupling saccharides to microspheres, wherein the saccharide is Streptococcus polysaccharide.

In another embodiment, the present invention is directed to the method of coupling saccharides to microspheres, wherein the saccharide from Streptococcus pneumoniae including Group A Streptococcus, Group B Streptococcus.

In another embodiment, the present invention is directed to the method of coupling saccharides to microspheres, wherein the saccharide is Streptococcus pneumoniae polysaccharide.

In another embodiment, the present invention is directed to the method of coupling polysaccharides to microspheres, wherein the polysaccharide is Streptococcus pneumoniae polysaccharide selected from serotypes 1, 2, 3, 4, 5, 6, 6A, 6B, 6C, 6D, 7, 7B, 7C, 7F, 8, 9, 9A, 9L, 9N, 9V, 10, 10A, 10B, 10C, 10F, 10X, 11, 11A, 11B, 11C, 11F, 12, 12A, 12B, 12F, 13, 14, 15, 15A, 15B, 15BC, 15C, 15F, 16, 16F, 17, 17A, 17F, 18, 18A, 18B, 18C, 18F, 19, 19A, 19B, 19F, 20, 20A, 20B, 20F, 21, 22, 22A, 22F, 23, 23A, 23B, 23F, 24, 25, 25F, 26, 27, 28, 28A, 28F, 29, 30, 31, 32, 33, 33A, 33B, 33C, 33D, 33F, 34, 35, 35A, 35B, 35C, 35D, 35F, 36, 37, 38, 39, 40, 41, 42, 43, 44, 45, 46, 47, and/or 48.

In another embodiment, the present invention is directed to the method of coupling polysaccharides to microspheres, wherein the polysaccharide is Streptococcus pneumoniae polysaccharide selected from serotypes 1, 2, 4, 5, 6A, 6B, 7F, 8, 9V, 10A, 11A, 12F, 14, 15B, 18C, 19A, 19F, 22F, 23F, 24F and/or 33F.

In another embodiment, the present invention is directed to the method of coupling polysaccharides/saccharides to microspheres, wherein the Streptococcus pneumoniae polysaccharide has molecular size in the range of 50 kDa to 3000 kDa, preferably in the range of 50 kDa to 1500 kDa.

In an embodiment, the polysaccharide/saccharide used as is native polysaccharide/saccharide.

In a preferred embodiment, the saccharide of serotype 2, 6A, 8 are of native size.

In an alternate embodiment, the saccharide is reduced in size. In a preferred embodiment, the saccharide of serotype 2, 6A, 8 or all are reduced in size.

In a more preferred embodiment, the saccharide is a modified saccharide. The Streptococcus pneumoniae serotype 2 modified saccharide has size 80 kDa and after coupling was observed to be associated with the lesser MFI value compared with the native saccharide (Size 1011 kDa) which gives a good increase in the MFI value.

In an embodiment, the present invention is directed to the method of coupling saccharides to microspheres, wherein concentration of saccharide is in range of 0.05 mg/mL to 50.0 mg/mL.

In an embodiment, the present invention is directed to the method of coupling saccharides to microspheres, wherein concentration of saccharide is in range of 0.05 mg/mL to 10.0 mg/mL.

In a preferred embodiment, the present invention is directed to the method of coupling saccharides to microspheres, wherein concentration of saccharide is in range of 0.05 mg/mL to 8.0 mg/mL, or in range of 0.05 mg/mL to 6.0 mg/mL, or in range of 0.05 mg/mL to 5.0 mg/mL, or in range of 0.05 mg/mL to 4.0 mg/mL.

In another preferred embodiment, the present invention is directed to the method of coupling saccharides to microspheres, wherein concentration of saccharide is in range of 0.1 mg/mL to 4.0 mg/mL, or in range of 0.1 mg/ml to 3.0 mg/ml, or in range of 0.1 mg/ml to 2.5 mg/ml.

In an embodiment, the method includes diluting the at least one microsphere with the at least one Streptococcus pneumoniae polysaccharide with the buffer and at the pH in range of 3.0 to 9.0.

In an embodiment, the method includes diluting the at least one microsphere with the at least one Streptococcus pneumoniae polysaccharide with the buffer and at the pH in range of 4.0 to 7.0.

In an embodiment, the method includes diluting the at least one microsphere with the at least one Streptococcus pneumoniae polysaccharide with the buffer, at the pH in range of 3.0 to 9.0, and then mixing.

In an embodiment, the method includes mixing the at least one microsphere with the at least one Streptococcus pneumoniae polysaccharide with incubation at a temperature of 23° C. to 39° C.

In an embodiment, the method includes mixing the at least one microsphere with the at least one Streptococcus pneumoniae polysaccharide with incubation at a temperature of 23° C. to 39° C. for incubation time in range of 30 mins to 180 mins.

In an embodiment, the method includes mixing the at least one microsphere with the at least one Streptococcus pneumoniae polysaccharide with incubation at a temperature of 23° C. to 39° C. for incubation time in range of 60 mins to 180 mins.

In an embodiment, the method includes mixing the at least one microsphere with the at least one Streptococcus pneumoniae polysaccharide with incubation at a temperature of 25° C. to 37° C. for incubation time in range of 60 mins to 180 mins.

In an embodiment, the method includes mixing the at least one microsphere with the at least one Streptococcus pneumoniae polysaccharide with incubation at a temperature of 25° C. to 37° C. for incubation time in range of 60 mins to 120 mins.

In a preferred embodiment, the stock of native Streptococcus pneumoniae polysaccharide saccharide is diluted in a buffer prior to coupling.

In another embodiment, the saccharide is Streptococcus pneumoniae polysaccharide/saccharide selected from a group of polysaccharides associated with Streptococcus pneumoniae/pneumococcal strains associated with global Streptococcus pneumoniae/pneumococcal sequence cluster (GPSC) selected from GPSC 1 to 131.

In another embodiment, a composition comprising Streptococcus pneumoniae polysaccharide/saccharide coupled microsphere is obtained by the method as disclosed herein.

The Streptococcus pneumoniae polysaccharide/saccharide coupled microsphere obtained by the method as disclosed herein is associated with improved MFI value.

The Streptococcus pneumoniae polysaccharide/saccharide coupled microsphere in comparison to beads obtained by conventional methods (for e.g. amine coupling) is associated with better/improved MFI's data. The improved MFI data indicates that the Streptococcus pneumoniae polysaccharide saccharide coupled microsphere has higher sensitivity and higher dynamic range.

In an embodiment, the Streptococcus pneumoniae polysaccharide/saccharide coupled microsphere for serotypes 7F, 8, 10A and 24F are associated with improved higher MFI's than the beads obtained by the conventional methods indicating higher sensitivity.

### Haemophilus Saccharide & Coupling

In another embodiment, in the method as disclosed herein, the saccharide is associated Haemophilus bacteria, Haemophilus influenzae bacteria, or Haemophilus influenzae type b bacteria (Hib).

In another embodiment, in the method as disclosed herein, the saccharide is Haemophilus influenzae type a, type b, type c, type d, type e, or type f bacteria polysaccharide.

In another embodiment, in the method as disclosed herein, the saccharide is Haemophilus influenzae type b bacteria (Hib) polysaccharide.

In another embodiment, in the method as disclosed herein, the saccharide is Haemophilus influenzae type b bacteria (Hib) saccharide selected from a group of saccharides associated with type I and type II Hib strains.

In another embodiment, in the method as disclosed herein, the Haemophilus influenzae type b bacteria (Hib) saccharide is associated with a molecular size of 0.05 kDa to 3000 kDa.

In another embodiment, in the method as disclosed herein, the Haemophilus influenzae type b bacteria (Hib) saccharide is associated with a molecular size of 0.05 kDa to 5 kDa.

In another embodiment, in the method as disclosed herein, the Haemophilus influenzae type b bacteria (Hib) saccharide is associated with a molecular size of 0.1 kDa to 1 kDa.

In another embodiment, in the method as disclosed herein, the Haemophilus influenzae type b bacteria (Hib) saccharide is associated with a molecular size of 0.3 kDa for more than 50% of the saccharides.

In an embodiment, the method includes diluting the at least one microsphere with the at least one Hib polysaccharide with the PBST buffer at the pH in range of 3.0 to 9.0.

In an embodiment, the method includes diluting the at least one microsphere with the at least one Hib polysaccharide with the PBST buffer at the pH in range of 4.0 to 6.0.

In another embodiment, in the method as disclosed herein, concentration of Haemophilus influenzae type b bacteria (Hib) saccharide is in range of 1.0 μg/mL to 50.0 μg/mL.

In another embodiment, in the method as disclosed herein, concentration of Haemophilus influenzae type b bacteria (Hib) saccharide is in range of 5 μg/mL to 15 μg/mL.

In another embodiment, in the method as disclosed herein, the saccharide is Haemophilus influenzae type b bacteria (Hib) saccharide selected from a group of saccharide associated with Haemophilus influenzae type b bacteria (Hib) strains associated variation of hcsA gene.

In an embodiment, the method includes mixing the at least one microsphere at incubation temperature of 20° C. to 30° C.

In an embodiment, the method includes mixing the at least one microsphere at incubation temperature of 20° C. to 30° C. for 30 mins to 180 mins.

In an embodiment, the method includes mixing the at least one microsphere at incubation temperature of 25° C. for 60 mins to 120 mins.

In another embodiment, the composition comprising Haemophilus influenzae type b bacteria (Hib) saccharide coupled microsphere is obtained by the method as disclosed herein.

### Meningococcal Saccharides & Coupling

In another embodiment, in the method as disclosed herein, the saccharide is associated with Neisseria meningitidis/meningococcus.

In another embodiment, in the method as disclosed herein, the saccharide is Neisseria meningitidis polysaccharide.

In another embodiment, in the method as disclosed herein, the saccharide is Neisseria meningitidis saccharide selected from meningococcal serotypes A (type I and III), B (type II), B6, B16, C (type II-alpha), D (type IV), Z′/E, E29, H, I, K, K454, L, M, W135, X, Y, Z.

In another embodiment, in the method as disclosed herein, the saccharide is Neisseria meningitidis saccharide selected from meningococcal serotypes A, B, C, W, X, and Y.

In another embodiment, in the method as disclosed herein, the Neisseria meningitidis saccharide is associated with a size of 75 kDa to 3000 kDa.

In another embodiment, in the method as disclosed herein, concentration of Neisseria meningitidis saccharide is in range of 0.01 mg/mL to 10.0 mg/mL.

In an embodiment, the method includes mixing the at least one microsphere with the at least one Neisseria meningitidis polysaccharide with the buffer and at the pH in range of 3.0 to 9.0.

In an embodiment, the method includes mixing the at least one microsphere with the at least one Neisseria meningitidis polysaccharide with the buffer, at the pH in range of 3.0 to 9.0, and incubation.

In an embodiment, the method includes mixing the at least one microsphere with the at least one Neisseria meningitidis polysaccharide with the buffer, at the pH in range of 3.0 to 9.0, and incubation at a temperature in range of 20° C. to 40° C., preferably in range of 23° C. to 39° C.

In an embodiment, the method includes mixing the at least one microsphere with the at least one Neisseria meningitidis polysaccharide with the buffer, at the pH in range of 3.0 to 9.0, and incubation at a temperature of 23° C. to 39° C. for 30 mins to 180 mins.

In another embodiment, in the method as disclosed herein, the saccharide is Neisseria meningitidis saccharide selected from a meningococcal strain associated with variation in IpxL1, fHbp, and tps genes.

In an embodiment, the composition comprising Neisseria meningitidis saccharide coupled microsphere is obtained by the method as disclosed herein.

### Another Aspect: Method of Evaluating Immunogenicity

In another aspect, the present invention is directed to the saccharide coupled microspheres as and when used to determine antibody titre of an immunogenic composition/a vaccine.

In an embodiment, the present invention is directed to a method of evaluating immunogenicity of immunogenic composition, the method comprising;
a. providing a test sample corresponding to the saccharide in the immunogenic composition;
b. providing the saccharide coupled microsphere corresponding to the saccharide in the immunogenic composition; and
c. evaluating the immunogenicity of the immunogenic composition by antigen content determination, or estimating antibody concentration (IgG), or identity assay, free polysaccharide estimation using the test sample and the saccharide coupled microsphere.

### Antigen Content Determination

In another aspect, the present invention is directed to an antigen content determination method using the saccharide coupled microsphere obtained by the method as disclosed herein.

In an embodiment, the antigen content determination method using the saccharide coupled microsphere comprises:
i. desorbing the test sample and the standard,
ii. preparing the percent adsorption sample,
iii. preparing mixture of antisera/Monoclonal antibody (mAb) for at least one serotype specific saccharide,
iv. diluting standard, test sample and percent adsorption sample,
v. transferring the standard, the sample, and the bead control into respective wells containing mixture of sera/Mab, incubate the dilution plate,
vi. providing the saccharide coupled microsphere/bead for at least one serotype specific polysaccharide,
vii. preparing assay plate by adding the coupled beads in each well of the plate, followed by adding the standard and the sample from dilution plate to wells in the assay plate having beads as well as bead blanks, incubating and washing, Washing and dilution is performed using reagent D. (i.e. Luminex Assay Buffer, LAB),
viii. preparing anti rabbit phycoerythrin and anti-mice phycoerythrin and adding in each well,
ix. adding reagent D to the plate, and
x. reading the plate on protein suspension array system.

In an embodiment, the desorption is performed with a desorption buffer. The desorption buffer includes sodium citrate and/or 1 M Sodium Hydroxide (˜140 μL for 2 mL).

In an embodiment, reagent D is interchangeably referred to as Luminex Assay Buffer.

Reagent D includes bovine serum albumin (BSA), Tween 20 PBS, reagent C, reagent B. solution is maintained at pH 7+/−0.2 with 1 N NaOH or 1 M Citric acid and filtered through 0.22μ filter.

In a preferred embodiment, the antigen content determination method using the saccharide coupled microsphere comprises:
i. providing a standard solution of polysaccharides,
ii. providing a test sample,
iii. desorbing the test sample and the standard,
iv. preparing the percent adsorption sample,
v. preparing mixture of antisera/Monoclonal antibody (Mab) for at least one serotype specific saccharide,
vi. dilute test sample and percent adsorption sample,
vii. transfer the standard, the sample, and the bead control into respective wells containing mixture of sera/mAb, incubate the dilution plate,
viii. providing the saccharide coupled microsphere/bead for at least one serotype specific polysaccharide,
ix. preparing assay plate by adding the beads in each well of the plate, followed by adding the standard and the sample from dilution plate to wells in the assay plate having beads as well as bead blanks, incubating and washing,
x. preparing anti rabbit phycoerythrin and anti-mice phycoerythrin and adding in each well,
xi. incubating the plate,
xii. washing the plate, Washing and dilution is performed using reagent D,
xiii. adding reagents D to the plate, and
xiv. reading the plate on protein suspension array system.

In an embodiment, reagent D is interchangeably referred to has Luminex Assay Buffer.

Reagent D includes bovine serum albumin (BSA), Tween 20 PBS, reagent C, reagent B. solution is maintained at pH 7+/−0.2 with 1 N NaOH or 1 M Citric acid. Filtered through 0.22μ filter.

See more detailed method of antigen content determination assay in the “METHODS” herein.

### Identity Assay

In another aspect, the present invention is directed to an identity assay method using the saccharide coupled microsphere obtained by the method as disclosed herein.

In an embodiment, the identity assay method using the saccharide coupled microsphere comprises:
i. desorbing the test sample and the standard,
ii. preparing mixture of antisera/Monoclonal antibody (Mab) for at least one serotype specific saccharide,
iii. dilute test sample (with Luminex Assay Buffer),
iv. transfer the standard, the sample, and the bead control into respective wells containing mixture of sera/Mab, incubate the dilution plate,
v. providing the saccharide coupled microsphere/bead for at least one serotype specific polysaccharide,
   preparing assay plate by adding the beads in each well of the plate, followed by adding the standard and the sample from dilution plate to wells in the assay plate having beads as well as bead blanks, incubating and washing
   Washing and dilution is performed using reagent D.
vi. preparing anti rabbit phycoerythrin and anti-mice phycoerythrin and adding in each well,
vii. adding reagents D to the plate, and
viii. reading the plate on protein suspension array system.

In an embodiment, the desorption is performed with a desorption buffer. The desorption buffer includes sodium citrate.

In an embodiment, reagent D is interchangeably referred to has Luminex Assay Buffer.

Reagent D includes bovine serum albumin (BSA), Tween 20 PBS, reagent C, reagent B. solution is maintained at pH 7+/−0.2 with 1 N NaOH or 1 M Citric acid and filtered through 0.22μ filter)

In a preferred embodiment, the identity assay method using the saccharide coupled microsphere comprises:
i. providing a standard solution of polysaccharides,
ii. providing a test sample,
iii. desorbing the test sample and the standard,
iv. preparing mixture of antisera/Monoclonal antibody (Mab) for at least one serotype specific saccharide,
v. dilute test sample (with Luminex Assay Buffer),
vi. transfer the standard, the sample, and the bead control into respective wells containing mixture of sera/mAb, incubate the dilution plate,
vii. providing the saccharide coupled microsphere/bead for at least one serotype specific polysaccharide,
viii. preparing assay plate by adding the beads in each well of the plate, followed by adding the standard and the sample from dilution plate to wells in the assay plate having beads as well as bead blanks, incubating and washing,
ix. preparing anti rabbit phycoerythrin and anti-mice phycoerythrin and adding in each well,
x. incubating the plate,
xi. washing the plate,
xii. adding reagents D to the plate, and
xiii. reading the plate on protein suspension array system.

See more detailed method of identity assay in the “METHODS” herein.

### Free Saccharide Estimation

In another aspect, the present invention is directed to a free saccharide estimation method using the saccharide coupled microsphere obtained by the method as disclosed herein.

In an embodiment, the free saccharide estimation method using the saccharide coupled microsphere comprises:
i. preparing free saccharides samples,
ii. preparing mixture of antisera/Monoclonal antibody (Mab) for at least one serotype specific saccharide,
iii. dilute test sample (with Luminex Assay Buffer),
iv. transfer the standard, the sample, and the bead control into respective wells containing mixture of sera/mAb, incubate the dilution plate,
v. providing the saccharide coupled microsphere/bead for at least one serotype specific polysaccharide,
vi. preparing assay plate by adding the beads in each well of the plate, followed by adding the standard and the sample from dilution plate to wells in the assay plate having beads as well as bead blanks, incubating and washing,
vii. preparing anti rabbit phycoerythrin and anti-mice phycoerythrin and adding in each well,
viii. adding reagents D to the plate and
ix. reading the plate on protein suspension array system.

In a preferred embodiment, the free saccharide estimation method using the saccharide coupled microsphere comprises:
i. providing a standard solution of polysaccharides,
ii. providing a test sample,
iii. preparing free Saccharides samples,
iv. preparing mixture of antisera/Monoclonal antibody (Mab) for at least one serotype specific saccharide,
v. dilute test sample (with Luminex Assay Buffer),
vi. transfer the standard, the sample, and the bead control into respective wells containing mixture of sera/mAb, incubate the dilution plate,
vii. providing the saccharide coupled microsphere/bead for at least one serotype specific polysaccharide,
viii. preparing assay plate by adding the beads in each well of the plate, followed by adding the standard and the sample from dilution plate to wells in the assay plate having beads as well as bead blanks, incubating and washing,
ix. preparing anti rabbit phycoerythrin and anti-mice phycoerythrin and adding in each well,
x. incubating the plate,
xi. washing the plate,
xii. adding reagent D to the plate and
xiii. reading the plate on protein suspension array system.

See more detailed method of the free saccharide estimation assay method in the “METHODS” herein.

### Estimating Antibody Concentration (IGG) in Sera Sample

In another aspect, the present invention is directed to a method of estimating antibody concentration (IgG) in sera sample, the method using the saccharide coupled microsphere obtained by the method as disclosed herein.

In an embodiment, the method of estimating antibody concentration (IgG) in sera sample using the saccharide coupled microsphere comprises:
i. providing the saccharide coupled microsphere/bead for at least one serotype specific polysaccharide,
ii. preparing assay plate by adding the beads in each well of the plate, followed by adding the standard and the sample from dilution plate to wells in the assay plate having beads as well as bead blanks, incubating and washing,
iii. preparing anti rabbit phycoerythrin and anti-mice phycoerythrin and adding in each well,
iv. adding reagents to the plate and
v. reading the plate on protein suspension array system.

In a preferred embodiment, the method of estimating antibody concentration (IgG) in sera sample using the saccharide coupled microsphere comprises:
i. providing a standard solution of polysaccharides,
ii. providing a test sample,
iii. providing the saccharide coupled microsphere/bead for at least one serotype specific polysaccharide,
iv. preparing assay plate by adding the beads in each well of the plate, followed by adding the standard and the sample from dilution plate to wells in the assay plate having beads as well as bead blanks, incubating and washing,
v. preparing anti rabbit phycoerythrin and anti-mice phycoerythrin and adding in each well,
vi. incubating the plate,
vii. washing the plate,
viii. adding reagents to the plate and
ix. reading the plate on protein suspension array system.

See more detailed method of estimating antibody concentration (IgG) in sera sample using the saccharide coupled microsphere in the “METHODS” herein.

In an embodiment, for the method of estimating antibody concentration (IgG), the sera sample includes a human sera sample.

In a preferred embodiment, for the method of estimating antibody concentration (IgG), the sera sample includes an animal sera sample.

In another preferred embodiment, for the method of estimating antibody concentration (IgG), the sera sample includes a human sera sample and an animal sera sample.

### Another Aspect: Apparatus

In another aspect, the present invention is directed to an apparatus including the saccharide coupled microsphere.

### Another Aspect: Application

In another aspect, the present invention is directed to the saccharide coupled microspheres as and when used to determine antibody titre of an immunogenic composition/a vaccine.

### Methods

### Method of Evaluating Immunogenicity

In an embodiment, the present invention is directed to a method of evaluating immunogenicity of immunogenic composition, the method comprising;
a. providing a test sample corresponding to the saccharide in the immunogenic composition;
b. providing the saccharide coupled microsphere corresponding to the saccharide in the immunogenic composition;
c. evaluating the immunogenicity of the immunogenic composition by antigen content determination, or estimating antibody concentration (IgG), or identity assay, free polysaccharide estimation using the test sample and the saccharide coupled microsphere.

### Antigen Content Determination

The antigen content determination method using the saccharide coupled microsphere comprises:

### Preparation of Standard:

Prepare 21 Valent Reference standard: Mix all the serotypes (except 6B) at 4.4 μg/ml and serotype 6B at 8.8 μg/ml. Prepare the series of standard from S1 to S8 in the first column of 96-well titer plate using reagent D by serial 2-fold dilution as per the following table. Discard 125 μl from the last well after mixing. Final total volume of each well should be 125 μl.

### Preparation of Sample:

Take 2 ml of test sample.

### Desorption of Sample and Standard:

Desorption is done with desorption buffer. Desorption buffer includes sodium citrate.

Add 200 mg sodium citrate per 2 ml of sample and standard, incubate for 2 Hrs at 37° C. on roto-spin for complete desorption.

### Preparation of % Adsorption Sample:

Take 1.0 mL of sample in tube and centrifuged it at 10000 g for 10 minutes and collect supernatant.

### Preparation Mixture of Sera/mAb:

Prepare the 21 Valent mixture of Antisera/Mab using the optimized dilution for each serotype Procedure:

### Preparation Dilution Plate:

Prepare the series of standard from S1 to S8 by serial 2-fold dilution. Appropriately dilute the test sample & percent adsorption sample with LAB to fit in the standard curve.

Transfer 100 μl of the standards, sample and bead control to the respective wells, containing sera/mAb in the same plate.

Incubate the plate in shaking incubator at 37° C. for 1 hour with shaking at 150 rpm.

### Preparation Mixture of Beads:

Prepared the 21 valent mixture of beads in Luminex Assay Buffer.

### Preparation of Assay Plate:

Add 4000 beads per well per 50 μl in each required wells of filter plate.

Add 50 μl of incubated standards and samples from dilution plate in duplicates on filter plate containing beads with bead control and blank Incubate the plate in shaking incubator at 37° C. for 1 hour with shaking at 150 rpm. Wash the plate with 100 ul of LAB. This step should be performed 3 times.

### Preparation of Phycoerythrin (PE):

Prepare the mixture of required volume of anti-Rabbit-phycoerythrin and anti-mice-phycoerythrin.

Add 50 μl in each well. Incubate the plate in shaking incubator at 37° C. for 30 minutes with shaking at 150 rpm.

Wash the plate with 100 μl of reagent D, 3 times.

Add 100 ul of reagent D and read the plate on Protein Suspension Array System (Bio-Plex 200).

Adding reagents D (Luminex Assay Buffer: Dissolve 2.0 gm bovine serum albumin (BSA), 2.5 mL reagent C and 1 mL Tween 20 in 800 mL of 1×PBS. Make up the volume to 1000 mL with reagent B. Adjust the pH of the solution to 7.2±0.1 with 1 N NaOH or 1 M Citric acid. Filter through 0.22μ filter.

### Identity Assay

Preparation of standard:

Prepare 21 Valent Reference standard: Mix all the serotypes at 4.4 μg/ml except 6B and serotype 6B at 8.8 μg/ml

### Preparation of Sample:

Take 2 ml of test sample.

### Desorption of Sample and Standard:

Desorption is done with desorption buffer. Desorption buffer includes sodium citrate and or 1 M sodium hydroxide.

Add 200 mg sodium citrate per 2 ml of sample and standard, incubate for 2 Hrs at 37° C. on roto-spin for complete desorption.

Preparation Mixture of sera/mAb:

Prepare the 21 Valent mixture of Antisera/Mab using the optimized dilution for each serotype.

### Procedure:

### Preparation Dilution Plate:

Prepare the series of standard from S1 to S8 by serial 2-fold dilution. Appropriately dilute the test sample to fit in the standard curve.

Transfer 100 μl of the standards, sample and bead control to the respective wells, containing sera/mAb in the same plate.

Incubate the plate in shaking incubator at 37° C. for 1 hour with shaking at 150 rpm.

### Preparation Mixture of Beads:

Prepared the 21 valent mixture of beads in Luminex Assay Buffer

### Preparation of Assay Plate:

Add 4000 beads per well per 50 μl in each required well of filter plate.

Add 50 μl of incubated standards and samples from dilution plate in duplicates on filter plate containing beads with bead control and blank Incubate the plate in shaking incubator at 37° C. for 1 hour with shaking at 150 rpm.

Wash the plate with 100 ul of LAB. This step should be performed 3 times.

### Preparation of Phycoerythrin (PE):

Prepare the mixture of required volume of anti-Rabbit-phycoerythrin and anti-mice-phycoerythrin

Add 50 μl in each well. Incubate the plate in shaking incubator at 37° C. for 30 minutes with shaking at 150 rpm.

Wash the plate with 100 μl of reagent D, 3 times.

Add 100 ul of reagent D and read the plate on Protein Suspension Array System (Bio-Plex 200).

Adding reagents D (Luminex Assay Buffer: Dissolve 2.0 gm bovine serum albumin (BSA), 2.5 mL reagent C and 1 mL Tween 20 in 800 mL of 1×PBS. Make up the volume to 1000 mL with reagent B. Adjust the pH of the solution to 7.2±0.1 with 1 N NaOH or 1 M Citric acid. Filter through 0.22μ filter).

### Free Saccharide Estimation in Drug Product

### Preparation of Standard:

Prepared 21 Valent Reference standard: Mix all the serotypes at 4.4 μg/ml except 6B and serotype 6B at 8.8 μg/ml

### Preparation of Sample:

Take 2 ml supernatant of test sample by centrifuged at 5000 g for 5 min.

### Preparation of Free PS Sample:

A) Method-A: Preparation for free saccharide content using deoxycholate and hydrochloric acid. From the final sample after DOC precipitation, take the appropriate aliquot so as to fit in the standard curve range.
B) Method-B: Preparation of free saccharide content using the three different mAb coupled with Beads are incubated with the test solution for 1 Hr at 37° C. at 150 rpm. Further centrifuge the sample at 5000 g for 5 min and collect the supernatant.
   Preparation Mixture of sera/mAb:

Prepare the 21 Valent mixture of Antisera/Mab using the optimized dilution for each serotype.

### Procedure:

### Preparation Dilution Plate:

Prepare the series of standard from S1 to S8 by serial 2-fold dilution. Appropriately dilute the test sample.

Transfer 100 μl of the standards, sample and bead control to the respective wells, containing sera/mAb in the same plate.

Incubate the plate in shaking incubator at 37° C. for 1 hour with shaking at 150 rpm.

### Preparation Mixture of Beads:

Prepared the 21 valent mixture of beads in Luminex Assay Buffer

### Preparation of Assay Plate:

Add 4000 beads per well per 50 μl in each required well of filter plate.

Add 50 μl of incubated standards and samples from dilution plate in duplicates on filter plate containing beads with bead control and blank

Incubate the plate in shaking incubator at 37° C. for 1 hour with shaking at 150 rpm.

Wash the plate with 100 ul of LAB. This step should be performed 3 times.

### Preparation of PE:

Prepare the mixture of required volume of anti-Rabbit-phycoerythrin and anti-mice-phycoerythrin

Add 50 μl in each well. Incubate the plate in shaking incubator at 37° C. for 30 minutes with shaking at 150 rpm.

Wash the plate with 100 μl of reagent D, 3 times.

Add 100 ul of reagent D and read the plate on Protein Suspension Array System (Bio-Plex 200).

Adding reagents D (Luminex Assay Buffer: Dissolve 2.0 gm bovine serum albumin (BSA), 2.5 mL reagent C and 1 mL Tween 20 in 800 mL of 1×PBS. Make up the volume to 1000 mL with reagent B. Adjust the pH of the solution to 7.2±0.1 with 1 N NaOH or 1 M Citric acid. Filter through 0.22μ filter).

### Estimation of Antibody Concentration (IGG) in Clinical (Human) Sera Samples.

### Preparation of Standard:

Prepare the working reference standard by diluting the 007SP reference sera from 1:500 to 1:64000

### Preparation of Sample:

Generally, test sample dilutions scheme as 1:200, 1:400, 1:800, 1:1600 fits well in the standard curve.

### Procedure:

### Preparation Mixture of Beads:

Prepared the 21 valent mixture of beads in Luminex Assay Buffer

### Preparation of Assay Plate:

Prewet the required wells of multiscreen filter plate with 100 μl of reagent D and aspirate the plate using vacuum manifold

Add 4000 beads per well per 50 μl in each required well of filter plate. Aspirate the filter plate using vacuum manifold

Add 50 μl of incubated standards and samples from dilution plate in duplicates on filter plate containing beads with bead control and blank

Incubate the plate in shaking incubator at 37° C. for 1 hour with shaking at 150 rpm.

Wash the plate with 100 ul of LAB. This step should be performed 3 times.

### Preparation of PE:

Prepare the mixture of required volume of anti-Rabbit-phycoerythrin and anti-mice-phycoerythrin

Add 50 μl in each well. Incubate the plate in shaking incubator at 37° C. for 30 minutes with shaking at 150 rpm.

Wash the plate with 100 μl of reagent D, 3 times.

Add 100 ul of reagent D and read the plate on Protein Suspension Array System (Bio-Plex 200).

Adding reagents D (Luminex Assay Buffer: Dissolve 2.0 gm bovine serum albumin (BSA), 2.5 mL reagent C and 1 mL Tween 20 in 800 mL of 1×PBS. Make up the volume to 1000 mL with reagent B. Adjust the pH of the solution to 7.2±0.1 with 1 N NaOH or 1 M Citric acid. Filter through 0.22μ filter).

### Estimation of IGG Titter in Animal Sera Sample

### Preparation of Standard:

Prepare the working reference standard by diluting the 007SP reference sera from 1:500 to 1:64000

### Preparation of Sample:

Generally, test sample dilutions scheme as 1:200, 1:400, 1:800, 1:1600 fits well in the standard curve.

### Procedure:

### Preparation Mixture of Beads:

Prepare the 21 valent mixture of beads in Luminex Assay Buffer

### Preparation of Assay Plate:

Prewet the required wells of multiscreen filter plate with 100 μl of reagent D and aspirate the plate using vacuum manifold.

Add 4000 beads per well per 50 μl in each required well of filter plate. Aspirate the filter plate using vacuum manifold. Add 50 μl of incubated standards and samples from dilution plate in duplicates on filter plate containing beads with bead control and blank.

Incubate the plate in shaking incubator at 37° C. for 1 hour with shaking at 150 rpm.

Wash the plate with 100 ul of LAB. This step should be performed 3 times.

### Preparation of PE:

Prepare the mixture of required volume of anti-Rabbit-phycoerythrin and anti-mice-phycoerythrin Add 50 μl in each well. Incubate the plate in shaking incubator at 37° C. for 30 minutes with shaking at 150 rpm.

Wash the plate with 100 μl of reagent D, 3 times.

Add 100 ul of reagent D and read the plate on Protein Suspension Array System (Bio-Plex 200). Adding reagents D (Luminex Assay Buffer: Dissolve 2.0 gm bovine serum albumin (BSA), 2.5 mL reagent C and 1 mL Tween 20 in 800 mL of 1×PBS. Make up the volume to 1000 mL with reagent B. Adjust the pH of the solution to 7.2±0.1 with 1 N NaOH or 1 M Citric acid. Filter through 0.22μ filter).

The present invention is an alternative method for coupling of saccharides like antigens to microsphere. The proposed method is based on principles of metal coordination chemistry and electrostatic mode of interaction-based coupling of purified bacterial saccharides to microsphere, such coupled microsphere is subsequently used for saccharide-protein related conjugate vaccine. Bio-assays are selected from but not limited to a) antigen content and b) IgG determination-serology-based applications, c) identity assays and d) stability assays (Free saccharide estimation).

The improved method of coupling the microsphere with the saccharides utilizes specific advantageous parameters related to buffer type, pH, incubation time, chemicals used, size of the polysaccharides, serotype of bacterial polysaccharides, as compared to previous conventional covalent coupling assays.

### EMBODIMENTS

The present invention is illustrated in more detail by the following embodiments and combinations of embodiments which result from the corresponding dependency references and links:
I. A method of coupling a saccharide to a microsphere to obtain a saccharide coupled microsphere, the method comprising:
   a. providing the microsphere;
   b. providing the saccharide;
   c. diluting the saccharide with a buffer at pH in range of 3.0 to 9.0; and
   d. mixing the microsphere with the saccharide to form the saccharide coupled microsphere;
wherein the mixing further includes incubation at temperature in range of 20° C. to 40° C. for incubation time in range of 30 mins to 180 mins.
II. The method of coupling a saccharide to a microsphere as disclosed in embodiment I, wherein, the method comprises:
   a. providing the microsphere;
   b. providing the saccharide;
   c. diluting the saccharide with the buffer at pH in range of 3.0 to 9.0; optionally wherein and the saccharide concentration is in range of 1.0 μg/ml to 50.0 mg/ml, preferably in the range of 1.0 μg/ml to 10 mg/ml:
   d. mixing the microsphere with the saccharide to form the saccharide coupled microsphere;
optionally, wherein the saccharide has a size in the range of 0.05 kDa to 3000 kDa, preferable in the range of 0.05 kDa to 1500 kDa; and optionally, wherein the mixing includes incubation at temperature in range of 20° C. to 40° C. for incubation time in range of 30 mins to 180 mins.
III. The method as disclosed in embodiment II, wherein
   optionally, the mixing is performed with the coupling ratio of the microsphere to the saccharide is in range of 50 to 12500, preferably in range of 50 to 250, and/or
   optionally, the metal ions are adsorbed on surface of the microsphere during activation of the microsphere before mixing with the saccharide,
   optionally, the saccharide coupled microsphere is formed by non-covalent electrostatic coupling, and/or
   optionally, the saccharide remains intact, retains epitope confirmation and the saccharide coupled microsphere is stable.
IV. The method as disclosed in any one of embodiments I to II, wherein the buffer includes phosphate buffered saline with tween (PBST) buffer, (4-(2-hydroxyethyl)-1-piperazineethane sulfonic acid (HEPES) buffer, tris-aminomethane (Tris) buffer, 2-(N-morpholino)ethane sulfonic acid (MES) buffer, 3-(N-morpholino) propane sulfonic acid (MOPS) buffer.
V. The saccharide coupled microsphere obtained by the method as disclosed in any one of embodiments I to III.
VI. The saccharide coupled microsphere as disclosed in embodiment IV, wherein the saccharide coupled microsphere has Mean Fluorescence Intensity/MFI value in range of 200 to 20,000.
VII. The method as disclosed in any one of embodiments I to IV, wherein the saccharide is bacterial saccharide including Group A Streptococcus, Group B Streptococcus, Streptococcus pneumoniae/pneumococcus, Haemophilus bacteria, Haemophilus influenzae bacteria, Haemophilus influenzae type b bacteria (Hib), Salmonella, Typhoidal salmonella, Non-typhoidal salmonella, Salmonella typhi, Salmonella typhimurium, Salmonella paratyphi, Streptococcus pyogenes, Streptococcus agalactiae, Shigella, Shigella dysenteriae, Shigella flexneri, Shigella sonnei, Escherichia coli, or Neisseria meningitidis/meningococcus saccharide.
VIII. The method as disclosed in embodiment VII, wherein the saccharide is Streptococcus pneumoniae polysaccharide.
IX. The method as disclosed in embodiment VIII, wherein Streptococcus pneumoniae polysaccharide is selected from serotypes 1, 2, 3, 4, 5, 6, 6A, 6B, 6C, 6D, 7, 7B, 7C, 7F, 8, 9, 9A, 9L, 9N, 9V, 10, 10A, 10B, 10C, 10F, 10X, 11, 11A, 11B, 11C, 11F, 12, 12A, 12B, 12F, 13, 14, 15, 15A, 15B, 15BC, 15C, 15F, 16, 16F, 17, 17A, 17F, 18, 18A, 18B, 18C, 18F, 19, 19A, 19B, 19F, 20, 20A, 20B, 20F, 21, 22, 22A, 22F, 23, 23A, 23B, 23F, 24, 24F, 25, 25F, 26, 27, 28, 28A, 28F, 29, 30, 31, 32, 33, 33A, 33B, 33C, 33D, 33F, 34, 35, 35A, 35B, 35C, 35D, 35F, 36, 37, 38, 39, 40, 41, 42, 43, 44, 45, 46, 47, and/or 48.
X. The method as disclosed in embodiment VIII, wherein the Streptococcus pneumoniae polysaccharide is selected from serotypes 1, 2, 3, 4, 5, 6A, 6B, 7F, 8, 9V, 10A, 11A, 12F, 14, 15B, 18C, 19A, 19F, 22F, 23F, 24F and/or 33F.
XI. The method as disclosed in any one of embodiments VIII to X, wherein the Streptococcus pneumoniae polysaccharide has a molecular size in range of 50 kDa to 3000 kDa, preferably in range of 50 kDa to 1500 kDa, is diluted with PBST buffer at a pH in range of 3.0 to 9.0, preferably at pH in range of 4.0 to 7.0, and has concentration in range of 0.05 mg/mL to 50.0 mg/mL, preferably in range of 0.05 mg/mL to 50 mg/mL, more preferably in range of 0.05 mg/mL to 10 mg/mL.
XII. The method as disclosed in any one of embodiments VIII to XI, wherein the Streptococcus pneumoniae polysaccharide is mixed with the microsphere and incubated at temperature in range of 20 to 40° C., preferably in range of 23° C. to 39° C., for incubation time of 60 mins to 120 mins.
XIII. A Streptococcus pneumoniae saccharide coupled microsphere obtained by the method as disclosed in any one of embodiments VIII to XII.
XIV. The method as disclosed in any one of embodiments I to IV, wherein the saccharide is Haemophilus influenzae type b bacteria (Hib) polysaccharide.
XV. The method as disclosed in embodiment XIV, wherein the Haemophilus influenzae type b bacteria (Hib) polysaccharide has a molecular size in range of 0.05 kDa to kDa, is diluted with PBST buffer at a pH in range of 4 to 6 and has concentration of 1.0 μg/ml to 50.0 μg/ml.
XVI. The method as disclosed in any one of embodiments XIV or XV, wherein the mixing of the Haemophilus influenzae type b bacteria (Hib) polysaccharide with the microsphere is done for incubation temperature of 20° C. to 30° C., for incubation time of 60 mins to 120 mins.
XVII. A Haemophilus influenzae type b bacteria (Hib) saccharide coupled microsphere obtained by the method as disclosed in any one of embodiments XIV to XVI.
XVIII. The method as disclosed in any one of embodiments I to IV, wherein the saccharide is Neisseria meningitidis saccharide.
XIX. The method as disclosed in embodiment XVIII, wherein the polysaccharide is Neisseria meningitidis polysaccharide serotypes selected from meningococcal serotypes A (type I and III), B (type II), B6, B16, C (type II-alpha), D (type IV), Z′/E, E29, H, I, K, K454, L, M, W135, X, Y, Z.
XX. The method as disclosed in any one of embodiments XVIII to XIX, wherein the Neisseria meningitidis polysaccharide has a molecular size in range of 75 kDa to 3000 kDa, is diluted with PBST buffer at a pH in range of 4.0 to 7.0 and has concentration of 0.01 mg/mL to 10.0 mg/mL.
XXI. The method as disclosed in any one of embodiments XVIII or XX, wherein the mixing of the Neisseria meningitidis polysaccharide with the microsphere is done for incubation temperature of 20° C. to 40° C. for incubation time of 60 mins to 120 mins.
XXII. A Neisseria meningitidis polysaccharide saccharide coupled microsphere obtained by the method as disclosed in any one of embodiments XVIII to XXI.
XXIII. A method of evaluating immunogenicity of immunogenic composition, the method comprising;
   a. providing a test sample corresponding to the saccharide in the immunogenic composition;
   b. providing the saccharide coupled microsphere corresponding to the saccharide in the immunogenic composition;
   c. evaluating the immunogenicity of the immunogenic composition by antigen content determination, or estimating antibody concentration (IgG), or identity assay, free polysaccharide estimation using the test sample and the saccharide coupled microsphere,
wherein the saccharide coupled microsphere is obtained by the method as disclosed in any one of embodiments I to IV.
XXIV. An antigen content determination method using the saccharide coupled microsphere obtained by the method as disclosed in any one of embodiments I to IV.
XXV. An identity assay method using the saccharide coupled microsphere obtained by the method as disclosed in any one of embodiments I to IV.
XXVI. A free polysaccharide estimation method using the saccharide coupled microsphere obtained by the method as disclosed in any one of embodiments I to IV.
XXVII. A method of estimating antibody concentration (IgG) in sera sample, the method using the saccharide coupled microsphere obtained by the method as disclosed in any one of embodiments I to IV.
XXVIII. The method of estimating antibody concentration (IgG) in sera sample as disclosed in embodiment XXVII, wherein the sera sample includes a human sera sample and an animal sera sample.
XXIX. An apparatus comprising the saccharide coupled microsphere obtained by the method as disclosed in any one of embodiments I to IV.
XXX. The saccharide coupled microsphere as and when used in assay or evaluation of a vaccine or an immunogenic composition, wherein the saccharide coupled microsphere is obtained by the method as disclosed in any one of embodiments I to IV.
XXXI. A method of coupling a saccharide to a microsphere, the method comprising:
   a. providing at least one microsphere;
   b. providing at least one saccharide; and
   c. mixing the at least one microsphere with the at least one saccharide to form a saccharide coupled microsphere;
wherein the saccharide has a size in the range of 0.01 kDa to 3000 kDa.
XXXII. The method of coupling a saccharide to a microsphere as disclosed in any one of previous embodiments, wherein the microsphere is either magnetic or non-magnetic.
XXXIII. The method of coupling a saccharide to a microsphere as disclosed in any one of previous embodiments, wherein the microsphere is activated before mixing with the saccharide.
XXXIV. The method of coupling a saccharide to a microsphere as disclosed in any one of previous embodiments, wherein the microsphere is activated using a bead reagent having metal ions.
XXXV. The method of coupling a saccharide to a microsphere as disclosed in any one of previous embodiments, wherein the microsphere is activated using a nickel based bead reagent.
XXXVI. The method of coupling a saccharide to a microsphere as disclosed in any one of previous embodiments, wherein the saccharide is native saccharide or size reduced saccharide.
XXXVII. The method of coupling a saccharide to a microsphere as disclosed in any one of previous embodiments, wherein the saccharide is size reduced saccharide.
XXXVIII. The method of coupling a saccharide to a microsphere as disclosed in any one of previous embodiments, wherein the saccharide is diluted with PBST buffer before mixing.
XXXIX. The method of coupling a saccharide to a microsphere as disclosed in any one of previous embodiments, wherein the mixing is aqueous based reaction.
XL. The method of coupling a saccharide to a microsphere as disclosed in any one of previous embodiments, wherein the method allows development of electron donor sites forming electrostatic attachment of the saccharide on to the microsphere, wherein the saccharide is intact.
XLI. The method of coupling a saccharide to a microsphere as disclosed in any one of previous embodiments, wherein the saccharide concentration, the size of polysaccharide, mixing incubation temperature, mixing incubation time, pH of buffer is optimised to allow development of electron donor sites forming electrostatic attachment of the saccharide on to the microsphere, wherein the saccharide is intact.
XLII. The method of coupling a saccharide to a microsphere as disclosed in any one of previous embodiments, wherein
   optionally, the mixing is performed with the coupling ratio of the microsphere to the saccharide is in range of 50 to 12500, and/or
   optionally, the metal ions are adsorbed on surface of the microsphere during activation of the microsphere before mixing with the saccharide, and/or
   optionally, the saccharide coupled microsphere is formed by non-covalent electrostatic coupling, and/or
   optionally, the saccharide remains intact, retains epitope confirmation and the saccharide coupled microsphere is stable.
XLIII. The method of coupling a saccharide to a microsphere as disclosed in any one of previous embodiments, wherein
   the mixing is performed with the coupling ratio of the microsphere to the saccharide is in range of 50 to 12500, and
   the saccharide coupled microsphere is formed by non-covalent electrostatic coupling.
XLIV. The method of coupling a saccharide to a microsphere as disclosed in any one of previous embodiments, wherein
   the mixing is performed with the coupling ratio of the microsphere to the saccharide is in range of 50 to 12500,
   the metal ions are adsorbed on surface of the microsphere during activation of the microsphere before mixing with the saccharide,
   the saccharide coupled microsphere is formed by non-covalent electrostatic coupling,
   the saccharide remains intact, retains epitope confirmation and the saccharide coupled microsphere is stable.
XLV. The method of coupling a saccharide to a microsphere as disclosed in any one of previous embodiments, wherein the saccharide is pneumococcal polysaccharide saccharide selected from a group of polysaccharides associated with Streptococcus pneumoniae/pneumococcal strains associated with global Streptococcus pneumoniae/pneumococcal sequence cluster (GPSC) selected from GPSC 1 to 131.
XLVI. The method of coupling a saccharide to a microsphere as disclosed in any one of previous embodiments, wherein the saccharide is associated Haemophilus bacteria type a, type b, type c, type d, type e, or type f.
XLVII. The method of coupling a saccharide to a microsphere as disclosed in any one of previous embodiments, wherein the saccharide is Haemophilus influenzae type b bacteria (Hib) saccharide selected from a group of saccharides associated with type I and type II Hib strain.
XLVIII. The method of coupling a saccharide to a microsphere as disclosed in any one of previous embodiments, wherein the polysaccharide is Haemophilus influenzae type b bacteria (Hib) polysaccharide selected from a group of polysaccharides associated with variation of hcsA gene.
XLIX. The method of coupling a saccharide to a microsphere as disclosed in any one of previous embodiments, wherein the polysaccharide is meningococcal polysaccharide selected from a group of polysaccharides associated with variation in IpxL1, fHbp, and tps genes.
L. An apparatus comprising the saccharide coupled microsphere obtained by the method as disclosed in any one of previous embodiments.

### Examples

The present invention is further illustrated in combination with the following examples. These examples are provided to exemplify the present invention but are not intended to restrict the scope of the presently claimed invention in any way. The terms and abbreviations in the examples have their common meanings. For example, “%”, “Eq. wt.”, “Eq.”, “° C.”, “wt. %”, “% w/w”, “% w/v” and “gm” represent “percentage”, “Equivalent Weight”, “Equivalents”, “degree Celsius”, “percent by weight”, “percent weight by weight”, “percent weight by volume” and “gram” respectively.

### Reagents/Solutions/Instruments/Equipment:

A. Coupling reagents/Chemicals:
Activation reagent: Anteotech
Storage buffer: PBST with 1% BSA (SIIPL—Inhouse). BSA from Sigma Aldrich, India
Beads: Magnetic beads (Luminex Corporation), MicroPlex® Microspheres, MagPlex® Microspheres
Trypan Blue (Sigma)
PBST (Phosphate-buffered saline with Tween 20) buffer: Sodium Chloride (Qualigens), Potassium Chloride (Qualigens), Di-sodium hydrogen phosphate (Merck), Potassium dihydrogen phosphate (Sigma-Aldrich), Tween 20 (SDFCL), Sodium hydroxide (Qualigens), Hydrochloric acid (Sigma-Aldrich).
Blocking Buffer: BSA/Bodvine Serum Albumin (Sigma Aldrich, India)
PBS Buffer: Sodium Chloride (Qualigens), Potassium Chloride (Qualigens), Di-sodium hydrogen phosphate (Merck), Potassium dihydrogen phosphate (Sigma-Aldrich), Sodium hydroxide (Qualigens), Hydrochloric acid (Sigma-Aldrich).
   B. Testing reagents/Chemicals:
10×PBS buffer
20# W/V Sodium azide (NaN3): Sodium Azide (Sigma-Aldrich),
Luminex Assay Buffer: 1×PBS, BSA (Sigma Aldrich), Tween 20 (SDFCL), Sodium hydroxide/NaOH (Qualigens), Citric acid (Qualigens).
Formulation Buffer (Aluminium Phosphate buffer): 20 mM Histidine, Succinate buffer, Thiomerseal, Tween 20, Adjuvant.
Phycoerythrin: Human Phycoerythrin, Rat Phycoerythrin, Rabbit Phycoerythrin, Mice Phycoerythrin from Jackson Immunoresearch, USA.
Reference standard: Respective Streptococcus pneumoniae (Pneumococcal) monovalent conjugate bulks of serotype 1, 2, 3, 4, 5, 6A, 6B, 7F, 8, 9V, 10A, 11A, 12F, 14, 15B, 18C, 19A, 19F, 22F, 23F, 33F, and 24F. (Serotype 24F is from Kempegowda Institute of Medical Sciences, KIMS, Bangalore, India. Rest all Pneumococcal serotypes are from Center for Disease Control and Prevention (CDC), Atlanta, USA)
Reference standard: Haemophilus influenzae type b saccharides. The Hib strain 760705 was obtained from Netherlands Vaccines Institute (NVI, The Netherlands)
Reference standard: Neisseria meningitis (A, C, W, Y, X) saccharides. Neisseria meningitidis A (strain M1027 from SynCo Biopartners, Netherlands), Neisseria meningitidis C (strain C11(60E) from CBER/FDA, USA), Neisseria meningitidis W (strain S877 from CBER/FDA, USA), Neisseria meningitidis Y (strain M10659 from CDC, USA), Neisseria meningitidis (strain X M8210 from CBER/FDA, USA)
Monoclonal Antibody: Antibody against Pneumococcal monovalent conjugate bulks of serotype 1, 2, 3, 4, 5, 6A, 6B, 7F, 8, 9V, 10A, 11A, 12F, 14, 15B, 18C, 19A, 19F, 22F, 23F, 33F, and 24F. (Staten Serum Institute)
Carboxylated coupled beads: Carboxylated beads coupled with serotypes Pneumococcal monovalent conjugate bulks of serotype 1, 2, 3, 4, 5, 6A, 6B, 7F, 8, 9V, 10A, 11A, 12F, 14, 15B, 18C, 19A, 19F, 22F, 23F, 33F, and 24F. (SIIPL-Inhouse).
For animal testing license: 41/PO/RcBi/S/99/CPCSEA dated: Aug. 3, 2022
Ethical committee approval: 17/30-09/2024-MT.
No any external sample were used for human, the international human reference std i.e. NIBSC (WHO) international reference standard i.e. 007Sp were used
Rat Tox sera samples proposed from Advenous laboratory which were vaccinated with PCV 21 valent at Advenous Lab.

### C. Instruments:

Automated Cell counter: Bio-Rad
Inverted microscope: Nikon
Analytical weighing balance: Sartorius
pH meter: Thermo Orian
Centrifuge: Thermo scientific
Shaker Incubator: Titramax
Protein Suspension array system (Bioplex-200): Bio-Rad
Vaccum manifold: Millipore
Sonicator: Amkette industries
Micropipette, Multi-channel (30-300 μL and 10-100 μL), Single-channel (0.5-10 μL, 10-100 μL, 20-200 L, 100-1000 μL and 500-5000 μL): All from Eppendorf
Magnetic separator: Bio-Rad

### Example 1: Dilution of Native Polysaccharides (Streptococcal Pneumoniae Polysaccharides)

Native polysaccharide available stock concentration range for all the serotypes was between to 10 mg/ml and for size reduced polysaccharide between 11 to 15 mg/ml. As per Table 1 below, each serotype required the optimized concentration range, hence stock of polysaccharide was diluted in the mentioned respective buffer condition prior to coupling.

TABLE 1
DILUTION OF NATIVE POLYSACCHARIDES

| | | | Conc. of PS | PnPS |
|---|---|---|---|---|
| Sr. | | | require for | Reconstitution |
| No. | Serotype | Chemical composition | coupling | Solution/Buffer |
| 1 | 1 | Nitrogen, Phosphorus, Uronic acids, | 0.5 to 1.0 | PBST (pH 5.5 ± |
| | | O-acetyl | mg/mL | 0.5), 5-6 |
| 2 | 2 | Nitrogen, Phosphorus, Uronic acids, | 1.5 to 2.0 | PBST (pH 4.5 ± |
| | | Methyl-pentoses | mg/mL | 0.5), 4-5 |
| 3 | 4 | Nitrogen, Phosphorus, O-acetyl, | 0.5 to 1.0 | PBST (pH 6.5 ± |
| | | Hexosamines | mg/mL | 0.5), 6-7 |
| 4 | 5 | Nitrogen, Phosphorus, Uronic acids, | 2.0 to 2.5 | PBST (pH 6.5 ± |
| | | Hexosamines | mg/mL | 0.5), 6-7 |
| 5 | 6A | Nitrogen, Phosphorus, Methyl | 1.5 to 2.0 | PBST (pH 4.5 ± |
| | | pentose | mg/mL | 0.5), 4-5 |
| 6 | 6B | Nitrogen, Phosphorus, Methyl | 0.5 to 1.0 | PBST (pH 5.5 ± |
| | | pentose | mg/mL | 0.5), 5-6 |
| 7 | 7F | Nitrogen, Phosphorus, | 0.5 to 1.0 | PBST (pH 6.5 ± |
| | | Hexosamines, Methyl pentose, O- | mg/mL | 0.5), 6-7 |
| | | acetyl | | |
| 8 | 8 | Nitrogen, Phosphorus, Uronic acid | 1.5 to 2.0 | PBST (pH 4.5 ± |
| | | | mg/mL | 0.5), 4-5 |
| 9 | 9V | Nitrogen, Phosphorus, Uronic acid, | 0.5 to 1.0 | PBST (pH 5.5 ± |
| | | Hexosamines, O-acetyl | mg/mL | 0.5), 5-6 |
| 10 | 10A | Nitrogen, Phosphorus, | 0.5 to 1.0 | PBST (pH 6.5 ± |
| | | Hexosamines | mg/mL | 0.5), 6-7 |
| 11 | 11A | Nitrogen, Phosphorus, O-acetyl | 2.0 to 2.5 | PBST (pH 5.5 ± |
| | | | mg/mL | 0.5), 5-6 |
| 12 | 12F | Nitrogen, Phosphorus, Uronic acid, | 0.5 to 1.0 | PBST (pH 6.5 ± |
| | | Hexosamines, Methyl pentose, | mg/mL | 0.5), 6-7 |
| 13 | 14 | Nitrogen, Phosphorus, | 2.0 to 2.5 | PBST (pH 6.5 ± |
| | | Hexosamines | mg/mL | 0.5), 6-7 |
| 14 | 15B | Nitrogen, Phosphorus, | 2.0 to 2.5 | PBST (pH 5.5 ± |
| | | Hexosamines, O-acetyl | mg/mL | 0.5), 5-6 |
| 15 | 18C | Nitrogen, Phosphorus, Methyl | 2.0 to 2.5 | PBST (pH 6.5 ± |
| | | pentose, O-acetyl | mg/mL | 0.5), 6-7 |
| 16 | 19A | Nitrogen, Phosphorus, | 0.5 to 1.0 | PBST (pH 5.5 ± |
| | | Hexosamines, Methyl pentose | mg/mL | 0.5), 5-6 |
| 17 | 19F | Nitrogen, Phosphorus, | 0.5 to 1.0 | PBST (pH 5.5 ± |
| | | Hexosamines, Methyl pentose | mg/mL | 0.5), 5-6 |
| 18 | 22F | Nitrogen, Phosphorus, Uronic acid, | 2.0 to 2.5 | PBST (pH 5.5 ± |
| | | Methyl pentose, O-acetyl | mg/mL | 0.5), 5-6 |
| 19 | 23F | Nitrogen, Phosphorus, Methyl | 2.0 to 2.5 | PBST (pH 4.5 ± |
| | | pentose, O-acetyl | mg/mL | 0.5), 4-5 |
| 20 | 24F | Nitrogen, Phosphorus, | 0.5 to 1.0 | PBST (pH 6.5 ± |
| | | Hexosamines, Methyl pentose | mg/mL | 0.5), 6-7 |
| 21 | 33F | Nitrogen, Phosphorus, O-acetyl | 2.0 to 2.5 | PBST (pH 6.5 ± |
| | | | mg/mL | 0.5), 6-7 |

### Example 2: Composition Associated with Streptococcal Polysaccharide Coupled Microspheres

Each Streptococcal polysaccharide saccharide contain different functional groups, sizes and structure. The specific conditions applied to different Saccharides during dilution of the saccharide stock and further conjugation to the beads gave the optimal MFI's to positive samples. The following table 2 represents these conditions.

TABLE 2
SEROTYPES AND COMPOSITIONS

| | | Steps applied during | | | | | |
|---|---|---|---|---|---|---|---|
| | | dilution of PnPS stock | | | Steps applied | | |
| | | Conc. Of | Size | | during coupling | | |
| | Chemical | PS require | of Ps | Dilution of | for incubation | | |
| SR | composition | for coupling | (kDa) | PnPS stocks | Time | | Temp |
| 1 | Nitrogen, | 0.5 to 1.0 | 80 | PBST (pH | 1 | Hr | 25° C. |
| | Phosphorus, | mg/mL | | 5.5 ± 0.5), | | | |
| | Uronic acids, | | | 5-6 | | | |
| | O-acetyl | | | | | | |
| 2 | Nitrogen, | 1.5 to 2.0 | 1011 | PBST (pH | 2 | Hr | 37° C. |
| | Phosphorus, | mg/mL | | 4.5 ± 0.5), | | | |
| | Uronic acids, | | | 4-5 | | | |
| | Methyl-pentoses | | | | | | |
| 4 | Nitrogen, | 0.5 to 1.0 | 87 | PBST (pH | 1.5 | Hr | 25° C. |
| | Phosphorus, | mg/mL | | 6.5 ± 0.5), | | | |
| | O-acetyl, | | | 6-7 | | | |
| | Hexosamines | | | | | | |
| 5 | Nitrogen, | 2.0 to 2.5 | 75 | PBST (pH | 1.5 | Hr | 25° C. |
| | Phosphorus, | mg/mL | | 6.5 ± 0.5), | | | |
| | Uronic acids, | | | 6-7 | | | |
| | Hexosamines | | | | | | |
| 6A | Nitrogen, | 1.5 to 2.0 | 551 | PBST (pH | 2 | Hr | 37° C. |
| | Phosphorus, | mg/mL | | 4.5 ± 0.5), | | | |
| | Methyl pentose | | | 4-5 | | | |
| 6B | Nitrogen, | 0.5 to 1.0 | 107 | PBST (pH | 1 | Hr | 25° C. |
| | Phosphorus, | mg/mL | | 5.5 ± 0.5), | | | |
| | Methyl pentose | | | 5-6 | | | |
| 7F | Nitrogen, | 0.5 to 1.0 | 135 | PBST (pH | 1.5 | Hr | 25° C. |
| | Phosphorus, | mg/mL | | 6.5 ± 0.5), | | | |
| | Hexosamines, | | | 6-7 | | | |
| | Methyl pentose, | | | | | | |
| | O-acetyl | | | | | | |
| 8 | Nitrogen, | 1.5 to 2.0 | 565 | PBST (pH | 2 | Hr | 37° C. |
| | Phosphorus, | mg/mL | | 4.5 ± 0.5), | | | |
| | Uronic acid | | | 4-5 | | | |
| 9V | Nitrogen, | 0.5 to 1.0 | 108 | PBST (pH | 1.5 | Hr | 25° C. |
| | Phosphorus, | mg/mL | | 5.5 ± 0.5), | | | |
| | Uronic acid, | | | 5-6 | | | |
| | Hexosamines, | | | | | | |
| | O-acetyl | | | | | | |
| 10A | Nitrogen, | 0.5 to 1.0 | 119 | PBST (pH | 1 | Hr | 25° C. |
| | Phosphorus, | mg/mL | | 6.5 ± 0.5), | | | |
| | Hexosamines | | | 6-7 | | | |
| 11A | Nitrogen, | 2.0 to 2.5 | 100 | PBST (pH | 1.5 | Hr | 25° C. |
| | Phosphorus, | mg/mL | | 5.5 ± 0.5), | | | |
| | O-acetyl | | | 5-6 | | | |
| 12F | Nitrogen, | 0.5 to 1.0 | 99 | PBST (pH | 1.5 | Hr | 25° C. |
| | Phosphorus, | mg/mL | | 6.5 ± 0.5), | | | |
| | Uronic acid, | | | 6-7 | | | |
| | Hexosamines, | | | | | | |
| | Methyl pentose, | | | | | | |
| 14 | Nitrogen, | 2.0 to 2.5 | 143 | PBST (pH | 1.5 | Hr | 25° C. |
| | Phosphorus, | mg/mL | | 6.5 ± 0.5), | | | |
| | Hexosamines | | | 6-7 | | | |
| 15B | Nitrogen, | 2.0 to 2.5 | 136 | PBST (pH | 1.5 | Hr | 25° C. |
| | Phosphorus, | mg/mL | | 5.5 ± 0.5), | | | |
| | Hexosamines, | | | 5-6 | | | |
| | O-acetyl | | | | | | |
| 18C | Nitrogen, | 2.0 to 2.5 | 79 | PBST (pH | 1.5 | Hr | 25° C. |
| | Phosphorus, | mg/mL | | 6.5 ± 0.5), | | | |
| | Methyl pentose, | | | 6-7 | | | |
| | O-acetyl | | | | | | |
| 19A | Nitrogen, | 0.5 to 1.0 | 91 | PBST (pH | 1.5 | Hr | 25° C. |
| | Phosphorus, | mg/mL | | 5.5 ± 0.5), | | | |
| | Hexosamines, | | | 5-6 | | | |
| | Methyl pentose | | | | | | |
| 19F | Nitrogen, | 0.5 to 1.0 | 85 | PBST (pH | 1.5 | Hr | 25° C. |
| | Phosphorus, | mg/mL | | 4.5 ± 0.5), | | | |
| | Hexosamines, | | | 4-5 | | | |
| | Methyl pentose | | | | | | |
| 22F | Nitrogen, | 2.0 to 2.5 | 106 | PBST (pH | 1 | Hr | 25° C. |
| | Phosphorus, | mg/mL | | 5.5 ± 0.5), | | | |
| | Uronic acid, | | | 5-6 | | | |
| | Methyl pentose, | | | | | | |
| | O-acetyl | | | | | | |
| 23F | Nitrogen, | 2.0 to 2.5 | 94 | PBST (pH | 1 | Hr | 25° C. |
| | Phosphorus, | mg/mL | | 4.5 ± 0.5), | | | |
| | Methyl pentose, | | | 4-5 | | | |
| | O-acetyl | | | | | | |
| 24F | Nitrogen, | 0.5 to 1.0 | 81 | PBST (pH | 1 | Hr | 25° C. |
| | Phosphorus, | mg/mL | | 6.5 ± 0.5), | | | |
| | Hexosamines, | | | 6-7 | | | |
| | Methyl pentose | | | | | | |
| 33F | Nitrogen, | 2.0 to 2.5 | 191 | PBST (pH | 1 | Hr | 25° C. |
| | Phosphorus, | mg/mL | | 6.5 ± 0.5), | | | |
| | O-acetyl | | | 6-7 | | | |

### Example 3: Streptococcus pneumoniae/Pneumococcal Serotype—Type 1

Efficient coupling was outcome of correct saccharide size, concentration and pH Efficiently coupled beads have following characteristics:
a) Higher sensitivity
b) Less noise/background
c) Good precision (CV among duplicates) and accuracy (back fitted recoveries) The specific conditions applied to Pneumococcus type 1 serotype Saccharides during dilution of the saccharide stock and further conjugation to the beads/microspheres gave the optimal MFI's to positive samples.

TABLE 3a
Serotype 1 conditions of pH and Concentration of Polysaccharides,
molecular size, incubation temperature and time.

| | | | Molecular | |
|---|---|---|---|---|
| Type | | Concentra- | size | Incubation time, |
| 1 | pH (PBST) | tion of Ps | (kDa) | temperature |
| A | (pH 4.5 ± 0.5), 4-5 | 0.1 mg/mL | 80 | 1 Hr/25deg C. |
| B | (pH 4.5 ± 0.5), 4-5 | 0.3 mg/mL | 80 | 1 Hr/25deg C. |
| C | (pH 5.5 ± 0.5), 5-6 | 0.5 mg/mL | 80 | 1 Hr/25deg C. |
| D | (pH 5.5 ± 0.5), 5-6 | 1.0 mg/mL | 80 | 1 Hr/25deg C. |

TABLE 3b
Serotype 1 conditions of pH and Concentration of Polysaccharides,
molecular size, incubation temperature and time.

| | | | Molecular | |
|---|---|---|---|---|
| | | Concentration | size | Incubation time, |
| Condition | pH (PBST) | of PS | (kDa) | Temperature |
| A | (pH 4.5 ± 0.5), 4-5 | 0.5 to 1.0 mg/mL | 540 | 1 Hr/25deg C. |
| B | (pH 5.5 ± 0.5), 5-6 | 0.5 to 1.0 mg/mL | 540 | 1 Hr/25deg C. |
| C | (pH 4.5 ± 0.5), 4-5 | 0.5 to 1.0 mg/mL | 80 | 1 Hr/25deg C. |
| D | (pH 5.5 ± 0.5), 5-6 | 0.5 to 1.0 mg/mL | 80 | 1 Hr/25deg C. |

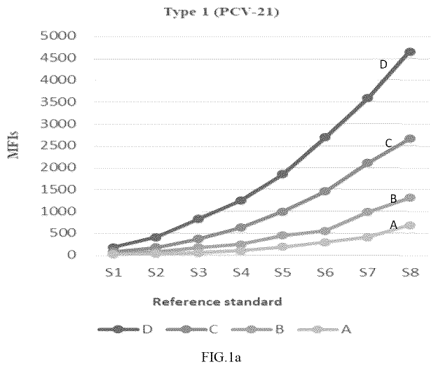

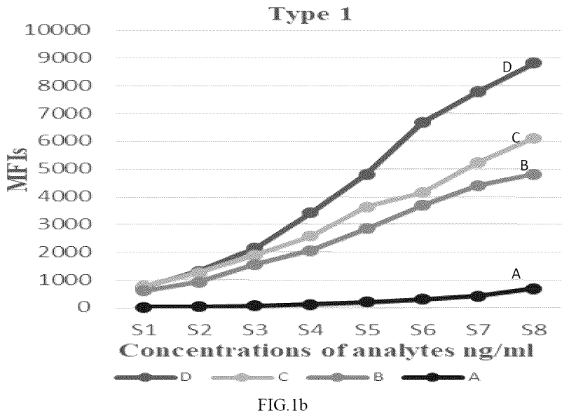

See FIGS. 1a and 1b & TABLE 3a and 3b for details of MFI value against Type 1 serotype in conditions of pH and Concentration of Polysaccharides, molecular size, incubation temperature and time.

Inference: Four different conditions (A, B, C, D) were studied to obtain the maximum signal (MFI) of type 1 Saccharide coupled microspheres (PS coupled beads). During coupling, the pH range, Saccharide (Ps) Concentration, Molecular size and Incubation time was critically studied. From the above table the maximum MFI signal were observed for condition-D compared with other 3 conditions. If Higher the MFI values is directly proportional to the higher sensitivity of the assay, hence parameters mentioned in the condition-D are optimal for coupling serotype serotype-1 antigen to microsphere beads.

### Example 4: Streptococcus pneumoniae/Pneumococcal Type 2 Serotype

The specific conditions applied to Pneumococcus type 2 serotype Saccharides during dilution of the saccharide stock and further conjugation to the beads/microspheres gave the optimal MFI's to positive samples.

TABLE 4a
STREPTOCOCCUS PNEUMONIAE/PNEUMOCOCCAL TYPE 2 Serotype
conditions of pH and Concentration of Polysaccharides,
molecular size, incubation temperature and time.

| | | | | Molecular | |
|---|---|---|---|---|---|
| Type | | | Concentration | size | Incubation time, |
| 2 | Buffer | pH | of Ps | (kDa) | temperature |
| A | PBST | PBST (pH 4.5 ± 0.5), 4-5 | 0.5 to 1.0 mg/mL | 80 | 1 Hr/25deg C. |
| B | PBST | PBST (pH 4.5 ± 0.5), 4-5 | 0.5 to 1.0 mg/mL | 80 | 1 Hr/37deg C. |
| C | PBST | PBST (pH 4.5 ± 0.5), 4-5 | 1.5 to 2.0 mg/mL | 80 | 2 Hr/37deg C. |
| D | PBST | PBST (pH 4.5 ± 0.5), 4-5 | 1.5 to 2.0 mg/mL | 1011 | 2 Hr/37deg C. |

TABLE 4b
STREPTOCOCCUS PNEUMONIAE/PNEUMOCOCCAL TYPE 2 Serotype
conditions of pH and Concentration of Polysaccharides,
molecular size, incubation temperature and time.

| | | | Molecular | |
|---|---|---|---|---|
| | | Concentration | size | Incubation time, |
| Condition | pH | of PS | (kDa) | temperature |
| A | PBST (pH 4.5 ± 0.5), 4-5 | 0.5 to 1.0 mg/mL | 95 | 1 Hr/25deg C. |
| B | PBST (pH 4.5 ± 0.5), 4-5 | 1.5 to 2.0 mg/mL | 95 | 1 Hr/37deg C. |
| C | PBST (pH 4.5 ± 0.5), 4-5 | 1.5 to 2.0 mg/mL | 1011 | 1 Hr/37deg C. |
| D | PBST (pH 4.5 ± 0.5), 4-5 | 1.5 to 2.0 mg/mL | 1011 | 2 Hr/37deg C. |

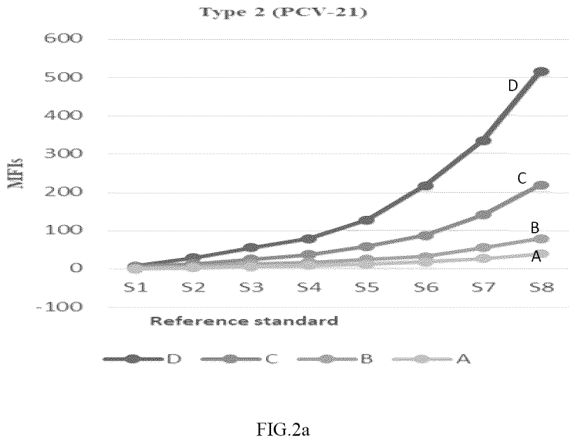

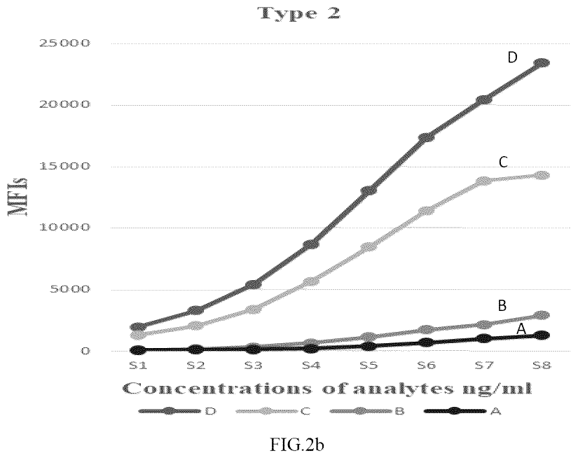

See FIGS. 2a and 2b & TABLE 4a and 4b for details of MFI value against Type 2 serotype in conditions of pH and Concentration of Polysaccharides, molecular size, incubation temperature and time.

The pneumococcal serotype 2 modified saccharide has size 95 kDa and after coupling was observed to be associated with the lesser MFI value compared with the Native saccharide (Size 1011 kDa) which gives a good increase in the MFI value.

Inference: Four different conditions (A, B, C, D) were studied to obtain the maximum signal (MFI) of serotype-2 Saccharide coupled microspheres (PS coupled beads). During coupling, the pH range, Saccharide (Ps) Concentration, Molecular size and Incubation time was critically studied. From the above table the maximum MFI signal were observed for condition-D compared with other 3 conditions. Higher the MFI values is directly proportional to the higher sensitivity of the assay, hence parameters mentioned in the condition-D are optimal for coupling serotype-2 antigen to microsphere beads.

### Example 5: Streptococcus pneumoniae/Pneumococcal Type 4 Serotype

The specific conditions applied to Pneumococcus type 4 serotype Saccharides during dilution of the saccharide stock and further conjugation to the beads/microspheres gave the optimal MFI's to positive samples.

TABLE 5a
STREPTOCOCCUS PNEUMONIAE/PNEUMOCOCCAL TYPE 4 SEROTYPE
conditions of pH and Concentration of Polysaccharides,
molecular size, incubation temperature and time.

| | | | | Molecular | |
|---|---|---|---|---|---|
| Type | | | Concentration | size | Incubation time, |
| 4 | Buffer | pH | of Ps | (kDa) | temperature |
| A | PBST | PBST (pH 4.5 ± 0.5), | 0.5 to 1.0 mg/mL | 87 | 1 Hr/25deg C. |
| | | 4-5 | | | |
| B | PBST | PBST (pH 5.5 ± 0.5), | 0.5 to 1.0 mg/mL | 87 | 1 Hr/25deg C. |
| | | 5-6 | | | |
| C | PBST | PBST (pH 5.5 ± 0.5), | 0.5 to 1.0 mg/mL | 87 | 1.5 Hr/25deg C. |
| | | 5-6 | | | |
| D | PBST | PBST (pH 6.5 ± 0.5), | 0.5 to 1.0 mg/mL | 87 | 1.5 Hr/25deg C. |
| | | 6-7 | | | |

TABLE 5b
STREPTOCOCCUS PNEUMONIAE/PNEUMOCOCCAL TYPE 4 SEROTYPE
conditions of pH and Concentration of Polysaccharides,
molecular size, incubation temperature and time.

| | | | Molecular | |
|---|---|---|---|---|
| | | Concentration | size | Incubation time, |
| Condition | pH | of PS | (kDa) | temperature |
| A | PBST (pH 4.5 ± 0.5), 4-5 | 0.5 to 1.0 mg/mL | 318 | 1 Hr/25deg C. |
| B | PBST (pH 5.5 ± 0.5), 5-6 | 0.5 to 1.0 mg/mL | 318 | 1 Hr/25deg C. |
| C | PBST (pH 6.5 ± 0.5), 6-7 | 0.5 to 1.0 mg/mL | 87 | 1.5 Hr/37deg C. |
| D | PBST (pH 6.5 ± 0.5), 6-7 | 0.5 to 1.0 mg/mL | 87 | 1.5 Hr/25deg C. |

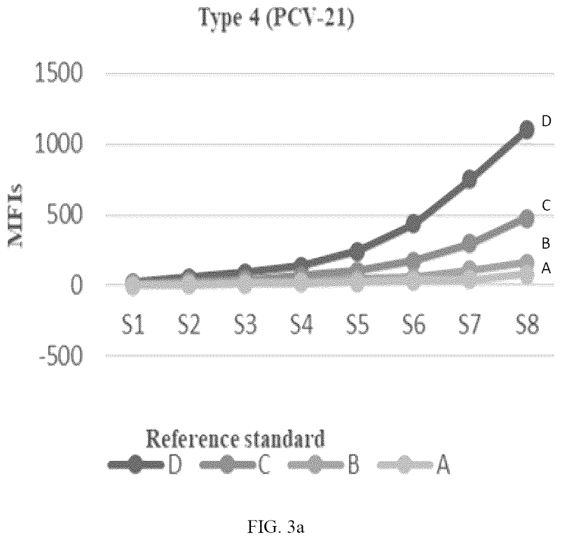

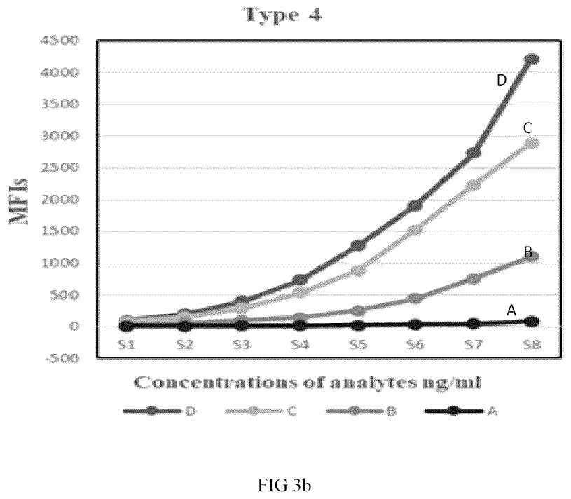

See FIGS. 3a and 3b and TABLE 5a and 5b for details of MFI value against Type 4 serotype in conditions of pH and Concentration of Polysaccharides, molecular size, incubation temperature and time.

Inference: Four different conditions (A, B, C, D) were studied to obtain the maximum signal (MFI) of serotype-4 Saccharide coupled microspheres (PS coupled beads). During coupling, the pH range, Saccharide (Ps) Concentration, Molecular size and Incubation time was critically studied. From the above table the maximum MFI signal were observed for condition-D compared with other 3 conditions. Higher the MFI values is directly proportional to the higher sensitivity of the assay, hence parameters mentioned in the condition-D are optimal for coupling serotype-4 antigen to microsphere beads.

The saccharide coupled microsphere obtained herein above was further qualified using the serological assay (Multiplex-Immunoassay). During qualification stage the system suitability parameters were monitored such as Back fit of the reference standards, duplicate % CV, assay blank and % gradation between the MFI's. To achieve these parameters within the specifications the coupling optimization was performed and no any interference was observed. Accordingly, no interference was observed in the method.

### Example 6: Comparison of Beads of the Present Invention and Conventional Beads

Saccharide coupled microspheres were prepared for PCV-21 serotypes. The coupled beads were evaluated for Human sera i.e. 007SP (007SP is for use in the enzyme-linked immunosorbent assay protocol for quantification of human IgG antibodies specific for Streptococcus pneumoniae capsular polysaccharides (Pn PS ELISA). 007SP is a pooled serum from 278 healthy volunteers following vaccination with 23 valent pneumococcal polysaccharide vaccine to evaluate the coupling procedure. The beads coupled as per present invention was compared with a conventional method (i.e. modified amine coupling) method. It was observed that the obtained MFI's data by metal activated beads were found to give higher sensitivities and higher dynamic range for serotypes 7F, 8, 10A and 24F which was observed higher MFI's than the conventional method. Accordingly, the sensitivity of the assay for the serotypes has increased which has improved the efficiency of the assay.

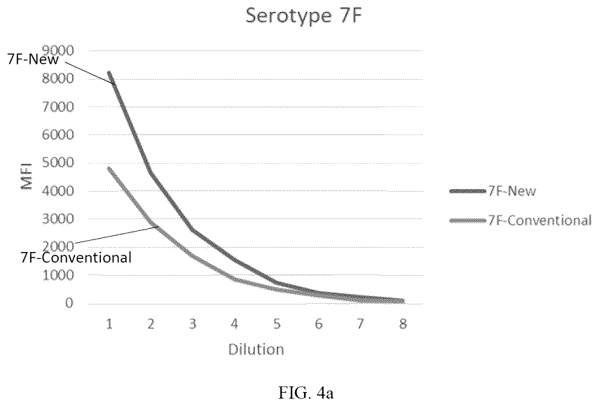

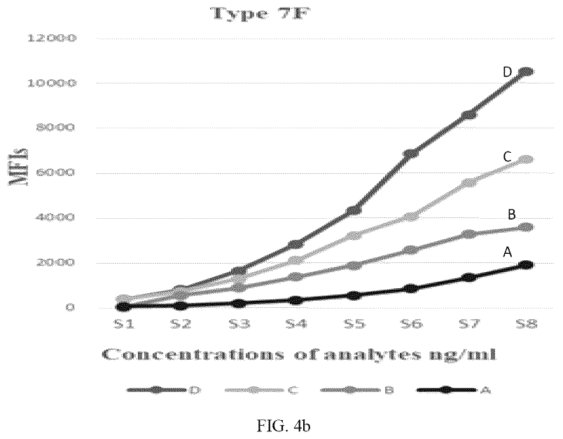

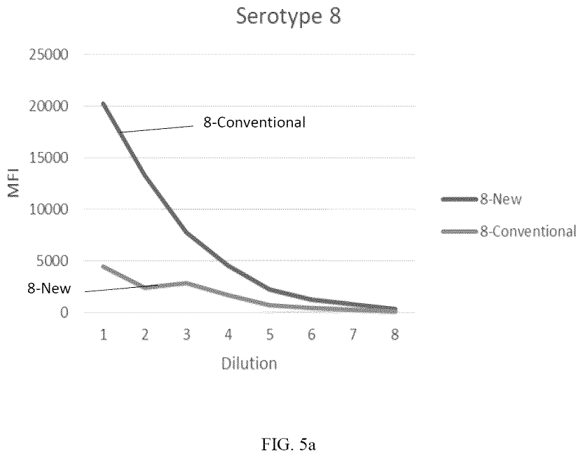

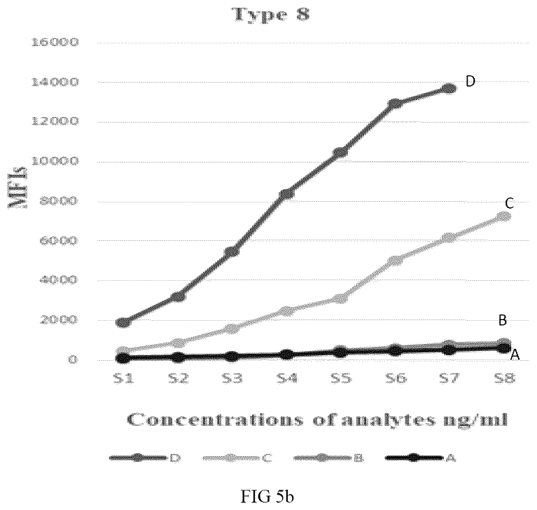

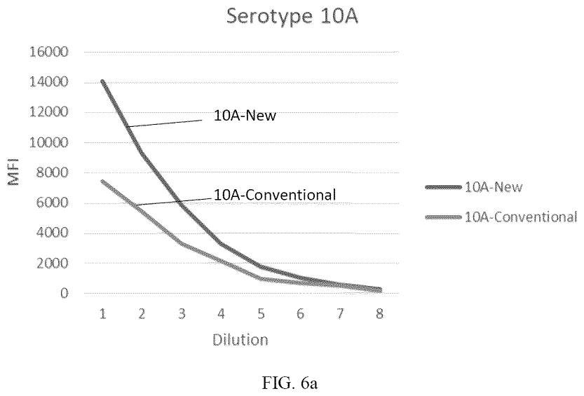

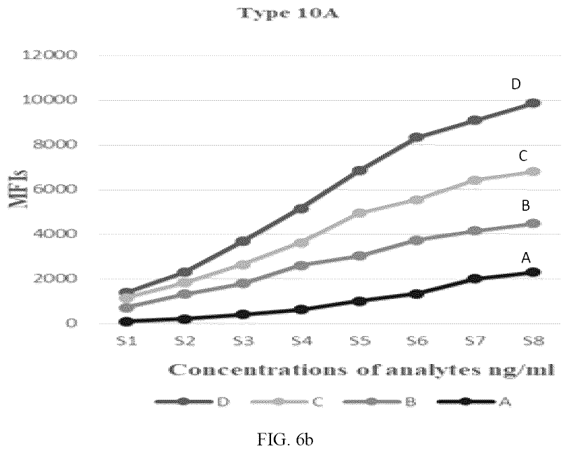

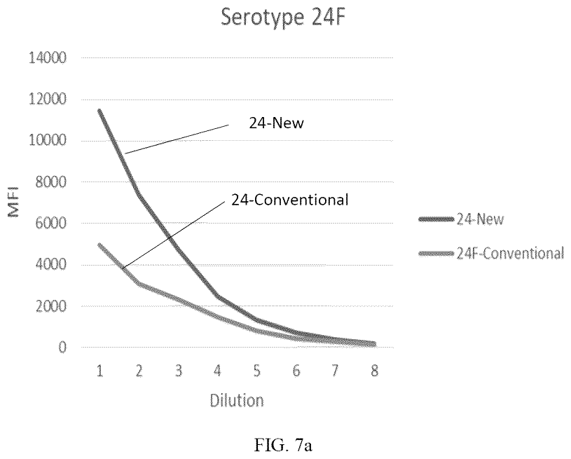

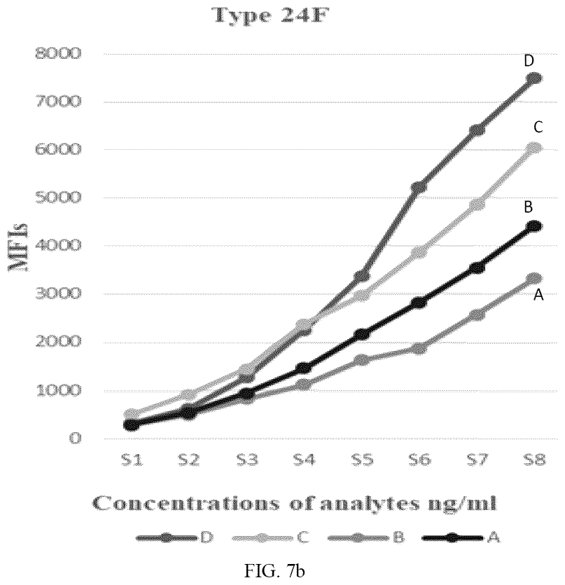

See FIGS. 4a and 4b (for serotype 7), FIGS. 5a and 5b (for serotype 8), FIG. 6 (for serotype 10A), and FIG. 7 (for serotype 24F) for details of MFI value against Type 7F, 8, 10A and 24F serotypes respectively in conditions of pH and Concentration of Polysaccharides, molecular size, incubation temperature and time.

TABLE 6
STREPTOCOCCUS PNEUMONIAE/PNEUMOCOCCAL TYPE 7F SEROTYPE
conditions of pH and Concentration of Polysaccharides,
molecular size, incubation temperature and time.

| | | | Molecular | |
|---|---|---|---|---|
| | | Concentration | size | Incubation time, |
| Condition | pH | of PS | (kDa) | temperature |
| A | PBST (pH 5.5 ± 0.5), 5-6 | 0.5 to 1.0 mg/mL | 511 | 1 Hr/25deg C. |
| B | PBST (pH 5.5 ± 0.5), 5-6 | 0.5 to 1.0 mg/mL | 511 | 1.5 Hr/25deg C. |
| C | PBST (pH 5.5 ± 0.5), 5-6 | 0.5 to 1.0 mg/mL | 135 | 1.5 Hr/25deg C. |
| D | PBST (pH 6.5 ± 0.5), 6-7 | 0.5 to 1.0 mg/mL | 135 | 1.5 Hr/25deg C. |

Inference: Four different conditions (A, B, C, D) were studied to obtain the maximum signal (MFI) of serotype-7F Saccharide coupled microspheres (PS coupled beads). During coupling, the pH range, Saccharide (Ps) Concentration, Molecular size and Incubation time was critically studied. From the above table the maximum MFI signal were observed for condition-D compared with other 3 conditions. Higher the MFI values is directly proportional directly proportional to the higher sensitivity of the assay, hence parameters mentioned in the condition-D are optimal for coupling serotype-7F antigen to microsphere beads.

TABLE 7
STREPTOCOCCUS PNEUMONIAE/PNEUMOCOCCAL TYPE 8 SEROTYPE
conditions of pH and Concentration of Polysaccharides,
molecular size, incubation temperature and time.

| | | | Molecular | |
|---|---|---|---|---|
| | | Concentration | size | Incubation time, |
| Condition | pH | of PS | (kDa) | temperature |
| A | PBST (pH 4.5 ± 0.5), 4-5 | 0.5 to 1.0 mg/mL | 80 | 1 Hr/25deg C. |
| B | PBST (pH 5.5 ± 0.5), 4-5 | 0.5 to 1.0 mg/mL | 80 | 1 Hr/25deg C. |
| C | PBST (pH 4.5 ± 0.5), 4-5 | 1.5 to 2.0 mg/mL | 565 | 1 Hr/37deg C. |
| D | PBST (pH 4.5 ± 0.5), 4-5 | 1.5 to 2.0 mg/mL | 565 | 2 Hr/37deg C. |

Inference: Four different conditions (A, B, C, D) were studied to obtain the maximum signal (MFI) of serotype-10A Saccharide coupled microspheres (PS coupled beads). During coupling, the pH range, Saccharide (Ps) Concentration, Molecular size and Incubation time was critically studied. From the above table the maximum MFI signal were observed for condition-D compared with other 3 conditions. Higher the MFI values is directly proportional to the higher sensitivity of the assay, hence parameters mentioned in the condition-D are optimal for coupling serotype-10A antigen to microsphere beads.

TABLE 8
STREPTOCOCCUS PNEUMONIAE/PNEUMOCOCCAL TYPE 10A SEROTYPE
conditions of pH and Concentration of Polysaccharides,
molecular size, incubation temperature and time.

| | | | Molecular | |
|---|---|---|---|---|
| | | Concentration | size | Incubation time, |
| Condition | pH | of PS | (kDa) | temperature |
| A | PBST (pH 5.5 ± 0.5), 5-6 | 0.5 to 1.0 mg/mL | 990 | 1 Hr/25deg C. |
| B | PBST (pH 6.5 ± 0.5), 6-7 | 0.5 to 1.0 mg/mL | 990 | 1 Hr/25deg C. |
| C | PBST (pH 5.5 ± 0.5), 5-6 | 0.5 to 1.0 mg/mL | 119 | 1 Hr/25deg C. |
| D | PBST (pH 6.5 ± 0.5), 6-7 | 0.5 to 1.0 mg/mL | 119 | 1 Hr/25deg C. |

Inference: Four different conditions (A, B, C, D) were studied to obtain the maximum signal (MFI) of serotype-10A Saccharide coupled microspheres (PS coupled beads). During coupling, the pH range, Saccharide (Ps) Concentration, Molecular size and Incubation time was critically studied. From the above table the maximum MFI signal were observed for condition-D compared with other 3 conditions. Higher the MFI values is directly proportional to the higher sensitivity of the assay, hence parameters mentioned in the condition-D are optimal for coupling serotype-10A antigen to microsphere beads.

TABLE 9
STREPTOCOCCUS PNEUMONIAE/PNEUMOCOCCAL TYPE 24F SEROTYPE
conditions of pH and Concentration of Polysaccharides,
molecular size, incubation temperature and time.

| | | | Molecular | |
|---|---|---|---|---|
| | | Concentration | size | Incubation time, |
| Condition | pH | of PS | (kDa) | temperature |
| A | PBST (pH 5.5 ± 0.5), 4-5 | 0.5 to 1.0 mg/mL | 519 | 1 Hr/25deg C. |
| B | PBST (pH 6.5 ± 0.5), 6-7 | 0.5 to 1.0 mg/mL | 519 | 1 Hr/37deg C. |
| C | PBST (pH 5.5 ± 0.5), 5-6 | 0.5 to 1.0 mg/mL | 81 | 1 Hr/25deg C. |
| D | PBST (pH 6.5 ± 0.5), 6-7 | 0.5 to 1.0 mg/mL | 81 | 1 Hr/25deg C. |

Inference: Four different conditions (A, B, C, D) were studied to obtain the maximum signal (MFI) of serotype-24F Saccharide coupled microsphere beads. During coupling, the pH range, Saccharide (Ps) Concentration, Molecular size and Incubation time was critically studied. From the above table the maximum MFI signal were observed for condition-D compared with other 3 conditions. Higher the MFI values is directly proportional to the higher sensitivity of the assay, hence parameters mentioned in the condition-D are optimal for coupling serotype-24F antigen to microsphere beads.

### Example 7: Streptococcus pneumoniae/Pneumococcal Serotypes 3, 5, 6A, 6B, 9V, 1A, 1A, 12F, 14, 15B, 180, 19A, 19F, 22F, 23F, 24F, 33F

The specific conditions applied to each pneumococcal serotype 3, 5, 6A, 6B, 9V, 10A, 11A, 12F, 14, 15B, 180, 19A, 19F, 22F, 23F, 24F, 33F saccharides during dilution of the saccharide stock and further conjugation to the beads/microspheres gave the optimal MFI's to positive samples.

TABLE 10
STREPTOCOCCUS PNEUMONIAE/PNEUMOCOCCAL SEROTYPES 3, 5, 6A,
6B, 9V, 10A, 11A, 12F, 14, 15B, 18C, 19A, 19F, 22F, 23F, 24F,
33F provides for conditions of pH and Concentration of Polysaccharides,
molecular size, incubation temperature and time.

| | | | PS Conc. | Mol size | |
|---|---|---|---|---|---|
| Serotype | Condition | pH (PBST) | mg/mL | (kDa) | Incubation time |
| 3 | A | (pH 4.5 ± 0.5), 4-5 | 0.5 to 1.0 | 911 | 1 Hr/25° C. |
| 3 | B | (pH 5.5 ± 0.5), 5-6 | 1.0 to 1.5 | 911 | 1 Hr/25° C. |
| 3 | C | (pH 5.5 ± 0.5), 5-6 | 1.5 to 2.0 | 191 | 1 Hr/25° C. |
| 3 | D | (pH 6.5 ± 0.5), 6-7 | 2.0 to 2.5 | 191 | 1 Hr/25° C. |
| 5 | A | (pH 4.5 ± 0.5), 4-5 | 0.5 to 1.0 | 75 | 1 Hr/25° C. |
| 5 | B | (pH 5.5 ± 0.5), 5-6 | 1.0 to 1.5 | 75 | 2 Hr/25° C. |
| 5 | C | (pH 6.5 ± 0.5), 6-7 | 1.5 to 2.0 | 75 | 2 Hr/25° C. |
| 5 | D | (pH 6.5 ± 0.5), 6-7 | 2.0 to 2.5 | 75 | 2 Hr/37° C. |
| 6A | A | (pH 4.5 ± 0.5), 4-5 | 0.5 to 1.0 | 551 | 1 Hr/25° C. |
| 6A | B | (pH 4.5 ± 0.5), 4-5 | 1.0 to 1.5 | 551 | 1.5 Hr/25° C. |
| 6A | C | (pH 4.5 ± 0.5), 4-5 | 1.0 to 1.5 | 551 | 2 Hr/25° C. |
| 6A | D | (pH 4.5 ± 0.5), 4-5 | 1.0 to 1.5 | 511 | 2 Hr/37° C. |
| 6B | A | (pH 4.5 ± 0.5), 4-5 | 0.5 to 1.0 | 706 | 1 Hr/25° C. |
| 6B | B | (pH 4.5 ± 0.5), 4-5 | 0.5 to 1.0 | 706 | 1.5 Hr/25° C. |
| 6B | C | (pH 4.5 ± 0.5), 4-5 | 0.5 to 1.0 | 107 | 1 Hr/25° C. |
| 6B | D | (pH 4.5 ± 0.5), 4-5 | 0.5 to 1.0 | 107 | 1.5 Hr/25° C. |
| 9V | A | (pH 4.5 ± 0.5), 4-5 | 0.5 to 1.0 | 108 | 1 Hr/25° C. |
| 9V | B | (pH 5.5 ± 0.5), 4-5 | 1.0 to 1.5 | 108 | 2 Hr/25° C. |
| 9V | C | (pH 4.5 ± 0.5), 4-5 | 1.0 to 1.5 | 108 | 1 Hr/25° C. |
| 9V | D | (pH 4.5 ± 0.5), 4-5 | 1.0 to 1.5 | 108 | 1.5 Hr/25° C. |
| 10A | A | (pH 4.5 ± 0.5), 4-5 | 0.5 to 1.0 | 119 | 1 Hr/25° C. |
| 10A | B | (pH 5.5 ± 0.5), 5-6 | 0.5 to 1.0 | 119 | 1.5 Hr/25° C. |
| 10A | C | (pH 6.5 ± 0.5), 6-7 | 0.5 to 1.0 | 119 | 1 Hr/25° C. |
| 10A | D | (pH 6.5 ± 0.5), 6-7 | 0.5 to 1.0 | 119 | 1 Hr/25° C. |
| 11A | A | (pH 4.5 ± 0.5), 4-5 | 0.5 to 1.0 | 100 | 1 Hr/25° C. |
| 11A | B | (pH 5.5 ± 0.5), 5-6 | 1.0 to 1.5 | 100 | 1.5 Hr/25° C. |
| 11A | C | (pH 5.5 ± 0.5), 5-6 | 1.5 to 2.0 | 100 | 1.5 Hr/25° C. |
| 11A | D | (pH 5.5 ± 0.5), 5-6 | 2.0 to 2.5 | 100 | 1.5 Hr/25° C. |
| 12F | A | (pH 4.5 ± 0.5), 4-5 | 0.5 to 1.0 | 99 | 1 Hr/25° C. |
| 12F | B | (pH 5.5 ± 0.5), 5-6 | 0.5 to 1.0 | 99 | 1.5 Hr/25° C. |
| 12F | C | (pH 5.5 ± 0.5), 5-6 | 0.5 to 1.0 | 99 | 1.5 Hr/25° C. |
| 12F | D | (pH 6.5 ± 0.5), 6-7 | 0.5 to 1.0 | 99 | 1.5 Hr/25° C. |
| 14 | A | (pH 4.5 ± 0.5), 4-5 | 0.5 to 1.0 | 143 | 1 Hr/25° C. |
| 14 | B | (pH 5.5 ± 0.5), 5-6 | 1.0 to 1.5 | 143 | 1.5 Hr/25° C. |
| 14 | C | (pH 5.5 ± 0.5), 5-6 | 1.5 to 2.0 | 143 | 1.5 Hr/25° C. |
| 14 | D | (pH 6.5 ± 0.5), 6-7 | 2.0 to 2.5 | 143 | 1.5 Hr/25° C. |
| 15B | A | (pH 4.5 ± 0.5), 4-5 | 0.5 to 1.0 | 136 | 1 Hr/25° C. |
| 15B | B | (pH 5.5 ± 0.5), 5-6 | 1.0 to 1.5 | 136 | 1.5 Hr/25° C. |
| 15B | C | (pH 5.5 ± 0.5), 5-6 | 1.5 to 2.0 | 136 | 1.5 Hr/25° C. |
| 15B | D | (pH 5.5 ± 0.5), 5-6 | 2.0 to 2.5 | 136 | 1.5 Hr/25° C. |
| 18C | A | (pH 4.5 ± 0.5), 4-5 | 0.5 to 1.0 | 79 | 1 Hr/25° C. |
| 18C | B | (pH 5.5 ± 0.5), 5-6 | 1.0 to 1.5 | 79 | 1.5 Hr/25° C. |
| 18C | C | (pH 5.5 ± 0.5), 5-6 | 1.5 to 2.0 | 79 | 1.5 Hr/25° C. |
| 18C | D | (pH 6.5 ± 0.5), 6-7 | 2.0 to 2.5 | 79 | 1.5 Hr/25° C. |
| 19A | A | (pH 4.5 ± 0.5), 4-5 | 0.5 to 1.0 | 91 | 1 Hr/25° C. |
| 19A | B | (pH 5.5 ± 0.5), 5-6 | 0.5 to 1.0 | 91 | 1 Hr/37° C. |
| 19A | C | (pH 4.5 ± 0.5), 4-5 | 1.0 to 1.5 | 91 | 1.5 Hr/25° C. |
| 19A | D | (pH 5.5 ± 0.5), 5-6 | 0.5 to 1.0 | 91 | 1.5 Hr/25° C. |
| 19F | A | (pH 4.5 ± 0.5), 4-5 | 0.5 to 1.0 | 583 | 1 Hr/25° C. |
| 19F | B | (pH 4.5 ± 0.5), 4-5 | 0.5 to 1.0 | 583 | 1.5 Hr/25° C. |
| 19F | C | (pH 4.5 ± 0.5), 4-5 | 0.5 to 1.0 | 85 | 1 Hr/25° C. |
| 19F | D | (pH 4.5 ± 0.5), 4-5 | 0.5 to 1.0 | 85 | 1.5 Hr/25° C. |
| 22F | A | (pH 4.5 ± 0.5), 4-5 | 0.5 to 1.0 | 106 | 1 Hr/25° C. |
| 22F | B | (pH 5.5 ± 0.5), 5-6 | 1.0 to 1.5 | 106 | 1 Hr/25° C. |
| 22F | C | (pH 5.5 ± 0.5), 5-6 | 1.5 to 2.0 | 106 | 1 Hr/25° C. |
| 22F | D | (pH 5.5 ± 0.5), 5-6 | 2.0 to 2.5 | 106 | 1 Hr/25° C. |
| 23F | A | (pH 4.5 ± 0.5), 4-5 | 0.5 to 1.0 | 94 | 1 Hr/25° C. |
| 23F | B | (pH 4.5 ± 0.5), 4-5 | 1.0 to 1.5 | 94 | 1 Hr/25° C. |
| 23F | C | (pH 4.5 ± 0.5), 4-5 | 1.5 to 2.0 | 94 | 1 Hr/25° C. |
| 23F | D | (pH 4.5 ± 0.5), 4-5 | 2.0 to 2.5 | 94 | 1 Hr/25° C. |
| 24F | A | (pH 4.5 ± 0.5), 4-5 | 0.5 to 1.0 | 519 | 1 Hr/25° C. |
| 24F | B | (pH 5.5 ± 0.5), 5-6 | 1.0 to 1.5 | 519 | 1 Hr/25° C. |
| 24F | C | (pH 6.5 ± 0.5), 6-7 | 1.5 to 2.0 | 81 | 1 Hr/25° C. |
| 24F | D | (pH 6.5 ± 0.5), 6-7 | 2.0 to 2.5 | 81 | 1 Hr/25° C. |
| 33F | A | (pH 4.5 ± 0.5), 4-5 | 0.5 to 1.0 | 191 | 1 Hr/25° C. |
| 33F | B | (pH 5.5 ± 0.5), 5-6 | 1.0 to 1.5 | 191 | 1 Hr/25° C. |
| 33F | C | (pH 5.5 ± 0.5), 5-6 | 1.5 to 2.0 | 191 | 1 Hr/25° C. |
| 33F | D | (pH 6.5 ± 0.5), 6-7 | 2.0 to 2.5 | 191 | 1 Hr/25° C. |

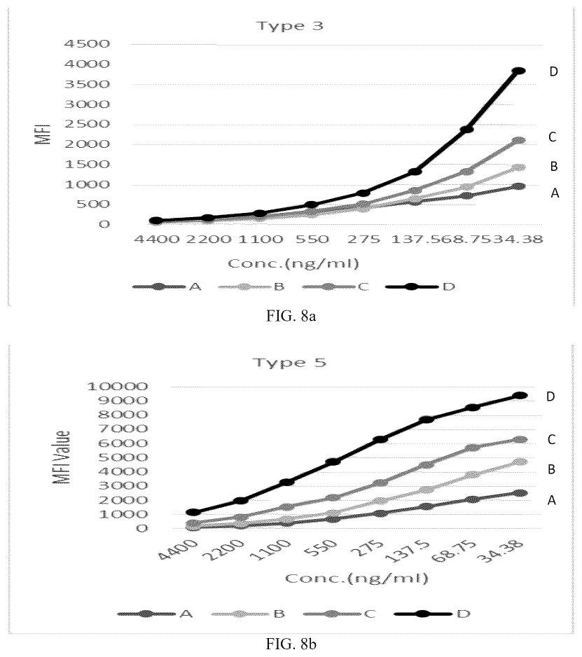

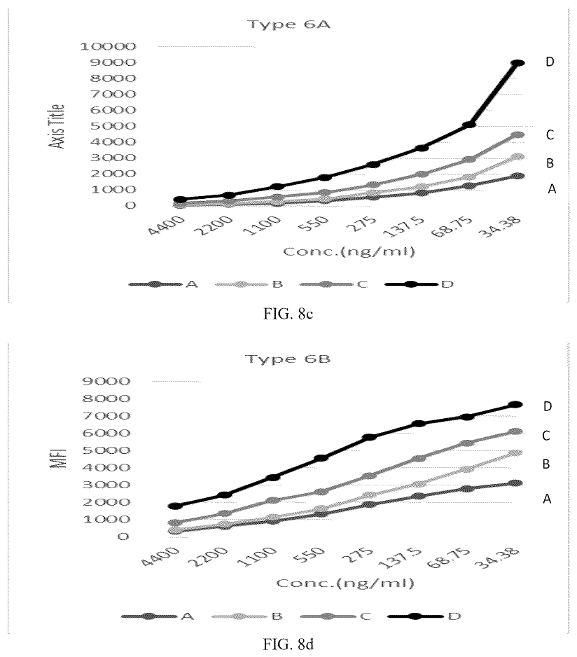

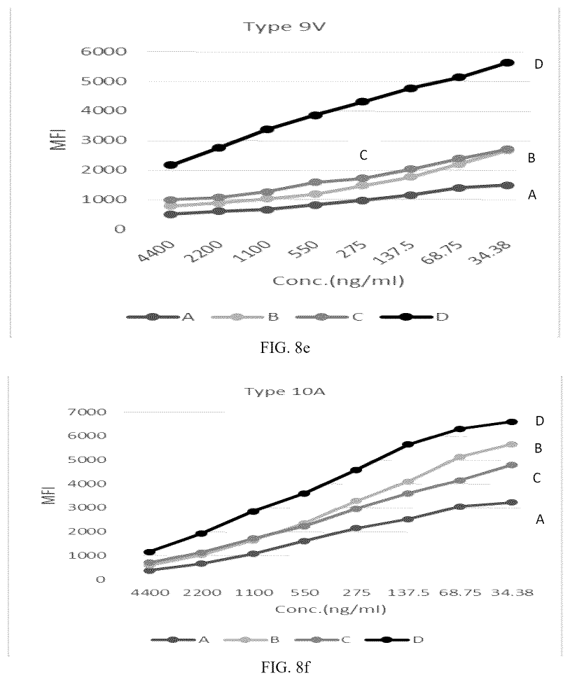

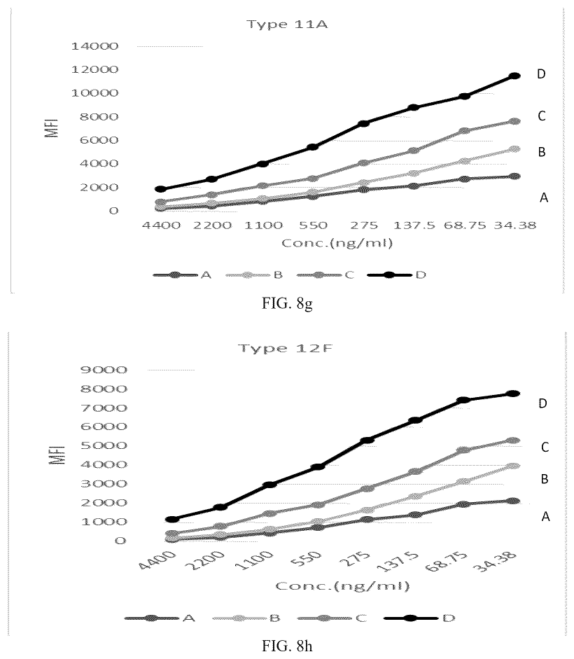

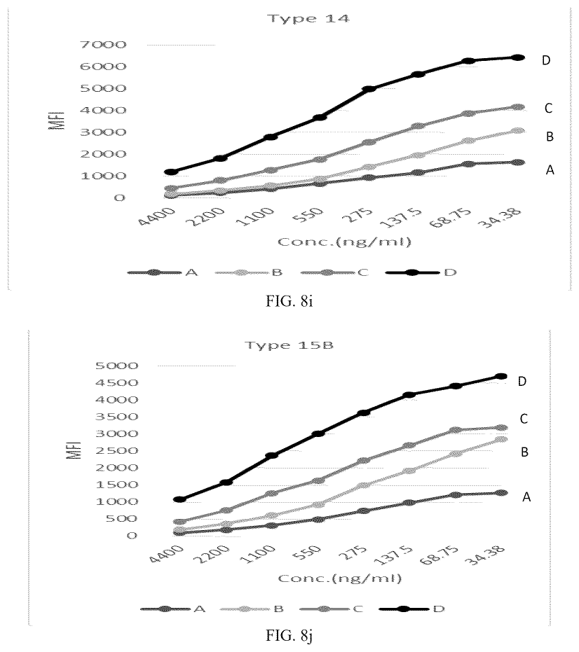

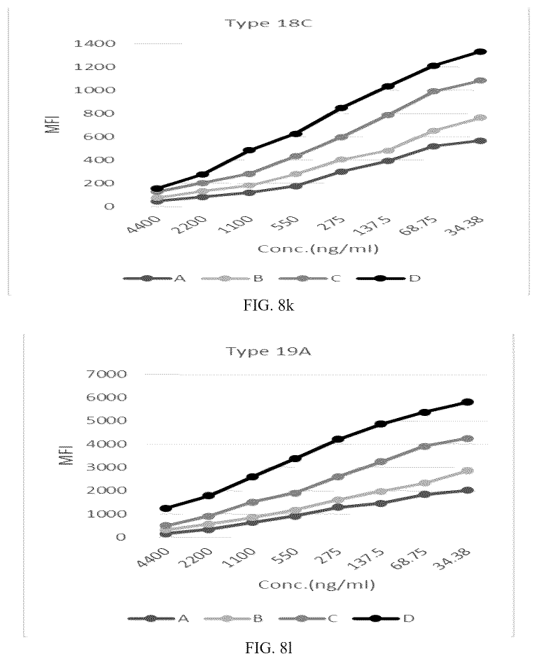

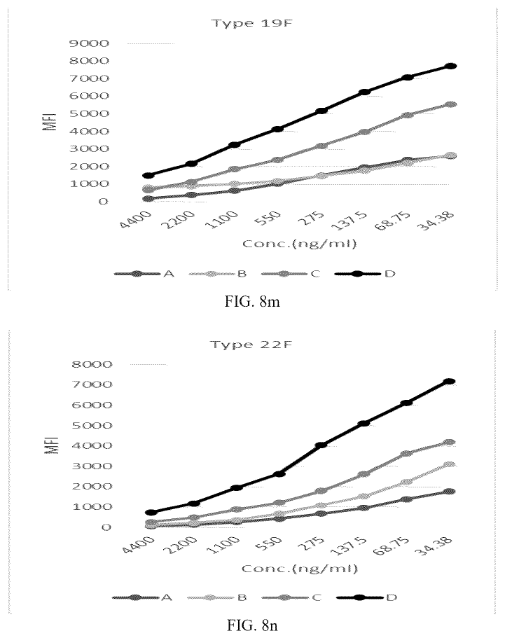

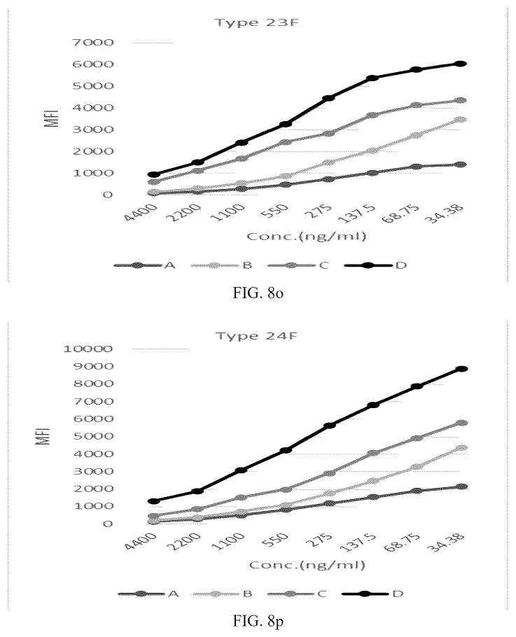

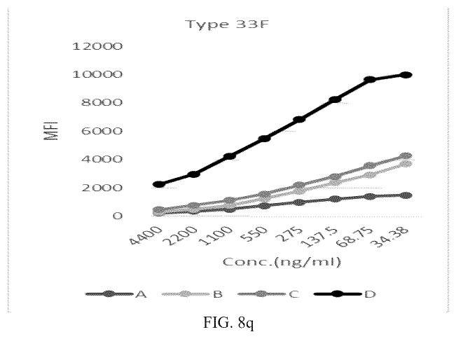

See FIG. 8a to 8q for details of MFI value against pneumococcal serotypes 3, 5, 6A, 6B, 9V, 10A, 11A, 12F, 14, 15B, 18C, 19A, 19F, 22F, 23F, 24F, 33F respectively in conditions of pH and Concentration of Polysaccharides, molecular size, incubation temperature and time.

Inference: Four different conditions (A, B, C, D) were studied to obtain the maximum signal (MFI) of type 3 Saccharide coupled microspheres (PS coupled beads). During coupling the 10 pH range, Saccharide (Ps) Concentration, Molecular size and Incubation time was critically studied. From the above table the maximum MFI signal were observed for condition-D compared with other 3 conditions. If higher the MFI values proportional to the higher sensitivity of the assay, hence parameters mentioned in condition-D is mandatory during coupling of Pneumococcal serotype 3 antigen to microsphere beads.

### Example 8: Haemophilus influenzae Saccharides

The specific conditions applied to Haemophilus influenzae saccharides during dilution of the saccharide stock and further conjugation to the beads/microspheres gave the optimal MFI's to positive samples.

TABLE 11
HAEMOPHILUS INFLUENZAE SACCHARIDES provides
for conditions of pH and Concentration of Polysaccharides,
molecular size (0.3KD for more than 50% of PRP),
incubation temperature and time.

| | | | Incubation time, |
|---|---|---|---|
| Condition | pH of PBST | Ps Conc. (μg/mL) | temperature |
| A | (pH 4.5 ± 0.5), 4-5 | 5 to 10 | 1 Hr/25° C. |
| B | (pH 4.5 ± 0.5), 4-5 | 10 to 15 | 1.5 Hr/25° C. |
| C | (pH 4.5 ± 0.5), 4-5 | 10 to 15 | 2 Hr/25° C. |
| D | (pH 5.5 ± 0.5), 5-6 | 10 to 15 | 1 Hr/25° C. |

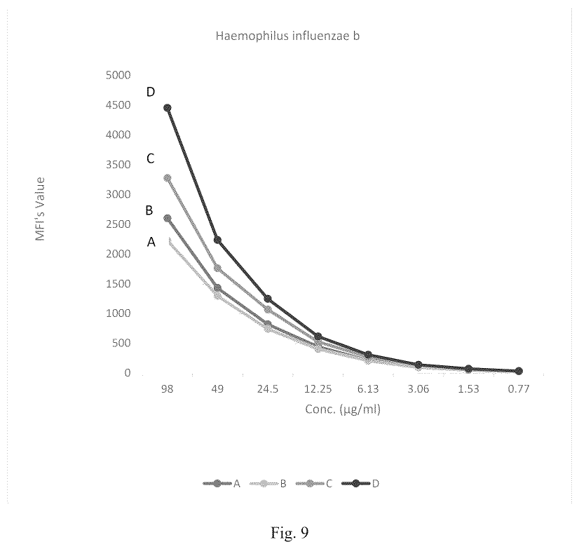

See FIG. 9 for details of MFI value against Haemophilus influenzae saccharides in conditions of pH and Concentration of Polysaccharides, molecular size, incubation temperature and time.

### Example 9: Stability Data for Saccharide Coupled Microsphere

The stability of coupled beads were monitored at two different temperature conditions i.e. 2-8° C. (Real time) and 25° C. (Accelerated). The CV of 30% reduction in the MFI was considered as sign off instability. The data clearly suggests that there is no change in the MFI values throughout the stability the overall % CV was observed <25 indicating the stability of saccharide coupled microspheres using the said process. The stability data for streptococcus serotypes and Haemophilus type b are presented below.

TABLE 12: MFI's (Real time stability at 2° C. to 8° C.) & MFI's (Accelerated at 25° C.) for Pneumococcal Saccharide coupled microspheres

SR=Serotype, ST=Standard, D0/D5/D10/D15=0/5/10/15 Days duration of the stability test, MN=Mean, SD=Standard deviation, % CV=% of coefficient of variation, 1M/3M/6M=1/3/6 Months duration of stability test, BC=Bead Control, S1 to S8=Standards

The assay format was a multiplex in a nature. For antigen content assay format included reference standard, assay blank, Bead control and test sample. Generally, the standard concentration range required for all serotypes except 6B, 4400 ng/ml to 34.38 ng/ml, for 6B 8800 ng/ml to 68.75 ng/ml with serial two-fold dilutions as described below:

Prepared the series of standard from S1 to S8 with serial two fold dilutions as per the following table. Discarded 125 μl from the last well after mixing. Final total volume of each well was 125 μl.

TABLE 12A
STANDARD DILUTIONS

| | | | Concentration (ng/mL) | |
|---|---|---|---|---|
| | | Reagent | Each serotype | serotype |
| Standard | Working standard (μL) | G (μL) | except 6B | 6B |
| S1 | 250 μL working stock | — | 4400 | 8800 |
| S2 | 125 μL of S1 solution | 125 | 2200 | 4400 |
| S3 | 125 μL of S2 solution | 125 | 1100 | 2200 |
| S4 | 125 μL of S3 solution | 125 | 550 | 1100 |
| S5 | 125 μL of S4 solution | 125 | 275 | 550 |
| S6 | 125 μL of S5 solution | 125 | 137.5 | 275 |
| S7 | 125 μL of S6 solution | 125 | 68.75 | 137.5 |
| S8 | 125 μL of S7 solution | 125 | 34.38 | 68.75 |

Bead Control: Bead Control is mixture of all 21 valent coupled beads with their respective antisera. As there is direct binding between antigen and respective antibodies which gives maximum MFI signal compared with S1 to S8. As this is a competitive inhibition assay the antibody mixture first incubated with standard and sample. After incubation, unbound antibody is further incubated with coupled beads. Hence, the signal from S1 to S8 appears increase in order and comparatively less with bead control. Hence, during antibody dilution optimization the dilutions were selected which showed at least minimum of 10% gradation between S8 and bead control to avoid saturation in standard curve formation. Hence, in every assay along with standard bead control value also monitored.

TABLE 12B
Stability data for Pneumococcal Saccharide coupled microspheres for Serotypes 1, 2 and 3.

| | | 1 M | 3 M | 6 M | MN | % CV | D 0 | D 5 | D 10 | D 15 | MN | % CV |
|---|---|---|---|---|---|---|---|---|---|---|---|---|
| SR | ST | Real time stability at 2° C. to 8° C. | | | | | Accelerated at 25° C | | | | | |
| 1 | S1 | 271 | 193 | 201 | 221 | 19 | 216 | 255 | 204 | 211 | 221 | 10 |
| 1 | S2 | 556 | 390 | 405 | 450 | 20 | 409 | 487 | 373 | 391 | 415 | 12 |
| 1 | S3 | 1029 | 660 | 735 | 808 | 24 | 732 | 720 | 725 | 701 | 720 | 2 |
| 1 | S4 | 1503 | 1118 | 1252 | 1291 | 15 | 1228 | 1021 | 1146 | 1165 | 1140 | 8 |
| 1 | S5 | 2287 | 1803 | 2037 | 2042 | 12 | 1716 | 1394 | 1744 | 1679 | 1633 | 10 |
| 1 | S6 | 2950 | 2568 | 2723 | 2747 | 7 | 2408 | 1926 | 2251 | 2335 | 2230 | 10 |
| 1 | S7 | 3840 | 3315 | 3509 | 3554 | 7 | 2950 | 2211 | 2634 | 3109 | 2726 | 15 |
| 1 | S8 | 4765 | 4232 | 4047 | 4348 | 9 | 3578 | 2356 | 3118 | 3635 | 3172 | 19 |
| 1 | BC | — | — | — | — | — | 5411 | 3209 | 4238 | 4602 | 4365 | 21 |
| 2 | S1 | 465 | 439 | 465 | 456 | 3 | 495 | 608 | 550 | 482 | 534 | 11 |
| 2 | S2 | 783 | 740 | 787 | 770 | 3 | 814 | 1073 | 866 | 788 | 885 | 15 |
| 2 | S3 | 1319 | 1216 | 1290 | 1275 | 4 | 1361 | 1668 | 1475 | 1359 | 1466 | 10 |
| 2 | S4 | 2018 | 1822 | 2186 | 2009 | 9 | 2227 | 2490 | 2292 | 2128 | 2284 | 7 |
| 2 | S5 | 3382 | 3126 | 3739 | 3416 | 9 | 3298 | 4208 | 3561 | 3074 | 3535 | 14 |
| 2 | S6 | 5141 | 5188 | 6480 | 5603 | 14 | 5490 | 7349 | 5441 | 4934 | 5804 | 18 |
| 2 | S7 | 9450 | 8761 | 10331 | 9514 | 8 | 8590 | 10223 | 7638 | 8187 | 8659 | 13 |
| 2 | S8 | 13863 | 13631 | 12786 | 13426 | 4 | 11233 | 12867 | 10978 | 11360 | 11609 | 7 |
| 2 | BC | — | — | — | — | — | 13545 | 14293 | 13779 | 13146 | 13691 | 3 |
| 3 | S1 | — | — | — | — | — | 186 | 203 | 191 | 183 | 191 | 4 |
| 3 | S2 | — | — | — | — | — | 252 | 302.3 | 284 | 257 | 264 | 6 |
| 3 | S3 | — | — | — | — | — | 359 | 376 | 426 | 369 | 383 | 8 |
| 3 | S4 | — | — | — | — | — | 502 | 533 | 567 | 566 | 542 | 6 |
| 3 | S5 | — | — | — | — | — | 629 | 672 | 761 | 681 | 686 | 8 |
| 3 | S6 | — | — | — | — | — | 747 | 810 | 865 | 872 | 823 | 7 |
| 3 | S7 | — | — | — | — | — | 871 | 829 | 991 | 966 | 914 | 8 |
| 3 | S8 | — | — | — | — | — | 987 | — | 1158 | 1100 | 1082 | 8 |
| 3 | BC | — | — | — | — | — | 1262 | 1104 | 1345 | 1237 | 1237 | 8 |

The stability study for serotype 1 and 2 coupled beads were performed at real time (2° C. to 8° C.) condition and for serotype 1, 2 and 3 at Accelerated (25° C.) condition. The MFI of reference standard from S1 to S8 were monitored and compiled. The difference in the MFI throughout the stability were calculated in the form of % coefficient of variance (CV) at each standard level. It was observed that serotype 1 and 2 at real time (2° C. to 8° C.) condition and for serotype 1, 2 and 3 at Accelerated (25° C.) condition the % CV were less than 25%. Based on stability study it was concluded that the coupled beads were stable for 6M at real time condition and 15 days at accelerated condition.

TABLE 12C
Stability data for Pneumococcal Saccharide coupled
microspheres for Serotypes 4, 5, 6A, 6B, and 7F.

| | | 1 M | 3 M | 6 M | MN | % CV | D 0 | D 5 | D 10 | D 15 | MN | % CV |
|---|---|---|---|---|---|---|---|---|---|---|---|---|
| SR | ST | Real time stability at 2° C. to 8° C. | | | | | Accelerated at 25° C | | | | | |
| 4 | S1 | 134 | 129 | 144 | 136 | 6 | 162 | 175 | 122 | 135 | 148 | 16 |
| 4 | S2 | 273 | 248 | 277 | 266 | 6 | 273 | 325 | 202 | 221 | 255 | 22 |
| 4 | S3 | 536 | 444 | 550 | 510 | 11 | 471 | 511 | 447 | 445 | 468 | 7 |
| 4 | S4 | 891 | 846 | 1016 | 917 | 10 | 866 | 748 | 752 | 704 | 767 | 9 |
| 4 | S5 | 1515 | 1490 | 1802 | 1602 | 11 | 1270 | 1133 | 1198 | 1137 | 1185 | 5 |
| 4 | S6 | 2239 | 2216 | 2479 | 2311 | 6 | 1965 | 1728 | 1795 | 1776 | 1816 | 6 |
| 4 | S7 | 3092 | 3057 | 3233 | 3127 | 3 | 2461 | 2279 | 2240 | 2467 | 2362 | 5 |
| 4 | S8 | 4103 | 3959 | 3879 | 3980 | 3 | 3316 | 2897 | 2974 | 3197 | 3096 | 6 |
| 4 | BC | — | — | — | — | — | 9661 | 9140 | 8525 | 8364 | 8923 | 7 |
| 5 | S1 | 171 | 142 | 141 | 151 | 11 | 158 | 206 | 138 | 151 | 163 | 18 |
| 5 | S2 | 378 | 298 | 302 | 326 | 14 | 306 | 405 | 268 | 281 | 315 | 20 |
| 5 | S3 | 766 | 553 | 603 | 641 | 17 | 604 | 654 | 563 | 580 | 600 | 7 |
| 5 | S4 | 1241 | 993 | 1133 | 1122 | 11 | 1125 | 1028 | 1021 | 986 | 1040 | 6 |
| 5 | S5 | 2065 | 1768 | 1927 | 1920 | 8 | 1694 | 144 | 1547 | 1586 | 1243 | 59 |
| 5 | S6 | 2781 | 2630 | 2788 | 2733 | 3 | 2516 | 2020 | 2281 | 2404 | 2305 | 9 |
| 5 | S7 | 3710 | 3411 | 3642 | 3588 | 4 | 3149 | 2375 | 2727 | 3094 | 2836 | 13 |
| 5 | S8 | 4741 | 4486 | 4396 | 4541 | 4 | 3882 | 2540 | 3343 | 3918 | 3421 | 19 |
| 5 | BC | — | — | — | — | — | 5886 | 3762 | 4729 | 5141 | 4880 | 18 |
| 6A | S1 | 85 | 75 | 86 | 82 | 7 | 87 | 107 | 90 | 88 | 93 | 10 |
| 6A | S2 | 173 | 138 | 156 | 155 | 11 | 148 | 193 | 153 | 143 | 159 | 14 |
| 6A | S3 | 313 | 248 | 278 | 280 | 12 | 284 | 296 | 293 | 277 | 287 | 3 |
| 6A | S4 | 522 | 415 | 525 | 487 | 13 | 499 | 466 | 535 | 452 | 488 | 8 |
| 6A | S5 | 901 | 780 | 918 | 866 | 9 | 755 | 710 | 861 | 753 | 770 | 8 |
| 6A | S6 | 1332 | 1187 | 1332 | 1284 | 7 | 1161 | 1077 | 1367 | 1118 | 1181 | 11 |
| 6A | S7 | 1968 | 1754 | 2070 | 1931 | 8 | 1627 | 1459 | 1969 | 1665 | 1680 | 13 |
| 6A | S8 | 3789 | 2995 | 3687 | 3490 | 12 | 2626 | 2637 | 3311 | 2633 | 2802 | 12 |
| 6A | BC | — | — | — | — | — | 8610 | 8045 | 7473 | 8364 | 8123 | 6 |
| 6B | S1 | 446 | 361 | 359 | 389 | 13 | 397 | | 406 | 389 | 397 | 2 |
| 6B | S2 | 839 | 643 | 642 | 708 | 16 | 662 | 811 | 667 | 642 | 695 | 11 |
| 6B | S3 | 1318 | 1008 | 1045 | 1124 | 15 | 1065 | 1123 | 1085 | 1098 | 1093 | 2 |
| 6B | S4 | 1867 | 1488 | 1611 | 1655 | 12 | 1604 | 1444 | 1598 | 1607 | 1563 | 5 |
| 6B | S5 | 2613 | 2242 | 2334 | 2396 | 8 | 2132 | 1817 | 2110 | 2093 | 2038 | 7 |
| 6B | S6 | 3214 | 2899 | 2916 | 3010 | 6 | 2708 | 2328 | 2787 | 2649 | 2618 | 8 |
| 6B | S7 | 3975 | 3631 | 3550 | 3718 | 6 | 3187 | 2650 | 2942 | 3186 | 2991 | 9 |
| 6B | S8 | 4470 | 4414 | 3948 | 4277 | 7 | 3631 | | 3461 | 3753 | 3615 | 4 |
| 6B | BC | — | — | — | — | — | 4973 | 3318 | 4174 | 4283 | 4187 | 16 |
| 7F | S1 | 203 | 170 | 155 | 176 | 14 | 150 | 202 | 142 | 149 | 161 | 17 |
| 7F | S2 | 432 | 345 | 317 | 365 | 16 | 291 | 388 | 258 | 267 | 301 | 20 |
| 7F | S3 | 817 | 640 | 597 | 685 | 17 | 534 | 644 | 528 | 507 | 553 | 11 |
| 7F | S4 | 1326 | 1077 | 1074 | 1159 | 12 | 944 | 934 | 906 | 874 | 914 | 3 |
| 7F | S5 | 2181 | 1899 | 1869 | 1983 | 9 | 1449 | 1455 | 1451 | 1334 | 1422 | 4 |
| 7F | S6 | 2863 | 2739 | 2653 | 2752 | 4 | 2241 | 1979 | 2068 | 2032 | 2080 | 5 |
| 7F | S7 | 3836 | 3600 | 3515 | 3650 | 5 | 2811 | 2537 | 2618 | 2759 | 2681 | 5 |
| 7F | S8 | 4990 | 4576 | 4017 | 4527 | 11 | 3342 | 2924 | 3203 | 3570 | 3260 | 8 |
| 7F | BC | — | — | — | — | — | 6376 | 4928 | 5186 | 5430 | 5480 | 12 |

The stability study for serotype 4, 5, 6A, 6B and 7F coupled beads were performed at 2 different conditions i.e. real time (200 to 800) and Accelerated (2500). The MFI of reference standard from S1 to 88 were monitored and compiled. The difference in the MFI throughout the stability were calculated in the form of % coefficient of variance (CV) at each standard level. It was observed that for serotype 4, 5, 6A, 6B and 7F the obtained % CV were less than 25%. Based on stability study it was concluded that the coupled beads were stable for 6M at real time condition and 15 days at accelerated condition.

TABLE 12D
Stability data for Pneumococcal Saccharide coupled microspheres for Serotypes 8, 9V, and 10A.

| | | 1 M | 3 M | 6 M | MN | % CV | D 0 | D 5 | D 10 | D 15 | MN | % CV |
|---|---|---|---|---|---|---|---|---|---|---|---|---|
| SR | ST | Real time stability at 2° C. to 8° C. | | | | | Accelerated at 25° C | | | | | |
| 8 | S1 | 582 | 687 | 678 | 649 | 9 | 811 | 824 | 626 | 688 | 737 | 13 |
| 8 | S2 | 1139 | 1181 | 1202 | 1174 | 3 | 1301 | 1502 | 1070 | 1203 | 1269 | 14 |
| 8 | S3 | 1988 | 1986 | 2060 | 2011 | 2 | 2120 | 2337 | 1981 | 2077 | 2129 | 7 |
| 8 | S4 | 3080 | 2873 | 3684 | 3212 | 13 | 3285 | 3440 | 2916 | 3350 | 3248 | 7 |
| 8 | S5 | 4796 | 4719 | 5401 | 4972 | 8 | 4548 | 4988 | 4200 | 4859 | 4649 | 8 |
| 8 | S6 | 6365 | 6617 | 7666 | 6883 | 10 | 6285 | 7049 | 5447 | 6451 | 6308 | 10 |
| 8 | S7 | 8384 | 8404 | 9207 | 8665 | 5 | 7457 | 7217 | 6067 | 8349 | 7272 | 13 |
| 8 | S8 | 9282 | 10120 | 10103 | 9835 | 5 | 8425 | | 6508 | 9211 | 8048 | 17 |
| 8 | BC | — | — | — | — | — | 11410 | 9537 | 8703 | 10954 | 10151 | 12 |
| 9V | S1 | 752 | 625 | 774 | 717 | 11 | 781 | 1005 | 801 | 857 | 861 | 12 |
| 9V | S2 | 875 | 721 | 917 | 838 | 12 | 929 | 1206 | 875 | 962 | 993 | 15 |
| 9V | S3 | 1072 | 862 | 1044 | 993 | 11 | 1159 | 1325 | 1117 | 1193 | 1198 | 8 |
| 9V | S4 | 1249 | 995 | 1325 | 1189 | 15 | 1485 | 1473 | 1343 | 1399 | 1425 | 5 |
| 9V | S5 | 1577 | 1370 | 1657 | 1535 | 10 | 1629 | 1657 | 1616 | 1638 | 1635 | 1 |
| 9V | S6 | 1836 | 1636 | 1995 | 1822 | 10 | 1999 | 1944 | 1989 | 1952 | 1971 | 1 |
| 9V | S7 | 2246 | 2042 | 2385 | 2224 | 8 | 2205 | 2053 | 2160 | 2377 | 2199 | 6 |
| 9V | S8 | 2578 | 2572 | 2732 | 2627 | 3 | 2490 | 2180 | 2487 | 2641 | 2450 | 8 |
| 9V | BC | — | — | — | — | — | 3214 | 2672 | 2865 | 3051 | 2951 | 8 |
| 10A | S1 | 700 | 679 | 735 | 705 | 4 | 706 | | 801 | 710 | 739 | 7 |
| 10A | S2 | 1201 | 1150 | 1329 | 1226 | 8 | 1118 | 1432 | 1169 | 1197 | 1229 | 11 |
| 10A | S3 | 1831 | 1788 | 2064 | 1894 | 8 | 1811 | 1966 | 1857 | 1938 | 1893 | 4 |
| 10A | S4 | 2649 | 2538 | 3120 | 2769 | 11 | 2775 | 2468 | 2732 | 2693 | 2667 | 5 |
| 10A | S5 | 3556 | 3683 | 4269 | 3836 | 10 | 3448 | 3105 | 3401 | 3612 | 3392 | 6 |
| 10A | S6 | 4346 | 4703 | 5309 | 4786 | 10 | 4404 | 3908 | 4286 | 4477 | 4269 | 6 |
| 10A | S7 | 5214 | 5639 | 5951 | 5601 | 7 | 4711 | 3792 | 4439 | 5362 | 4576 | 14 |
| 10A | S8 | 5691 | 6493 | 6272 | 6152 | 7 | 5486 | 4139 | 4951 | 5741 | 5079 | 14 |
| 10A | BC | — | — | — | — | — | 6532 | 4844 | 5688 | 6006 | 5768 | 12 |

The stability study for serotype 8, 9V, and 10A coupled beads were performed at 2 different conditions i.e. real time (200 to 8° C.) and Accelerated (25a). The MFI of reference standard from 81 to 88 were monitored and compiled. The difference in the MFI throughout the stability were calculated in the form of % coefficient of variance (CV) at each standard level. It was observed that for serotype 8, 9V, and 10A the obtained % CV were less than 25%. Based on stability study it was concluded that the coupled beads were stable for 6M at real time condition and 15 days at accelerated condition.

TABLE 12E
Stability data for Pneumococcal Saccharide coupled microspheres
for Serotypes 11A, 12F, 14, 15B and 18C.

| | | 1 M | 3 M | 6 M | MN | % CV | D 0 | D 5 | D 10 | D 15 | MN | % CV |
|---|---|---|---|---|---|---|---|---|---|---|---|---|
| SR | ST | Real time stability at 2° C. to 8° C. | | | | | Accelerated at 25° C | | | | | |
| 11A | S1 | 409 | 385 | 406 | 400 | 3 | 598 | 717 | 586 | 563 | 616 | 11 |
| 11A | S2 | 759 | 699 | 733 | 730 | 4 | 981 | 1283 | 965 | 941 | 1042 | 15 |
| 11A | S3 | 1313 | 1130 | 1244 | 1229 | 8 | 1605 | 1853 | 1643 | 1615 | 1679 | 7 |
| 11A | S4 | 1870 | 1745 | 2103 | 1906 | 10 | 2506 | 2398 | 2461 | 2411 | 2444 | 2 |
| 11A | S5 | 2663 | 2683 | 3065 | 2804 | 8 | 3389 | 3145 | 3378 | 3312 | 3306 | 3 |
| 11A | S6 | 3522 | 3601 | 4000 | 3707 | 7 | 4589 | 3882 | 4397 | 4454 | 4331 | 7 |
| 11A | S7 | 4396 | 4714 | 5257 | 4789 | 9 | 4937 | 4383 | 5136 | 5433 | 4972 | 9 |
| 11A | S8 | 5808 | 5929 | 6127 | 5955 | 3 | 6148 | 4926 | 5663 | 6499 | 5809 | 12 |
| 11A | BC | — | — | — | — | — | 8188 | 5859 | 7025 | 7178 | 7062 | 14 |
| 12F | S1 | 218 | 205 | 225 | 216 | 5 | 249 | 331 | 244 | 247 | 268 | 16 |
| 12F | S2 | 412 | 395 | 469 | 425 | 9 | 475 | 635 | 468 | 469 | 512 | 16 |
| 12F | S3 | 807 | 711 | 792 | 770 | 7 | 898 | 927 | 854 | 906 | 896 | 3 |
| 12F | S4 | 1285 | 1164 | 1423 | 1291 | 10 | 1495 | 1406 | 1470 | 1499 | 1467 | 3 |
| 12F | S5 | 2038 | 1935 | 2395 | 2123 | 11 | 2135 | 1922 | 2172 | 2077 | 2077 | 5 |
| 12F | S6 | 2588 | 2895 | 3121 | 2868 | 9 | 2988 | 2671 | 3063 | 3046 | 2942 | 6 |
| 12F | S7 | 3650 | 3694 | 4207 | 3850 | 8 | 3825 | 2817 | 3628 | 3887 | 3539 | 14 |
| 12F | S8 | 4387 | 4785 | 4829 | 4667 | 5 | 4560 | 3191 | 4064 | 4717 | 4133 | 17 |
| 12F | BC | — | — | — | — | — | 6328 | 4116 | 5388 | 5387 | 5305 | 17 |
| 14 | S1 | 189 | 168 | 178 | 178 | 6 | 176 | 235 | | 176 | 196 | 17 |
| 14 | S2 | 378 | 323 | 327 | 343 | 9 | 309 | 394 | 255 | 282 | 310 | 19 |
| 14 | S3 | 608 | 538 | 567 | 571 | 6 | 527 | 573 | 465 | 475 | 510 | 10 |
| 14 | S4 | 953 | 851 | 983 | 929 | 7 | 858 | 793 | 721 | 784 | 789 | 7 |
| 14 | S5 | 1491 | 1401 | 1534 | 1475 | 5 | 1181 | 1134 | 1028 | 1069 | 1103 | 6 |
| 14 | S6 | 1900 | 1899 | 1986 | 1928 | 3 | 1661 | 1510 | 1435 | 1630 | 1559 | 7 |
| 14 | S7 | 2464 | 2513 | 2620 | 2533 | 3 | 2010 | 1649 | 1539 | 1935 | 1783 | 13 |
| 14 | S8 | 3039 | 2978 | 2780 | 2932 | 5 | 2318 | 1726 | 1975 | 2294 | 2078 | 14 |
| 14 | BC | — | — | — | — | — | 4178 | 2918 | 3086 | 3259 | 3360 | 17 |
| 15B | S1 | 224 | 215 | 249 | 229 | 8 | 236 | 294 | 234 | 237 | 250 | 12 |
| 15B | S2 | 450 | 425 | 464 | 446 | 4 | 422 | 537 | 391 | 414 | 441 | 15 |
| 15B | S3 | 773 | 674 | 828 | 758 | 10 | 742 | 735 | 740 | 733 | 737 | 1 |
| 15B | S4 | 1137 | 1126 | 1347 | 1204 | 10 | 1127 | 1052 | 1102 | 1089 | 1092 | 3 |
| 15B | S5 | 1667 | 1743 | 1983 | 1798 | 9 | 1546 | 1296 | 1497 | 1529 | 1467 | 8 |
| 15B | S6 | 2153 | 2263 | 2483 | 2300 | 7 | 2115 | 1719 | 1904 | 2015 | 1938 | 9 |
| 15B | S7 | 2618 | 2657 | 2851 | 2709 | 5 | 2474 | 1902 | 2153 | 2509 | 2259 | 13 |
| 15B | S8 | 2954 | 3094 | 3113 | 3054 | 3 | 2707 | 1976 | 2477 | 2868 | 2507 | 16 |
| 15B | BC | — | — | — | — | — | 3538 | 2485 | 2927 | 3231 | 3045 | 15 |
| 18C | S1 | 92 | 74 | 70 | 78 | 15 | 75 | 91 | 74 | 78 | 79 | 10 |
| 18C | S2 | 175 | 128 | 124 | 142 | 20 | 110 | 150 | 109 | 120 | 122 | 16 |
| 18C | S3 | 234 | 183 | 181 | 199 | 15 | 176 | 187 | 192 | 183 | 185 | 4 |
| 18C | S4 | 431 | 284 | 328 | 348 | 22 | 248 | 253 | 283 | 266 | 263 | 6 |
| 18C | S5 | 620 | 388 | 455 | 488 | 25 | 347 | 351 | 404 | 347 | 362 | 8 |
| 18C | S6 | 729 | 593 | 572 | 631 | 14 | 486 | 412 | 206 | 457 | 390 | 32 |
| 18C | S7 | 950 | 677 | 669 | 765 | 21 | 575 | 454 | 566 | 587 | 545 | 11 |
| 18C | S8 | 1063 | 861 | 717 | 881 | 20 | 612 | 497 | 615 | 695 | 605 | 13 |
| 18C | BC | — | — | — | — | — | 1040 | 804 | 1043 | 908 | 949 | 12 |

The stability study for serotype 11A, 12F, 14, 15SB and 180 coupled beads were performed at 2 different conditions i.e. real time (200 to 800) and Accelerated (25° C.). The MFI of reference standard from S1 to 88 were monitored and compiled. The difference in the MFI throughout the stability were calculated in the form of % coefficient of variance (CV) at each standard level. It was observed that for serotype 11A, 12F, 14, 15B and 180 the obtained % CV were less than 25%. Based on stability study it was concluded that the coupled beads were stable for 6M at real time condition and 15 days at accelerated condition.

TABLE 12F
Stability data for Pneumococcal Saccharide coupled
microspheres for Serotypes 19A, 19F, and 22F.

| | | 1 M | 3 M | 6 M | MN | | D 0 | D 5 | D 10 | D 15 | MN | |
|---|---|---|---|---|---|---|---|---|---|---|---|---|
| SR | ST | Real time stability at 2° C. to 8° C. | | | | % CV | Accelerated at 25° C | | | | | % CV |
| 19A | S1 | 267 | 231 | 245 | 248 | 7 | 242 | 310 | 235 | 233 | 255 | 14 |
| 19A | S2 | 520 | 452 | 453 | 475 | 8 | 426 | 539 | 421 | 431 | 454 | 12 |
| 19A | S3 | 922 | 741 | 779 | 814 | 12 | 743 | 778 | 764 | 770 | 764 | 2 |
| 19A | S4 | 1253 | 1149 | 1234 | 1212 | 5 | 1159 | 1063 | 1112 | 1156 | 1123 | 4 |
| 19A | S5 | 1883 | 1725 | 1846 | 1818 | 5 | 1617 | 1410 | 1532 | 1566 | 1531 | 6 |
| 19A | S6 | 2251 | 2197 | 2265 | 2238 | 2 | 2111 | 1757 | 2039 | 1952 | 1965 | 8 |
| 19A | S7 | 2927 | 2731 | 2761 | 2806 | 4 | 2402 | 1878 | 2227 | 2690 | 2299 | 15 |
| 19A | S8 | 3359 | 3295 | 3081 | 3245 | 4 | 2742 | 2062 | 2609 | 2898 | 2578 | 14 |
| 19A | BC | — | — | — | — | — | 4039 | 2559 | 3301 | 3409 | 3327 | 18 |
| 19F | S1 | 337 | 283 | 305 | 308 | 9 | 317 | 402 | 313 | 305 | 334 | 14 |
| 19F | S2 | 628 | 539 | 583 | 583 | 8 | 566 | 709 | 543 | 568 | 596 | 13 |
| 19F | S3 | 1138 | 896 | 1020 | 1018 | 12 | 966 | 1025 | 1003 | 990 | 996 | 2 |
| 19F | S4 | 1573 | 1362 | 1620 | 1518 | 9 | 1534 | 1382 | 1560 | 1498 | 1493 | 5 |
| 19F | S5 | 2276 | 2011 | 2419 | 2235 | 9 | 2055 | 1813 | 2113 | 2161 | 2035 | 8 |
| 19F | S6 | 2830 | 2808 | 3000 | 2879 | 4 | 2829 | 2372 | 2674 | 2684 | 2640 | 7 |
| 19F | S7 | 3644 | 3438 | 2860 | 3314 | 12 | 3226 | 2679 | 3166 | 3537 | 3152 | 11 |
| 19F | S8 | 4544 | 4389 | 4359 | 4431 | 2 | 3795 | 2793 | 3747 | 3982 | 3579 | 15 |
| 19F | BC | — | — | — | — | — | 7523 | 4839 | 5191 | 6084 | 5909 | 20 |
| 22F | S1 | 134 | 134 | 158 | 142 | 9 | 222 | 274 | 184 | 203 | 221 | 18 |
| 22F | S2 | 282 | 251 | 297 | 276 | 8 | 366 | 505 | 359 | 358 | 397 | 18 |
| 22F | S3 | 479 | 461 | 585 | 508 | 13 | 690 | 751 | 679 | 646 | 692 | 6 |
| 22F | S4 | 796 | 774 | 1037 | 869 | 17 | 1209 | 1111 | 1103 | 1071 | 1124 | 5 |
| 22F | S5 | 1371 | 1323 | 1655 | 1450 | 12 | 1666 | 1517 | 1598 | 1664 | 1611 | 4 |
| 22F | S6 | 1827 | 1967 | 2351 | 2048 | 13 | 2376 | 2159 | 2544 | 2245 | 2331 | 7 |
| 22F | S7 | 2578 | 2680 | 3264 | 2840 | 13 | 3073 | 2616 | 2737 | 3030 | 2864 | 8 |
| 22F | S8 | 3706 | 3651 | 4485 | 3948 | 12 | 3628 | 3042 | 3585 | 3851 | 3527 | 10 |
| 22F | BC | — | — | — | — | — | 9291 | 6159 | 8809 | 7595 | 7963 | 18 |

The stability study for serotype 19A, 19F, and 22F coupled beads were performed at 2 different conditions i.e. real time (200 to 800) and Accelerated (2500). The MFI of reference standard from 81 to 88 were monitored and compiled. The difference in the MFI throughout the stability were calculated in the form of % coefficient of variance (CV) at each standard level. It was observed that for serotype 19A, 19F, and 22F the obtained % CV were less than 25%. Based on stability study it was concluded that the coupled beads were stable for 6M at real time condition and 15 days at accelerated condition

TABLE 12G
Stability data for Pneumococcal Saccharide coupled
microspheres for Serotypes 23F, 24F, and 33F.

| | | 1 M | 3 M | 6 M | MN | | D 0 | D 5 | D 10 | D 15 | MN | |
|---|---|---|---|---|---|---|---|---|---|---|---|---|
| SR | ST | Real time stability at 2° C. to 8° C. | | | | % CV | Accelerated at 25° C | | | | | % CV |
| 23F | S1 | 161 | 133 | 130 | 141 | 12 | 135 | 169 | 118 | 130 | 138 | 16 |
| 23F | S2 | 328 | 258 | 259 | 282 | 14 | 237 | 326 | 226 | 234 | 256 | 18 |
| 23F | S3 | 658 | 486 | 503 | 549 | 17 | 470 | 495 | 448 | 463 | 469 | 4 |
| 23F | S4 | 1013 | 807 | 917 | 912 | 11 | 848 | 746 | 797 | 757 | 787 | 6 |
| 23F | S5 | 1605 | 1468 | 1529 | 1534 | 4 | 1222 | 997 | 1158 | 1166 | 1136 | 9 |
| 23F | S6 | 2105 | 2007 | 2085 | 2066 | 3 | 1822 | 1331 | 1564 | 1780 | 1624 | 14 |
| 23F | S7 | 2661 | 2679 | 2517 | 2619 | 3 | 2179 | 1643 | 1909 | 2240 | 1993 | 14 |
| 23F | S8 | 3534 | 3349 | 3056 | 3313 | 7 | 2654 | 1748 | 2431 | 2800 | 2408 | 19 |
| 23F | BC | — | — | — | — | — | 4023 | 2530 | 3164 | 3443 | 3290 | 19 |
| 24F | S1 | 249 | 225 | 290 | 255 | 13 | 374 | 467 | 356 | 365 | 390 | 13 |
| 24F | S2 | 475 | 483 | 515 | 491 | 4 | 674 | 851 | 609 | 642 | 694 | 16 |
| 24F | S3 | 951 | 774 | 951 | 892 | 11 | 1229 | 1281 | 1202 | 1160 | 1218 | 4 |
| 24F | S4 | 1342 | 1208 | 1696 | 1415 | 18 | 1938 | 1764 | 1841 | 1833 | 1844 | 4 |
| 24F | S5 | 2048 | 2113 | 2567 | 2243 | 13 | 2564 | 2412 | 2681 | 2514 | 2543 | 4 |
| 24F | S6 | 2729 | 2943 | 3647 | 3106 | 15 | 3726 | 3301 | 3604 | 3611 | 3560 | 5 |
| 24F | S7 | 3746 | 3909 | 4672 | 4109 | 12 | 4236 | 3566 | 4156 | 4696 | 4164 | 11 |
| 24F | S8 | 4631 | 5195 | 5459 | 5095 | 8 | 5268 | 3807 | 5127 | 5460 | 4915 | 15 |
| 24F | BC | — | — | — | — | — | 7672 | 5423 | 6736 | 6782 | 6653 | 14 |
| 33F | S1 | 517 | 517 | 555 | 530 | 4 | 752 | 936 | 753 | 740 | 795 | 12 |
| 33F | S2 | 849 | 889 | 953 | 897 | 6 | 1173 | 1479 | 1133 | 1131 | 1229 | 14 |
| 33F | S3 | 1339 | 1296 | 1384 | 1339 | 3 | 1768 | 1992 | 1832 | 1819 | 1853 | 5 |
| 33F | S4 | 1795 | 1775 | 2176 | 1915 | 12 | 2467 | 2468 | 2473 | 2541 | 2487 | 1 |
| 33F | S5 | 2565 | 2524 | 3018 | 2702 | 10 | 3073 | 3137 | 3102 | 3326 | 3159 | 4 |
| 33F | S6 | 3078 | 3453 | 3952 | 3494 | 13 | 4178 | 3875 | 4093 | 4062 | 4052 | 3 |
| 33F | S7 | 4088 | 4228 | 5034 | 4450 | 11 | 4767 | 4242 | 4595 | 5150 | 4689 | 8 |
| 33F | S8 | 4987 | 5551 | 5610 | 5383 | 6 | 5341 | 4761 | 5303 | 5873 | 5319 | 9 |
| 33F | BC | — | — | — | — | — | 7201 | 5464 | 6455 | 6582 | 6425 | 11 |

The stability study for serotype 23F, 24F, and 33F coupled beads were performed at 2 different conditions i.e. real time (200 to 800) and Accelerated (2500). The MFI of reference standard from S1 to 88 were monitored and compiled. The difference in the MFI throughout the stability were calculated in the form of % coefficient of variance (CV) at each standard level. It was observed that for serotype 23F, 24F, and 33F the obtained % CV were less than 25%. Based on stability study it was concluded that the coupled beads were stable for 6M at real time condition and 15 days at accelerated condition

TABLE 12H
Stability data for Haemophilus influenzae.

| | 1 M | 3 M | 8 M | Mean | % CV | Initial | 1 M | 3 M | Mean | % CV |
|---|---|---|---|---|---|---|---|---|---|---|
| STD | Real time stability at 2° C. to 8° C. | | | | | Accelerated at 25° C | | | | |
| S1 | 4627 | 4458 | 4366 | 4554 | 4 | 4609 | 4891 | 4458 | 4653 | 5 |
| S2 | 2502 | 2456 | 2365 | 2376 | 6 | 2645 | 3047 | 2456 | 2716 | 11 |
| S3 | 1255 | 1254 | 1090 | 1140 | 12 | 1418 | 1525 | 1254 | 1399 | 10 |
| S4 | 673 | 672 | 514 | 574 | 21 | 698 | 737 | 672 | 702 | 5 |
| S5 | 315 | 334 | 245 | 284 | 17 | 344 | 403 | 334 | 360 | 10 |
| S6 | 152 | 172 | 126 | 141 | 18 | 169 | 199 | 172 | 180 | 9 |
| S7 | 62 | 82 | 57 | 65 | 17 | 77 | 102 | 82 | 87 | 15 |
| S8 | 31 | 37 | 31 | 30 | 22 | 36 | 69 | 37 | 47 | 40 |

The stability study for Hib PRP coupled beads were performed at 2 different conditions i.e. real time (200 to 8° C.) and Accelerated (2500). The MFI of reference standard from S to 8 were monitored and compiled. The difference in the MFI throughout the stability were calculated in the form of % coefficient of variance (CV) at each standard level. It was observed that for Hib PRP the obtained % CV were less than 25%. Based on stability study it was concluded that the coupled beads were stable for 8M at real time condition and 3M at accelerated condition

### Example 10: Antigenic Integrity of Saccharide Coupled Microsphere

The antigenic integrity of the saccharide coupled microspheres/antigens were assessed using serological reactivity with specific antibodies generated in the different species such as mice, rat, rabbit and human. The antigenic integrity was confirmed by using following approaches:
a) No cross reactions on beads: The negative sera value was near to baseline.
b) Dilutional linearity with positive sera: The dilution linearity was determined by monitoring the % gradation between each dilution. The acceptable percentage gradation during each dilution should be more than 10%.

Below table indicate that the saccharide coupled microspheres showed no cross reactions as all the MFI's with negative sera MFI value was <100. Also, in the case of positive sera the beads showed good dilution linearity from 1:100 to 1:102400 dilution supporting that the epitopes of each antigen were unaffected during coupling process.

### Table 13 (Table 13A to 13E): Antigenic Integrity of Saccharide Coupled Microspheres of Pneumococcal Polysaccharides of Serotypes 1, 2, 4, 5, 6A, 6B, 7F, 8, 9V, 10A, 11A, 12F, 14, 15B, 18C, 19A, 19F, 22F, 23F, 24F, 33F.

Table 13 (A to E) provides for the method evaluating immunogenicity of immunogenic composition where the saccharide coupled microsphere corresponding to the saccharide in the immunogenic composition are used.

TABLE 13A
For Pneumococcal Serotypes 1, 2 and 4

| | Parameter | | | | | Gradation (%) between | | | |
|---|---|---|---|---|---|---|---|---|---|
| | Species | MFI | | | | each dilution | | | |
| SR | sera | Mice | Rabbit | Rat | Human | Mice | Rabbit | Rat | Human |
| 1 | NS | 16 | 16 | 17 | 4 | — | — | — | — |
| 1 | 1:100 | 9559 | 10999 | 13358 | 15400 | — | — | — | — |
| 1 | 1:200 | 6699 | 8261 | 10069 | 6298 | 30 | 25 | 25 | 59 |
| 1 | 1:400 | 4869 | 6101 | 7587 | 4307 | 27 | 26 | 25 | 32 |
| 1 | 1:800 | 2693 | 5178 | 5269 | 1770 | 45 | 15 | 31 | 59 |
| 1 | 1:1600 | 1410 | 3692 | 3920 | 1261 | 48 | 29 | 26 | 29 |
| 1 | 1:3200 | 824 | 2707 | 2949 | 527 | 42 | 27 | 25 | 58 |
| 1 | 1:6400 | 388 | 1857 | 1984 | 297 | 53 | 31 | 33 | 44 |
| 1 | 1:12800 | 217 | 1262 | 1648 | 156 | 44 | 32 | 17 | 47 |
| 1 | 1:25600 | 126 | 816 | 1122 | 98 | 42 | 35 | 32 | 37 |
| 1 | 1:51200 | 69 | 406 | 537 | 67 | 46 | 50 | 52 | 32 |
| 1 | 1:102400 | 43 | 185 | 230 | 24 | 37 | 54 | 57 | 64 |
| 2 | NS | 8 | 13 | 14 | 4 | — | — | — | — |
| 2 | 100 | 2049 | 22383 | 21062 | 27326 | — | — | — | — |
| 2 | 200 | 1334 | 17894 | 16273 | 24308 | 35 | 20 | 23 | 11 |
| 2 | 400 | 973 | 13339 | 11940 | 20756 | 27 | 25 | 27 | 15 |
| 2 | 800 | 610 | 10087 | 7594 | 9674 | 37 | 24 | 36 | 53 |
| 2 | 1600 | 360 | 7127 | 5764 | 6372 | 41 | 29 | 24 | 34 |
| 2 | 3200 | 232 | 5298 | 4353 | 2975 | 36 | 26 | 24 | 53 |
| 2 | 6400 | 108 | 3855 | 3263 | 1553 | 54 | 27 | 25 | 48 |
| 2 | 12800 | 60 | 2768 | 2879 | 945 | 44 | 28 | 12 | 39 |
| 2 | 25600 | 35 | 1965 | 2110 | 536 | 42 | 29 | 27 | 43 |
| 2 | 51200 | 21 | 1122 | 1223 | 338 | 40 | 43 | 42 | 37 |
| 2 | 102400 | 12 | 575 | 544 | 155 | 43 | 49 | 56 | 54 |
| 4 | NS | 16 | 13 | 13 | 9 | — | — | — | — |
| 4 | 100 | 6837 | 15247 | 12260 | 12997 | — | — | — | — |
| 4 | 200 | 5144 | 10205 | 7186 | 10034 | 25 | 33 | 41 | 23 |
| 4 | 400 | 3863 | 6439 | 4056 | 5830 | 25 | 37 | 44 | 42 |
| 4 | 800 | 2345 | 4157 | 2162 | 2798 | 39 | 35 | 47 | 52 |
| 4 | 1600 | 1291 | 2179 | 1149 | 1779 | 45 | 48 | 47 | 36 |
| 4 | 3200 | 784 | 1259 | 557 | 958 | 39 | 42 | 52 | 46 |
| 4 | 6400 | 357 | 604 | 287 | 486 | 55 | 52 | 49 | 49 |
| 4 | 12800 | 184 | 288 | 183 | 266 | 48 | 52 | 36 | 45 |
| 4 | 25600 | 104 | 140 | 108 | 149 | 44 | 52 | 41 | 44 |
| 4 | 51200 | 54 | 62 | 48 | 66 | 48 | 56 | 56 | 56 |
| 4 | 102400 | 33 | 35 | 28 | 30 | 39 | 44 | 42 | 55 |

SR = Serotype,
NS = Negative Sera

TABLE 13B
For Pneumococcal Serotypes 5, 6A, 6B and 7F

| SR | Species sera | Mice | Rabbit | Rat | Human | Mice | Rabbit | Rat | Human |
|---|---|---|---|---|---|---|---|---|---|
| 5 | NS | 14 | 10 | 11 | 9 | — | — | — | — |
| 5 | 100 | 7572 | 17177 | 19337 | 21039 | — | — | — | — |
| 5 | 200 | 5207 | 12610 | 14506 | 10728 | 31 | 27 | 25 | 49 |
| 5 | 400 | 3625 | 9453 | 10663 | 6758 | 30 | 25 | 26 | 37 |
| 5 | 800 | 1940 | 7222 | 7054 | 3099 | 46 | 24 | 34 | 54 |
| 5 | 1600 | 851 | 5259 | 5263 | 1972 | 56 | 27 | 25 | 36 |
| 5 | 3200 | 557 | 3744 | 3556 | 822 | 35 | 29 | 32 | 58 |
| 5 | 6400 | 212 | 22911 | 2250 | 457 | 62 | −512 | 37 | 44 |
| 5 | 12800 | 124 | 1461 | 1813 | 262 | 42 | 94 | 19 | 43 |
| 5 | 25600 | 71 | 817 | 1112 | 140 | 43 | 44 | 39 | 47 |
| 5 | 51200 | 38 | 331 | 479 | 87 | 46 | 59 | 57 | 38 |
| 5 | 102400 | 26 | 145 | 166 | 39 | 33 | 56 | 65 | 55 |
| 6A | NS | 10 | 18 | 18 | 10 | — | — | — | — |
| 6A | 100 | 2806 | 21802 | 11160 | 9670 | — | — | — | — |
| 6A | 200 | 1783 | 18371 | 8727 | 7650 | 36 | 16 | 22 | 21 |
| 6A | 400 | 1206 | 16173 | 6085 | 4581 | 32 | 12 | 30 | 40 |
| 6A | 800 | 611 | 14049 | 3899 | 2179 | 49 | 13 | 36 | 52 |
| 6A | 1600 | 282 | 9915 | 2628 | 1359 | 54 | 29 | 33 | 38 |
| 6A | 3200 | 164 | 6735 | 1622 | 704 | 42 | 32 | 38 | 48 |
| 6A | 6400 | 68 | 4117 | 971 | 322 | 59 | 39 | 40 | 54 |
| 6A | 12800 | 40 | 2643 | 757 | 179 | 41 | 36 | 22 | 44 |
| 6A | 25600 | 25 | 1659 | 43 | 94 | 38 | 37 | 43 | 47 |
| 6A | 51200 | 16 | 920 | 222 | 44 | 36 | 45 | 48 | 53 |
| 6A | 102400 | 13 | 502 | 93 | 21 | 19 | 45 | 58 | 52 |
| 6B | NS | 11 | 47 | 47 | 11 | — | — | — | — |
| 6B | 100 | 5074 | 13258 | 13559 | 18222 | — | — | — | — |
| 6B | 200 | 3223 | 9423 | 10185 | 8454 | 36 | 29 | 25 | 54 |
| 6B | 400 | 2263 | 7070 | 8070 | 7197 | 30 | 25 | 21 | 15 |
| 6B | 800 | 1263 | 5900 | 6030 | 3050 | 44 | 17 | 25 | 58 |
| 6B | 1600 | 573 | 4397 | 4716 | 2014 | 55 | 25 | 22 | 34 |
| 6B | 3200 | 412 | 3505 | 3494 | 832 | 28 | 20 | 26 | 59 |
| 6B | 6400 | 171 | 2655 | 2706 | 360 | 58 | 24 | 23 | 57 |
| 6B | 12800 | 95 | 1931 | 2229 | 262 | 44 | 27 | 18 | 27 |
| 6B | 25600 | 55 | 1309 | 1619 | 127 | 42 | 32 | 27 | 52 |
| 6B | 51200 | 32 | 816 | 986 | 62 | 42 | 38 | 39 | 51 |
| 6B | 102400 | 23 | 460 | 460 | 34 | 28 | 44 | 53 | 45 |
| 7F | NS | 15 | 15 | 15 | 17 | — | — | — | — |
| 7F | 100 | 9813 | 17952 | 20970 | 13051 | — | — | — | — |
| 7F | 200 | 8882 | 14193 | 17660 | 9777 | 9 | 21 | 16 | 25 |
| 7F | 400 | 7636 | 10034 | 13987 | 5364 | 14 | 29 | 21 | 45 |
| 7F | 800 | 5304 | 7120 | 9688 | 2443 | 31 | 29 | 31 | 54 |
| 7F | 1600 | 3634 | 4539 | 6488 | 1528 | 31 | 36 | 33 | 37 |
| 7F | 3200 | 2629 | 2821 | 3960 | 807 | 28 | 38 | 39 | 47 |
| 7F | 6400 | 1510 | 1503 | 2094 | 417 | 43 | 47 | 47 | 48 |
| 7F | 12800 | 905 | 769 | 1486 | 224 | 40 | 49 | 29 | 46 |
| 7F | 25600 | 481 | 399 | 828 | 112 | 47 | 48 | 44 | 50 |
| 7F | 51200 | 257 | 142 | 368 | 49 | 47 | 64 | 56 | 56 |
| 7F | 102400 | 126 | 71 | 152 | 21 | 51 | 50 | 59 | 57 |

TABLE 13C
For Pneumococcal Serotypes 8, 9V, 10A, 11A

| SR | Species sera | Mice | Rabbit | Rat | Human | Mice | Rabbit | Rat | Human |
|---|---|---|---|---|---|---|---|---|---|
| 8 | NS | 9 | 20 | 20 | 17 | — | — | — | — |
| 8 | 100 | 1350 | 23847 | 23946 | 14182 | — | — | — | — |
| 8 | 200 | 999 | 22621 | 22291 | 11238 | 26 | 5 | 7 | 21 |
| 8 | 400 | 553 | 17901 | 16970 | 6607 | 45 | 21 | 24 | 41 |
| 8 | 800 | 254 | 13322 | 11816 | 3161 | 54 | 26 | 30 | 52 |
| 8 | 1600 | 119 | 9761 | 8145 | 1987 | 53 | 27 | 31 | 37 |
| 8 | 3200 | 78 | 6621 | 5614 | 1071 | 35 | 32 | 31 | 46 |
| 8 | 6400 | 34 | 4685 | 3841 | 550 | 56 | 29 | 32 | 49 |
| 8 | 12800 | 23 | 2899 | 3045 | 312 | 32 | 38 | 21 | 43 |
| 8 | 25600 | 15 | 2118 | 2018 | 166 | 35 | 27 | 34 | 47 |
| 8 | 51200 | 12 | 1019 | 1143 | 78 | 20 | 52 | 43 | 53 |
| 8 | 102400 | 12 | 578 | 509 | 35 | 0 | 43 | 55 | 55 |
| 9V | NS | 14 | 19 | 19 | 6 | — | — | — | — |
| 9V | 100 | 5335 | 15518 | 14491 | 26041 | — | — | — | — |
| 9V | 200 | 4348 | 12626 | 10955 | 23384 | 19 | 19 | 24 | 10 |
| 9V | 400 | 3503 | 10827 | 8405 | 15373 | 19 | 14 | 23 | 34 |
| 9V | 800 | 2306 | 8237 | 5675 | 6962 | 34 | 24 | 32 | 55 |
| 9V | 1600 | 1442 | 5803 | 4439 | 4462 | 37 | 30 | 22 | 36 |
| 9V | 3200 | 934 | 3957 | 3035 | 2036 | 35 | 32 | 32 | 54 |
| 9V | 6400 | 408 | 2742 | 2245 | 1148 | 56 | 31 | 26 | 44 |
| 9V | 12800 | 242 | 2001 | 1965 | 642 | 41 | 27 | 12 | 44 |
| 9V | 25600 | 130 | 1388 | 1429 | 358 | 46 | 31 | 27 | 44 |
| 9V | 51200 | 60 | 775 | 810 | 236 | 54 | 44 | 43 | 34 |
| 9V | 102400 | 34 | 427 | 383 | 108 | 43 | 45 | 53 | 54 |
| 10A | NS | 9 | 18 | 19 | 18 | — | — | — | — |
| 10A | 100 | 6294 | 22246 | 21230 | 13271 | — | — | — | — |
| 10A | 200 | 4233 | 16623 | 16084 | 10479 | 33 | 25 | 24 | 21 |
| 10A | 400 | 3141 | 12411 | 10682 | 6016 | 26 | 25 | 34 | 43 |
| 10A | 800 | 1745 | 9075 | 6590 | 2986 | 44 | 27 | 38 | 50 |
| 10A | 1600 | 871 | 5349 | 4212 | 1858 | 50 | 41 | 36 | 38 |
| 10A | 3200 | 537 | 3402 | 2263 | 999 | 38 | 36 | 46 | 46 |
| 10A | 6400 | 210 | 1846 | 1159 | 486 | 61 | 46 | 49 | 51 |
| 10A | 12800 | 114 | 976 | 829 | 265 | 46 | 47 | 28 | 45 |
| 10A | 25600 | 59 | 527 | 416 | 145 | 48 | 46 | 50 | 45 |
| 10A | 51200 | 35 | 222 | 181 | 68 | 41 | 58 | 56 | 53 |
| 10A | 102400 | 18 | 118 | 80 | 32 | 49 | 47 | 56 | 53 |
| 11A | NS | 15 | 18 | 18 | 17 | — | — | — | — |
| 11A | 100 | 11656 | 23861 | 23923 | 15049 | — | — | — | — |
| 11A | 200 | 10694 | 20808 | 23523 | 8637 | 8 | 13 | 2 | 43 |
| 11A | 400 | 8985 | 17201 | 19529 | 4839 | 16 | 17 | 17 | 44 |
| 11A | 800 | 6334 | 14756 | 14438 | 2978 | 30 | 14 | 26 | 38 |
| 11A | 1600 | 4211 | 10865 | 12536 | 1620 | 34 | 26 | 13 | 46 |
| 11A | 3200 | 3321 | 8914 | 8982 | 883 | 21 | 18 | 28 | 45 |
| 11A | 6400 | 1713 | 6648 | 6766 | 527 | 48 | 25 | 25 | 40 |
| 11A | 12800 | 1123 | 5094 | 6338 | 258 | 34 | 23 | 6 | 51 |
| 11A | 25600 | 580 | 3817 | 4471 | 91 | 48 | 25 | 29 | 65 |
| 11A | 51200 | 307 | 2114 | 2729 | 62 | 47 | 45 | 39 | 32 |
| 11A | 102400 | 157 | 1276 | 1317 | 31 | 49 | 40 | 52 | 50 |

TABLE 13D
For Pneumococcal Serotypes 12F, 14, 15B, 18C,

| SR | Species sera | Mice | Rabbit | Rat | Human | Mice | Rabbit | Rat | Human |
|---|---|---|---|---|---|---|---|---|---|
| 12F | NS | 4 | 15 | 14 | 2 | — | — | — | — |
| 12F | 100 | 4387 | 15695 | 17720 | 13709 | — | — | — | — |
| 12F | 200 | 2391 | 13958 | 12997 | 8509 | 45 | 11 | 27 | 38 |
| 12F | 400 | 1726 | 10942 | 9886 | 4812 | 28 | 22 | 24 | 43 |
| 12F | 800 | 753 | 9136 | 7213 | 2862 | 56 | 17 | 27 | 41 |
| 12F | 1600 | 352 | 6842 | 5795 | 1543 | 53 | 25 | 20 | 46 |
| 12F | 3200 | 216 | 4898 | 4508 | 786 | 39 | 28 | 22 | 49 |
| 12F | 6400 | 75 | 3550 | 3191 | 500 | 65 | 28 | 29 | 36 |
| 12F | 12800 | 53 | 2232 | 2581 | 225 | 30 | 37 | 19 | 55 |
| 12F | 25600 | 25 | 1392 | 1700 | 80 | 52 | 38 | 34 | 64 |
| 12F | 51200 | 17 | 648 | 878 | 57 | 32 | 53 | 48 | 29 |
| 12F | 102400 | 9 | 303 | 349 | 28 | 47 | 53 | 60 | 51 |
| 14 | NS | 15 | 16 | 17 | 14 | — | — | — | — |
| 14 | 100 | 22490 | 23201 | 24261 | 24924 | — | — | — | — |
| 14 | 200 | 19266 | 18259 | 21247 | 21508 | 14 | 21 | 12 | 14 |
| 14 | 400 | 21518 | 13229 | 15104 | 14928 | −12 | 28 | 29 | 31 |
| 14 | 800 | 19386 | 9119 | 7829 | 6695 | 10 | 31 | 48 | 55 |
| 14 | 1600 | 15626 | 5708 | 4651 | 4257 | 19 | 37 | 41 | 36 |
| 14 | 3200 | 12974 | 3041 | 1986 | 2018 | 17 | 47 | 57 | 53 |
| 14 | 6400 | 7865 | 1764 | 985 | 1113 | 39 | 42 | 50 | 45 |
| 14 | 12800 | 6079 | 949 | 677 | 626 | 23 | 46 | 31 | 44 |
| 14 | 25600 | 4050 | 504 | 329 | 348 | 33 | 47 | 51 | 44 |
| 14 | 51200 | 2247 | 221 | 146 | 214 | 45 | 56 | 56 | 39 |
| 14 | 102400 | 1304 | 110 | 63 | 99 | 42 | 50 | 57 | 54 |
| 15B | NS | 9 | 18 | 19 | 3 | — | — | — | — |
| 15B | 100 | 3256 | 12046 | 11743 | 18097 | — | — | — | — |
| 15B | 200 | 2203 | 9147 | 8356 | 11524 | 32 | 24 | 29 | 36 |
| 15B | 400 | 1590 | 6288 | 5491 | 7094 | 28 | 31 | 34 | 38 |
| 15B | 800 | 948 | 4697 | 3061 | 4109 | 40 | 25 | 44 | 42 |
| 15B | 1600 | 515 | 2755 | 1712 | 2279 | 46 | 41 | 44 | 45 |
| 15B | 3200 | 357 | 1464 | 947 | 1127 | 31 | 47 | 45 | 51 |
| 15B | 6400 | 166 | 859 | 476 | 683 | 54 | 41 | 50 | 39 |
| 15B | 12800 | 98 | 454 | 325 | 362 | 41 | 47 | 32 | 47 |
| 15B | 25600 | 56 | 245 | 195 | 135 | 43 | 46 | 40 | 63 |
| 15B | 51200 | 31 | 115 | 95 | 86 | 45 | 53 | 52 | 36 |
| 15B | 102400 | 19 | 63 | 52 | 42 | 39 | 45 | 45 | 51 |
| 18C | NS | 10 | 19 | 19 | 7 | — | — | — | — |
| 18C | 100 | 3268 | 3287 | 3458 | 25369 | — | — | — | — |
| 18C | 200 | 2235 | 2193 | 2119 | 23669 | 32 | 33 | 39 | 7 |
| 18C | 400 | 1740 | 1580 | 1233 | 15624 | 22 | 28 | 42 | 34 |
| 18C | 800 | 1301 | 1114 | 720 | 9211 | 25 | 29 | 42 | 41 |
| 18C | 1600 | 826 | 509 | 471 | 4920 | 36 | 54 | 35 | 47 |
| 18C | 3200 | 634 | 323 | 271 | 2492 | 23 | 36 | 42 | 49 |
| 18C | 6400 | 354 | 194 | 164 | 1458 | 44 | 40 | 39 | 41 |
| 18C | 12800 | 221 | 112 | 137 | 702 | 38 | 42 | 16 | 52 |
| 18C | 25600 | 124 | 76 | 91 | 303 | 44 | 32 | 34 | 57 |
| 18C | 51200 | 72 | 43 | 52 | 214 | 42 | 43 | 43 | 29 |
| 18C | 102400 | 42 | 30 | 29 | 126 | 42 | 30 | 44 | 41 |

TABLE 13E
For Pneumococcal Serotypes 19A, 19F, 22F, 23F, 24F, 33F

| SR | Species sera | Mice | Rabbit | Rat | Human | Mice | Rabbit | Rat | Human |
|---|---|---|---|---|---|---|---|---|---|
| 19A | NS | 14 | 14 | 15 | 15 | — | — | — | — |
| 19A | 100 | 9262 | 16223 | 15051 | 25021 | — | — | — | — |
| 19A | 200 | 7402 | 12199 | 11870 | 22824 | 20 | 25 | 21 | 9 |
| 19A | 400 | 6617 | 10296 | 8851 | 16862 | 11 | 16 | 25 | 26 |
| 19A | 800 | 4932 | 8488 | 5883 | 8017 | 25 | 18 | 34 | 52 |
| 19A | 1600 | 3212 | 5552 | 4126 | 5346 | 35 | 35 | 30 | 33 |
| 19A | 3200 | 2380 | 3550 | 2638 | 2393 | 26 | 36 | 36 | 55 |
| 19A | 6400 | 1176 | 2384 | 1635 | 1309 | 51 | 33 | 38 | 45 |
| 19A | 12800 | 728 | 1496 | 1221 | 776 | 38 | 37 | 25 | 41 |
| 19A | 25600 | 380 | 915 | 853 | 449 | 48 | 39 | 30 | 42 |
| 19A | 51200 | 189 | 441 | 408 | 292 | 50 | 52 | 52 | 35 |
| 19A | 102400 | 86 | 199 | 178 | 127 | 54 | 55 | 56 | 57 |
| 19F | NS | 9 | 17 | 19 | 22 | — | — | — | — |
| 19F | 100 | 8436 | 17089 | 18148 | 16622 | — | — | — | — |
| 19F | 200 | 5605 | 13338 | 13470 | 6150 | 34 | 22 | 26 | 63 |
| 19F | 400 | 4072 | 8954 | 9763 | 4410 | 27 | 33 | 28 | 28 |
| 19F | 800 | 2157 | 7582 | 6068 | 1913 | 47 | 15 | 38 | 57 |
| 19F | 1600 | 1137 | 4781 | 4330 | 1212 | 47 | 37 | 29 | 37 |
| 19F | 3200 | 710 | 3485 | 3270 | 566 | 38 | 27 | 24 | 53 |
| 19F | 6400 | 293 | 2382 | 225 | 309 | 59 | 32 | 93 | 45 |
| 19F | 12800 | 178 | 1554 | 1838 | 144 | 39 | 35 | −717 | 53 |
| 19F | 25600 | 83 | 929 | 1170 | 86 | 53 | 40 | 36 | 40 |
| 19F | 51200 | 49 | 434 | 597 | 47 | 42 | 53 | 49 | 45 |
| 19F | 102400 | 24 | 211 | 228 | 21 | 51 | 51 | 62 | 55 |
| 22F | NS | 16 | 15 | 17 | 7 | — | — | — | — |
| 22F | 100 | 13123 | 22741 | 23765 | 23136 | — | — | — | — |
| 22F | 200 | 11800 | 20543 | 21698 | 13704 | 10 | 10 | 9 | 41 |
| 22F | 400 | 11177 | 16673 | 18897 | 8473 | 5 | 19 | 13 | 38 |
| 22F | 800 | 10090 | 12869 | 15837 | 5090 | 10 | 23 | 16 | 40 |
| 22F | 1600 | 8626 | 9881 | 13785 | 2644 | 15 | 23 | 13 | 48 |
| 22F | 3200 | 7449 | 7124 | 11245 | 1308 | 14 | 28 | 18 | 51 |
| 22F | 6400 | 5466 | 4845 | 7399 | 816 | 27 | 32 | 34 | 38 |
| 22F | 12800 | 3694 | 2875 | 6312 | 420 | 32 | 41 | 15 | 49 |
| 22F | 25600 | 2657 | 2183 | 4163 | 136 | 28 | 24 | 34 | 68 |
| 22F | 51200 | 1566 | 978 | 2033 | 90 | 41 | 55 | 51 | 34 |
| 22F | 102400 | 998 | 535 | 978 | 47 | 36 | 45 | 52 | 48 |
| 23F | NS | 14 | 16 | 15 | 14 | — | — | — | — |
| 23F | 100 | 3221 | 14898 | 16421 | 15723 | — | — | — | — |
| 23F | 200 | 2000 | 10997 | 13505 | 7198 | 38 | 26 | 18 | 54 |
| 23F | 400 | 1203 | 8337 | 9590 | 4339 | 40 | 24 | 29 | 40 |
| 23F | 800 | 520 | 6058 | 6288 | 2123 | 57 | 27 | 34 | 51 |
| 23F | 1600 | 238 | 3803 | 3972 | 1297 | 54 | 37 | 37 | 39 |
| 23F | 3200 | 154 | 2366 | 2501 | 674 | 35 | 38 | 37 | 48 |
| 23F | 6400 | 67 | 1364 | 1414 | 309 | 56 | 42 | 43 | 54 |
| 23F | 12800 | 47 | 807 | 986 | 176 | 31 | 41 | 30 | 43 |
| 23F | 25600 | 32 | 460 | 574 | 92 | 31 | 43 | 42 | 48 |
| 23F | 51200 | 23 | 158 | 250 | 54 | 28 | 66 | 57 | 41 |
| 23F | 102400 | 17 | 79 | 95 | 22 | 26 | 50 | 62 | 59 |
| 24F | NS | 10 | 21 | 19 | 16 | — | — | — | — |
| 24F | 100 | 5029 | 23586 | 23536 | 9782 | — | — | — | — |
| 24F | 200 | 3051 | 22799 | 19700 | 7529 | 39 | 3 | 16 | 23 |
| 24F | 400 | 2064 | 18496 | 15186 | 4269 | 32 | 19 | 23 | 43 |
| 24F | 800 | 950 | 14444 | 11404 | 2063 | 54 | 22 | 25 | 52 |
| 24F | 1600 | 491 | 11401 | 9372 | 1308 | 48 | 21 | 18 | 37 |
| 24F | 3200 | 284 | 9081 | 7561 | 694 | 42 | 20 | 19 | 47 |
| 24F | 6400 | 112 | 6672 | 5833 | 340 | 61 | 27 | 23 | 51 |
| 24F | 12800 | 77 | 4876 | 5063 | 182 | 31 | 27 | 13 | 46 |
| 24F | 25600 | 40 | 3426 | 3859 | 95 | 48 | 30 | 24 | 48 |
| 24F | 51200 | 27 | 2094 | 2292 | 45 | 33 | 39 | 41 | 53 |
| 24F | 102400 | 17 | 1216 | 1252 | 20 | 37 | 42 | 45 | 56 |
| 33F | NS | 9 | 20 | 21 | 48 | — | — | — | — |
| 33F | 100 | 3760 | 21128 | 22505 | 22398 | — | — | — | — |
| 33F | 200 | 2171 | 17035 | 16919 | 15045 | 42 | 19 | 25 | 33 |
| 33F | 400 | 1281 | 13558 | 13855 | 9537 | 41 | 20 | 18 | 37 |
| 33F | 800 | 537 | 10987 | 10337 | 6267 | 58 | 19 | 25 | 34 |
| 33F | 1600 | 249 | 9244 | 8356 | 3327 | 54 | 16 | 19 | 47 |
| 33F | 3200 | 170 | 7151 | 6977 | 1820 | 32 | 23 | 17 | 45 |
| 33F | 6400 | 78 | 5365 | 5181 | 1139 | 54 | 25 | 26 | 37 |
| 33F | 12800 | 43 | 3921 | 4265 | 537 | 45 | 27 | 18 | 53 |
| 33F | 25600 | 31 | 2550 | 2913 | 212 | 28 | 35 | 32 | 61 |
| 33F | 51200 | 20 | 1353 | 1647 | 119 | 35 | 47 | 43 | 44 |
| 33F | 102400 | 14 | 674 | 690 | 59 | 30 | 50 | 58 | 50 |

### Example 11: Comparison of Conventional Method of Coupling and Method of Present Invention

Below is comparison of the present method of coupling compared with the conventional method of coupling, i.e. Two step carbodiimide reaction.

TABLE 14
CONVENTIONAL AND PRESENT METHOD OF COUPLING
MICROSPHERE WITH SACCHARIDE

| Parameters | Conventional coupling Chemistries | Method of Present Invention |
|---|---|---|
| Concentration of | 2 mg | Variable based on Bacterial |
| Saccharides | | serotypes, for e.g. 0.5-1 mg/ml, |
| | | 1.5-2.0 mg/ml and 2.0-2.5 mg/ml |
| | | serotype-wise. |
| Size of the | Native Saccharides | Requires specific Saccharides |
| Saccharides | | sizes to allow electro-static |
| | | interactions |
| Principle of the | Two step carbodiimide reaction | Utilises metal polymer |
| method | which involves chemical | complexes binding of the target |
| | modification PnPS were | molecule through chelating to the |
| | conjugated to poly-l-lysine or | electron donating groups of the |
| | oxidation chemistry | ligand to be coupled. No |
| | | modification of Saccharides |
| Conditions require | The antigens (protein) to be | Antigen (polysaccharide) were |
| for Bead Coupling | coupled were prepared using | diluted using different PBST |
| | conjugation buffer. | buffers with different pH as listed |
| | Protein molecule: 50 to 100 | in tables listed herein for different |
| | μg/ml), conjugation buffer pH 5.2 | Bacterial saccharides and |
| | | different serotypes of same |
| | | bacteria |
| Incubation time | Antigen (protein) were mixed | Antigens (polysaccharide) were |
| and temperature | with the activated beads and | mixed with the activated beads |
| | incubated at room temperature | and incubated at different |
| | for 60 min | temperature as listed in tables |
| | | listed herein for different |
| | | Bacterial saccharides and |
| | | different serotypes of same |
| | | bacteria |
| Blocking Agent | 2.0% BSA | 1.0% BSA |
| Cross linking agent | Required | Not required |
| (Cyanuric chloride) | | |
| Toxic/hygroscopic | Yes | No toxic materials used |
| chemicals | | |
| Freshly prepared | Requires fresh reagents | Not required |
| reagents | | |
| Bead Scale-up | Up to 4 mL | Up to 100 mL |
| Time duration for | 15-20 days | 1-2 days |
| coupling | | |
| Time duration for | 7-10 days | 2-4 days |
| Qualification | | |
| Microsphere | Non-Magnetic/magnetic | Non-Magnetic/Magnetic |

### Example 12: Performance of Saccharide Coupled Microspheres

The performance of the saccharide coupled microspheres of present disclosure was compared with saccharide coupled microspheres formed using known reagents and the result is summarized in Table 15. The performance was compared by calculating the % difference values between MFI's. The saccharide coupled microspheres using the method of present disclosure showed higher MFI's (more than 50%) for all the serotypes at each dilution level. The MFI difference with the method of present disclosure lead to higher sensitivity in the assay.

The comparison of the data indicates the unaffected epitopes during coupling by the two processes.

Inference: The performance is compared by calculating the % difference values between MFI's. The microsphere/bead coupled with the saccharides using the method as disclosed herein showed higher MFI's (more than 50%) for all the microsphere coupled with saccharides of serotypes at each dilution level. This MFI difference with the said process will lead to higher sensitivity in the assay.

TABLE 15
SACCHARIDE COUPLED MICROSPHERE PERFORMANCE

| SR | R | B | 100 | 200 | 400 | 800 | 1600 | 3200 | 6400 | 12800 | 25600 | 51200 | 102400 |
|---|---|---|---|---|---|---|---|---|---|---|---|---|---|
| 1 | P | 4 | 15400 | 6298 | 4307 | 1770 | 1261 | 527 | 297 | 156 | 98 | 67 | 24 |
| 1 | I | 19 | 3430 | 2567 | 1455 | 598 | 441 | 190 | 87 | 52 | 45 | 29 | 22 |
| 1 | D | | 78 | 59 | 66 | 66 | 65 | 64 | 71 | 67 | 54 | 57 | 8 |
| 2 | P | 4 | 27326 | 24308 | 20756 | 9674 | 6372 | 2975 | 1553 | 945 | 536 | 338 | 155 |
| 2 | I | 8 | 13506 | 10792 | 6133 | 2736 | 1742 | 942 | 480 | 279 | 156 | 72 | 34 |
| 2 | D | | 51 | 56 | 70 | 72 | 73 | 68 | 69 | 70 | 71 | 79 | 78 |
| 4 | P | 9 | 12997 | 10034 | 5830 | 2798 | 1779 | 958 | 486 | 266 | 149 | 66 | 30 |
| 4 | I | 20 | 7740 | 4892 | 3221 | 2030 | 1340 | 595 | 584 | 287 | 73 | 59 | 35 |
| 4 | D | | 40 | 51 | 45 | 27 | 25 | 38 | −20 | −8 | 51 | 11 | −17 |
| 5 | P | 9 | 21039 | 10728 | 6758 | 3099 | 1972 | 822 | 457 | 262 | 140 | 87 | 39 |
| 5 | I | 19 | 10164 | 7942 | 4710 | 2221 | 1404 | 731 | 355 | 192 | 103 | 46 | 23 |
| 5 | D | | 52 | 26 | 30 | 28 | 29 | 11 | 22 | 27 | 26 | 47 | 41 |
| 6A | P | 10 | 9670 | 7650 | 4581 | 2179 | 1359 | 704 | 322 | 179 | 94 | 44 | 21 |
| 6A | I | 20 | 6111 | 2717 | 1758 | 794 | 545 | 188 | 96 | 60 | 24 | 14 | 4 |
| 6A | D | | 37 | 64 | 62 | 64 | 60 | 73 | 70 | 66 | 74 | 68 | 81 |
| 6B | P | 11 | 18222 | 8454 | 7197 | 3050 | 2014 | 832 | 360 | 262 | 127 | 62 | 34 |
| 6B | I | 10 | 8935 | 6908 | 3951 | 1946 | 1239 | 666 | 345 | 188 | 100 | 46 | 21 |
| 6B | D | | 51 | 18 | 45 | 36 | 38 | 20 | 4 | 28 | 21 | 26 | 38 |
| 7F | P | 17 | 13051 | 9777 | 5364 | 2443 | 1528 | 807 | 417 | 224 | 112 | 49 | 21 |
| 7F | I | 14 | 4797 | 2886 | 1686 | 835 | 490 | 262 | 107 | 63 | 47 | 28 | 14 |
| 7F | D | | 63 | 70 | 69 | 66 | 68 | 68 | 74 | 72 | 58 | 43 | 33 |
| 8 | P | 17 | 14182 | 11238 | 6607 | 3161 | 1987 | 1071 | 550 | 312 | 166 | 78 | 35 |
| 8 | I | 9 | 4433 | 2399 | 2871 | 1737 | 675 | 422 | 260 | 102 | 49 | 34 | 20 |
| 8 | D | | 69 | 79 | 57 | 45 | 66 | 61 | 53 | 67 | 70 | 56 | 43 |
| 9V | P | 6 | 26041 | 23384 | 15373 | 6962 | 4462 | 2036 | 1148 | 642 | 358 | 236 | 108 |
| 9V | I | 9 | 16514 | 13147 | 7709 | 3768 | 2370 | 1293 | 661 | 367 | 200 | 96 | 42 |
| 9V | D | | 37 | 44 | 50 | 46 | 47 | 36 | 42 | 43 | 44 | 59 | 61 |
| 10A | P | 18 | 13271 | 10479 | 6016 | 2986 | 1858 | 999 | 486 | 265 | 145 | 68 | 32 |
| 10A | I | 7 | 7436 | 5413 | 3254 | 2149 | 936 | 669 | 494 | 181 | 67 | 42 | 24 |
| 10A | D | | 44 | 48 | 46 | 28 | 50 | 33 | −2 | 32 | 54 | 38 | 25 |
| 11A | P | 17 | 15049 | 8637 | 4839 | 2978 | 1620 | 883 | 527 | 258 | 91 | 62 | 31 |
| 11A | I | 13 | 13438 | 10518 | 6121 | 2995 | 1867 | 1010 | 512 | 287 | 159 | 78 | 35 |
| 11A | D | | 11 | −22 | −26 | −1 | −15 | −14 | 3 | −11 | −75 | −26 | −13 |
| 12F | P | 2 | 13709 | 8509 | 4812 | 2862 | 1543 | 786 | 500 | 225 | 80 | 57 | 28 |
| 12F | I | 3 | 7941 | 5922 | 3372 | 1561 | 983 | 508 | 237 | 129 | 71 | 32 | 15 |
| 12F | D | | 42 | 30 | 30 | 45 | 36 | 35 | 53 | 43 | 11 | 44 | 46 |
| 14 | P | 14 | 24924 | 21508 | 14928 | 6695 | 4257 | 2018 | 1113 | 626 | 348 | 214 | 99 |
| 14 | I | 9 | 24373 | 23767 | 13975 | 6370 | 3921 | 2077 | 1059 | 609 | 342 | 165 | 75 |
| 14 | D | | 2 | −11 | 6 | 5 | 8 | −3 | 5 | 3 | 2 | 23 | 24 |
| 15B | P | 3 | 18097 | 11524 | 7094 | 4109 | 2279 | 1127 | 683 | 362 | 135 | 86 | 42 |
| 15B | I | 8 | 8262 | 6484 | 3812 | 1824 | 1173 | 602 | 301 | 162 | 86 | 40 | 18 |
| 15B | D | | 54 | 44 | 46 | 56 | 49 | 47 | 56 | 55 | 36 | 53 | 57 |
| 18C | P | 7 | 25369 | 23669 | 15624 | 9211 | 4920 | 2492 | 1458 | 702 | 303 | 214 | 126 |
| 18C | I | 17 | 12371 | 9859 | 5979 | 3017 | 1889 | 1042 | 529 | 307 | 166 | 79 | 39 |
| 18C | D | | 51 | 58 | 62 | 67 | 62 | 58 | 64 | 56 | 45 | 63 | 69 |
| 19A | P | 15 | 25021 | 22824 | 16862 | 8017 | 5346 | 2393 | 1309 | 776 | 449 | 292 | 127 |
| 19A | I | 9 | 10537 | 9121 | 6608 | 3886 | 2654 | 1507 | 845 | 497 | 293 | 146 | 69 |
| 19A | D | | 58 | 60 | 61 | 52 | 50 | 37 | 35 | 36 | 35 | 50 | 46 |
| 19F | P | 22 | 16622 | 6150 | 4410 | 1913 | 1212 | 566 | 309 | 144 | 86 | 47 | 21 |
| 19F | I | 11 | 5904 | 4695 | 2826 | 1428 | 908 | 502 | 254 | 134 | 69 | 32 | 15 |
| 19F | D | | 64 | 24 | 36 | 25 | 25 | 11 | 18 | 7 | 20 | 32 | 29 |
| 22F | P | 7 | 23136 | 13704 | 8473 | 5090 | 2644 | 1308 | 816 | 420 | 136 | 90 | 47 |
| 22F | I | 14 | 13862 | 10878 | 6051 | 2909 | 1851 | 958 | 488 | 264 | 145 | 67 | 31 |
| 22F | D | | 40 | 21 | 29 | 43 | 30 | 27 | 40 | 37 | −7 | 26 | 34 |
| 23F | P | 14 | 15723 | 7198 | 4339 | 2123 | 1297 | 674 | 309 | 176 | 92 | 54 | 22 |
| 23F | I | 11 | 10202 | 7846 | 4361 | 2072 | 1286 | 662 | 310 | 171 | 91 | 43 | 19 |
| 23F | D | | 35 | −9 | −1 | 2 | 1 | 2 | 0 | 3 | 1 | 20 | 14 |
| 24F | P | 16 | 9782 | 7529 | 4269 | 2063 | 1308 | 694 | 340 | 182 | 95 | 45 | 20 |
| 24F | I | 34 | 4974 | 3049 | 2286 | 1457 | 795 | 441 | 274 | 157 | 56 | 32 | 29 |
| 24F | D | | 49 | 60 | 46 | 29 | 39 | 36 | 19 | 14 | 41 | 29 | −45 |
| 33F | P | 48 | 22398 | 15045 | 9537 | 6267 | 3327 | 1820 | 1139 | 537 | 212 | 119 | 59 |
| 33F | I | 11 | 17645 | 13648 | 7891 | 3783 | 2372 | 1293 | 659 | 363 | 199 | 90 | 41 |
| 33F | D | | 21 | 9 | 17 | 40 | 29 | 29 | 42 | 32 | 6 | 24 | 31 |

SR = Serotypes,
B = Blank,
R = Dilution reference,
P = Prior art,
I = Applicants invention saccharide coupled microsphere,
D = Percentage (%) Difference

### Example 13: Coupling Ratio of the Polysaccharide to Bead in Formation of Saccharide Coupled Microspheres

The specific ratios and assay conditions associated with the saccharide coupled microspheres is provided below.

The coupling ratio is dependent on the requirement of saccharide concentration and blank microsphere/beads per 100 μL. The ratio is calculated as follows:

Coupling ratio=No. of microspheres (Beads) (00 μl)/Concentration of Saccharides (PS) required for coupling (100 μl).

The significance of maximum ratio indicates requirement of less concentration of saccharide for coupling.

TABLE 16
COUPLING RATIOS OF SACCHARIDES AND MICROSPHERES

| | Desired polysaccharide | | | | | | | | |
|---|---|---|---|---|---|---|---|---|---|
| | conditions | | | Desired bead | | | | Bead recovery | |
| | | | (PBST) | conditions | | | | after coupling | |
| | | Size | Dilution of | Bead | | | PS to | No. of | |
| | Conc. | of Ps | PnPS | stock | | | bead | Beads | Recovery |
| Type | (μg) | (kDa) | stocks | solution | BN | BL | ratio | per μl | (%) |
| 1 | 50 to | 80 | (pH 5.5 ± | 100 μl | 1.25 | 12500 | 125- | 10000 | 80 |
| | 100 | | 0.5), 5-6 | beads | | | 250 | | |
| 2 | 150 to | 1011 | (pH 4.5 ± | 100 μl | 1.25 | 12500 | 62.5- | 10200 | 82 |
| | 200 | | 0.5), 4-5 | beads | | | 83.3 | | |
| 4 | 50 to | 87 | (pH 6.5 ± | 100 μl | 1.25 | 12500 | 125- | 9940 | 80 |
| | 100 | | 0.5), 6-7 | beads | | | 250 | | |
| 5 | 200 to | 75 | (pH 6.5 ± | 100 μl | 1.25 | 12500 | 50- | 10500 | 84 |
| | 250 | | 0.5), 6-7 | beads | | | 62.5 | | |
| 6A | 150 to | 551 | (pH 4.5 ± | 100 μl | 1.25 | 12500 | 62.5- | 9940 | 80 |
| | 200 | | 0.5), 4-5 | beads | | | 83.3 | | |
| 6B | 50 to | 107 | (pH 5.5 ± | 100 μl | 1.25 | 12500 | 125- | 10500 | 84 |
| | 100 | | 0.5), 5-6 | beads | | | 250 | | |
| 7F | 50 to | 135 | (pH 6.5 ± | 100 μl | 1.25 | 12500 | 125- | 11200 | 90 |
| | 100 | | 0.5), 6-7 | beads | | | 250 | | |
| 8 | 150 to | 565 | (pH 4.5 ± | 100 μl | 1.25 | 12500 | 62.5- | 10400 | 83 |
| | 200 | | 0.5), 4-5 | beads | | | 83.3 | | |
| 9V | 50 to | 108 | (pH 5.5 ± | 100 μl | 1.25 | 12500 | 125- | 10100 | 81 |
| | 100 | | 0.5), 5-6 | beads | | | 250 | | |
| 10A | 50 to | 119 | (pH 6.5 ± | 100 μl | 1.25 | 12500 | 125- | 11300 | 90 |
| | 100 | | 0.5), 6-7 | beads | | | 250 | | |
| 11A | 200 to | 100 | (pH 5.5 ± | 100 μl | 1.25 | 12500 | 50- | 9960 | 80 |
| | 250 | | 0.5), 5-6 | beads | | | 62.5 | | |
| 12F | 50 to | 99 | (pH 6.5 ± | 100 μl | 1.25 | 12500 | 125- | 10500 | 84 |
| | 100 | | 0.5), 6-7 | beads | | | 250 | | |
| 14 | 200 to | 143 | (pH 6.5 ± | 100 μl | 1.25 | 12500 | 50- | 10800 | 86 |
| | 250 | | 0.5), 6-7 | beads | | | 62.5 | | |
| 15B | 200 to | 136 | (pH 5.5 ± | 100 μl | 1.25 | 12500 | 50- | 10400 | 83 |
| | 250 | | 0.5), 5-6 | beads | | | 62.5 | | |
| 18C | 200 to | 79 | (pH 6.5 ± | 100 μl | 1.25 | 12500 | 50- | 10700 | 86 |
| | 250 | | 0.5), 6-7 | beads | | | 62.5 | | |
| 19A | 50 to | 91 | (pH 5.5 ± | 100 μl | 1.25 | 12500 | 125- | 10000 | 80 |
| | 100 | | 0.5), 5-6 | beads | | | 250 | | |
| 19F | 50 to | 85 | (pH 4.5 ± | 100 μl | 1.25 | 12500 | 125- | 11400 | 91 |
| | 100 | | 0.5), 4-5 | beads | | | 250 | | |
| 22F | 200 to | 106 | (pH 5.5 ± | 100 μl | 1.25 | 12500 | 50- | 10300 | 82 |
| | 250 | | 0.5), 5-6 | beads | | | 62.5 | | |
| 23F | 200 to | 94 | (pH 4.5 ± | 100 μl | 1.25 | 12500 | 50- | 11500 | 92 |
| | 250 | | 0.5), 4-5 | beads | | | 62.5 | | |
| 24F | 50 to | 81 | (pH 6.5 ± | 100 μl | 1.25 | 12500 | 125- | 10100 | 81 |
| | 100 | | 0.5), 6-7 | beads | | | 250 | | |
| 33F | 200 to | 191 | (pH 6.5 ± | 100 μl | 1.25 | 12500 | 50- | 10700 | 86 |
| | 250 | | 0.5), 6-7 | beads | | | 62.5 | | |
| 3 | 200 to | 191 | (pH 6.5 ± | 100 μl | 1.25 | 12500 | 50- | 11200 | 90 |
| | 250 | | 0.5), 6-7 | beads | | | 62.5 | | |
| Hib | 1 to | 0.3 KD | (pH 5.5 ± | 100 μl | 1.25 | 12500 | 8333- | 10500 | 84 |
| | 1.5 | | 0.5), 5-6 | beads | | | 12500 | | |

Conc. = Conc. Of PS require for coupling for 100 μl, BN = No. of Beads in millions, BL = No.
of Beads per 100 μl.

The calculation uses Bead Stock solution of 100 μl beads for the Desired Bead conditions.

### Example 14: Neisseria Meningitis Saccharides

The specific conditions applied to Neisseria meningitis saccharides during dilution of the saccharide stock and further conjugation to the beads/microspheres gave the optimal MFI's to positive samples.

TABLE 17
NEISSERIA MENINGITIS SACCHARIDES provides for conditions
of pH and Concentration of Polysaccharides, molecular
size, incubation temperature and time.

| | | | | Mol | |
|---|---|---|---|---|---|
| | | | Saccharide | size | Time, |
| Serotype | Condition | pH (PBST) | Conc. (mg/mL) | (kDa) | Temperature |
| Men A | A | (pH 4.5 ± 0.5), 4-5 | 0.5 to 1.0 | 136 | 1 Hr/25° C. |
| Men A | B | (pH 5.5 ± 0.5), 5-6 | 1.0 to 1.5 | 136 | 1 Hr/25° C. |
| Men A | C | (pH 5.5 ± 0.5), 5-6 | 1.5 to 2.0 | 136 | 1 Hr/25° C. |
| Men A | D | (pH 6.5 ± 0.5), 6-7 | 0.5 to 1.0 | 136 | 1.5 Hr/25° C. |
| Men C | A | (pH 4.5 ± 0.5), 4-5 | 0.5 to 1.0 | 116 | 1 Hr/25° C. |
| Men C | B | (pH 5.5 ± 0.5), 5-6 | 0.5 to 1.0 | 116 | 1 Hr/37° C. |
| Men C | C | (pH 4.5 ± 0.5), 4-5 | 1.0 to 1.5 | 116 | 1.5 Hr/25° C. |
| Men C | D | (pH 5.5 ± 0.5), 5-6 | 0.5 to 1.0 | 116 | 1.5 Hr/25° C. |
| Men W | A | (pH 4.5 ± 0.5), 4-5 | 0.5 to 1.0 | 123 | 1 Hr/37° C. |
| Men W | B | (pH 5.5 ± 0.5), 5-6 | 1.0 to 1.5 | 123 | 2 Hr/25° C. |
| Men W | C | (pH 6.5 ± 0.5), 6-7 | 1.5 to 2.0 | 123 | 2 Hr/25° C. |
| Men W | D | (pH 6.5 ± 0.5), 6-7 | 0.5 to 1.0 | 123 | 1.5 Hr/25° C. |
| Men X | A | (pH 4.5 ± 0.5), 4-5 | 0.5 to 1.0 | 116 | 1 Hr/25° C. |
| Men X | B | (pH 5.5 ± 0.5), 5-6 | 0.5 to 1.0 | 116 | 1 Hr/25° C. |
| Men X | C | (pH 4.5 ± 0.5), 4-5 | 0.5 to 1.0 | 116 | 1 Hr/25° C. |
| Men X | D | (pH 4.5 ± 0.5), 4-5 | 0.5 to 1.0 | 116 | 1.5 Hr/37° C. |
| Men Y | A | (pH 4.5 ± 0.5), 4-5 | 0.5 to 1.0 | 106 | 1 Hr/25° C. |
| Men Y | B | (pH 5.5 ± 0.5), 5-6 | 0.5 to 1.0 | 106 | 1 Hr/25° C. |
| Men Y | C | (pH 6.5 ± 0.5), 6-7 | 0.5 to 1.0 | 106 | 1 Hr/25° C. |
| Men Y | D | (pH 6.5 ± 0.5), 6-7 | 0.5 to 1.0 | 106 | 1.5 Hr/37° C. |

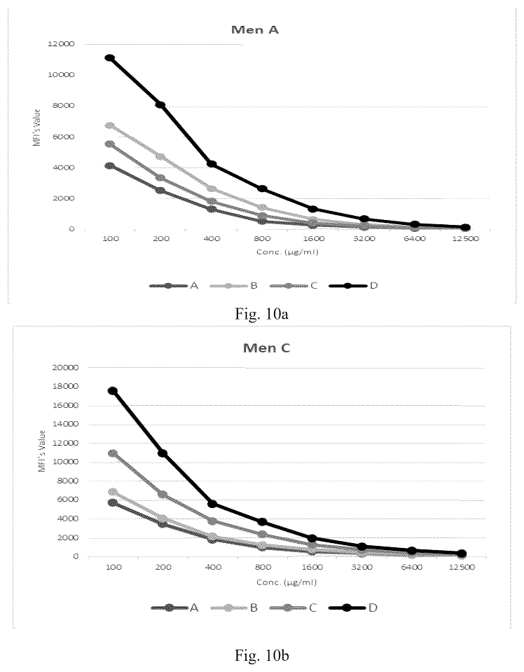

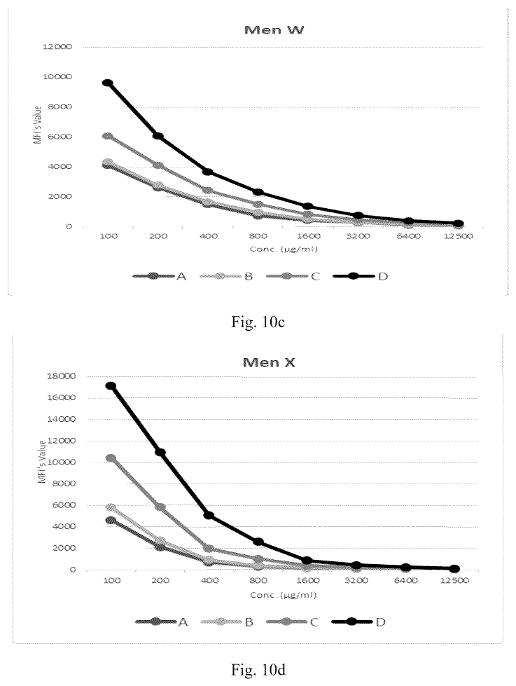

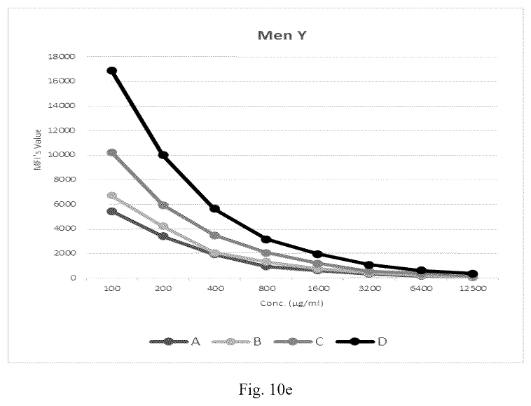

See FIG. 10 (10a to 10e) for details of MFI value against Neisseria meningitis saccharides of serotypes (A, C, W, X, Y respectively) in conditions of pH and Concentration of Polysaccharides, molecular size, incubation temperature and time.

### Key Features

The method provides non-covalent coupling of polysaccharides to microspheres to form couples under specific reaction conditions where the structure of polysaccharides remains unaffected.
The method includes specific reaction condition (Ps antigen specific) which allows efficient coupling of Ps on to the polystyrene beads.
The method includes specific combination of concentration of PnPS, size of Ps, reaction time, pH of buffer to allow development of electron donor sites which allow electrostatic attachment of Ps on to activated beads.
Method is associated with efficient coupling as an outcome of correct saccharide size, concentration and pH.
Efficiently coupled beads have following characteristics:
   a) Higher sensitivity
   b) Less noise/background
   c) Good precision (CV among duplicates) and accuracy (back fitted recoveries) d) Stable saccharide microsphere coupling
Method is aqueous based and highly stable and does not require fresh use of toxic chemicals
Method is fast and scalable to higher volumes
Method allows stable saccharide coupled microsphere/coupled beads that are robust and carries native antigens with no loss of activity
Saccharide coupled microsphere formed allows higher sensitivity of detection leading to development of assays with higher sensitivity.
Saccharide coupled microspheres showed good dilution linearity and no cross reactions
The sensitivity of the assay for the serotypes is increased with the saccharide coupled microsphere that subsequently lead to improved efficiency of the assays for vaccine potency determination.
Obtained saccharide coupled beads are stable at different temperatures.
The saccharide coupled microspheres is prepared using an activation buffer to activate the metal
The method provides following advantages
   Allows formation of stable saccharide coupled beads which are robust and carries saccharide with no loss of activity
   Highly stable aqueous based reaction.

### Advantages

Method is simple and easy to perform is replicable with different Saccharides and buffers.
Method is cost effective and do not need toxic compounds.

### APPLICATIONS

Method of forming the saccharide coupled microsphere is applicable for both magnetic and non-magnetic microspheres.
Method of forming the saccharide coupled microsphere is applicable for Saccharides of different microorganisms, bacteria, especially for streptococcal, meningococcal, Haemophilus, Salmonella saccharides.
Method of forming the saccharide coupled microsphere facilitates forming couples that are usable in serological testing of human and animals' sera samples, for testing of vaccines, for developing serological testing of vaccines in human sera samples
The method is applicable to develop immunoassays for Pneumococcal Purified Ps (PCV10, PCV21, PCV22), as well as other conjugate vaccines (Hib, Meningococcal, Typhi/Paratyphi and the like)
Saccharide coupled microspheres can be used for
   Antigen content estimation of PCV 10 (Pneumosil), PCV 21, PCV22.
   Serological testing of PCV 10, PCV 21, and PCV22 for use with animal sera and human sera samples
   Identity testing of PCV 10, PCV-21 PCV22, Hib, etc. samples.

What is claimed is:

1. A method of coupling a saccharide to a microsphere to obtain a saccharide coupled microsphere, the method comprising:
a. providing the microsphere;
b. providing the saccharide;
c. diluting the saccharide with a buffer at pH in range of 3.0 to 9.0; and
d. mixing the microsphere with the saccharide to form the saccharide coupled microsphere;
wherein the mixing further includes incubation at temperature in range of 20° C. to 40° C. for incubation time in range of 30 mins to 180 mins.

2. The method as claimed in claim 1, wherein
the mixing is performed with the coupling ratio of the microsphere to the saccharide is in range of 50 to 12500,
the microsphere is activated with a bead reagent before mixing with the saccharide, wherein the bead reagent includes metal ions, and
optionally, the saccharide coupled microsphere is formed by non-covalent electrostatic coupling.

3. The method as claimed in claim 1, wherein the buffer includes phosphate buffered saline with tween (PBST) buffer, (4-(2-hydroxyethyl)-piperazineethane sulfonic acid (HEPES) buffer, tris-aminomethane (Tris) buffer, 2-(N-morpholino)ethane sulfonic acid (MES) buffer, and 3-(N-morpholino) propane sulfonic acid (MOPS) buffer.

4. The method as claimed in claim 1, wherein the saccharide is bacterial saccharide including Group A Streptococcus, Group B Streptococcus, Streptococcus pneumoniae/pneumococcus, Haemophilus bacteria, Haemophilus influenzae bacteria, Haemophilus influenzae type b bacteria (Hib), Salmonella, Typhoidal salmonella, Non-typhoidal salmonella, Salmonella typhi, Salmonella typhimurium, Salmonella paratyphi, Streptococcus pyogenes, Streptococcus agalactiae, Shigella, Shigella dysenteriae, Shigella flexneri, Shigella sonnei, Escherichia coli, or Neisseria meningitidis/meningococcus saccharide.

5. The method as claimed in claim 4, wherein the saccharide is Streptococcus pneumoniae saccharide,
wherein the Streptococcus pneumoniae saccharide is selected from serotypes 1, 2, 3, 4, 5, 6, 6A, 6B, 6C, 6D, 7, 7B, 7C, 7F, 8, 9, 9A, 9L, 9N, 9V, 10, 10A, 10B, 10C, 1F, 10X, 11, 11A, 11B, 11C, 11F, 12, 12A, 12B, 12F, 13, 14, 15, 15A, 15B, 15BC, 15C, 15F, 16, 16F, 17, 17A, 17F, 18, 18A, 18B, 18C, 18F, 19, 19A, 19B, 19F, 20, 20A, 20B, 20F, 21, 22, 22A, 22F, 23, 23A, 23B, 23F, 24, 24F, 25, 25F, 26, 27, 28, 28A, 28F, 29, 30, 31, 32, 33, 33A, 33B, 33C, 33D, 33F, 34, 35, 35A, 35B, 35C, 35D, 35F, 36, 37, 38, 39, 40, 41, 42, 43, 44, 45, 46, 47, and/or 48, and
wherein the Streptococcus pneumoniae saccharide has a molecular size in range of 50 kDa to 3000 kDa, is diluted with PBST buffer at a pH in range of 3.0 to 9.0 and has concentration of 0.05 mg/mL to 50.0 mg/mL.

6. The method as claimed in claim 5, wherein the Streptococcus pneumoniae saccharide is mixed with the microsphere and incubated at temperature of 23° C. to 39° C., for incubation time of 60 mins to 120 mins.

7. The method as claimed in claim 4, wherein the saccharide is Haemophilus influenzae type b bacteria (Hib) saccharide, and
wherein the Haemophilus influenzae type b bacteria (Hib) saccharide has a molecular size in range of 0.05 kDa to 5 kDa, is diluted with PBST buffer at a pH in range of 4 to 6 and has concentration of 1.0 μg/mL to 50.0 μg/mL.

8. The method as claimed in claim 7, wherein the Haemophilus influenzae type b bacteria (Hib) saccharide is mixed with the microsphere and incubated at temperature of 20° C. to 30° C., for incubation time of 60 mins to 120 mins.

9. The method as claimed in claim 4, wherein the saccharide is Neisseria meningitidis saccharide serotypes selected from meningococcal serotypes A (type I and III), B (type II), B6, B16, C (type II-alpha), D (type IV), Z′/E, E29, H, I, K, K454, L, M, W135, X, Y, Z, and
wherein the Neisseria meningitidis saccharide has a molecular size in range of 75 kDa to 3000 kDa, is diluted with PBST buffer at a pH in range of 4 to 7 and has concentration of 0.01 mg/mL to 10.0 mg/mL.

10. The method as claimed in claim 9, wherein the Neisseria meningitidis saccharide is mixed with the microsphere and incubated at temperature of 20 to 40° C., for incubation time of 60 mins to 120 mins.

11. A saccharide coupled microsphere obtained by the method as claimed in claim 1.

12. The saccharide coupled microsphere as claimed in claim 11, wherein the saccharide coupled microsphere has Mean Florescence intensity/MFI value in the range of 200 to 20000.

13. The saccharide coupled microsphere as claimed in claim 11, wherein the saccharide coupled microsphere is a Streptococcus pneumoniae saccharide coupled microsphere.

14. The saccharide coupled microsphere as claimed in claim 11, wherein the saccharide coupled microsphere is a Haemophilus influenzae type b bacteria (Hib) saccharide coupled microsphere.

15. The saccharide coupled microsphere as claimed in claim 11, wherein the saccharide coupled microsphere is a Neisseria meningitidis saccharide coupled microsphere.

16. A method of evaluating immunogenicity of immunogenic composition, the method comprising;
a. providing a test sample corresponding to the saccharide in the immunogenic composition;
b. providing the saccharide coupled microsphere corresponding to the saccharide in the immunogenic composition; and
c. evaluating the immunogenicity of the immunogenic composition by antigen content determination, or estimating antibody concentration (IgG), or identity assay, free polysaccharide estimation using the test sample and the saccharide coupled microsphere,
wherein the saccharide coupled microsphere is obtained by the method as claimed in claim 1.
